SEQUENCE LISTING (11 sequences)

SEQ ID NO: 1 - protein, 321 aa, Homo sapiens
MAPAFLLLLLLWPQGCVSGPSADSVYTKVRLLEGETLSVQCSYKGYKNRVEGKVWCKIRKKKCEPGFARVWVKGPRYLLQDDAQAKVVNITMVALKLQDSGRYWCMRNTSGILYPLMGFQLDVSPAPQTERNIPFTHLDNILKSGTVTTGQAPTSGPDAPFTTGVMVFTPGLITLPRLLASTRPASKTGYSFTATSTTSQGPRRTMGSQTVTASPSNARDSSAGPESISTKSGDLSTRSPTTGLCLTSRSLLNRLPSMPSIRHQDVYSTVLGVVLTLLVLMLIMVYGFWKKRHMASYSMCSDPSTRDPPGRPEPYVEVYLI

SEQ ID NO: 2 - protein, 250 aa, Homo sapiens
GPSADSVYTKVRLLEGETLSVQCSYKGYKNRVEGKVWCKIRKKKCEPGFARVWVKGPRYLLQDDAQAKVVNITMVALKLQDSGRYWCMRNTSGILYPLMGFQLDVSPAPQTERNIPFTHLDNILKSGTVTTGQAPTSGPDAPFTTGVMVFTPGLITLPRLLASTRPASKTGYSFTATSTTSQGPRRTMGSQTVTASPSNARDSSAGPESISTKSGDLSTRSPTTGLCLTSRSLLNRLPSMPSIRHQDVYS

SEQ ID NO: 3 - protein, 63 aa, Homo sapiens
SADSVYTKVRLLEGETLSVQCSYKGYKNRVEGKVWCKIRKKKCEPGFARVWVKGPRYLLQDDA

SEQ ID NO: 4 - protein, 50 aa, Homo sapiens
GRYWCMRNTSGILYPLMGFQLDVSPAPQTERNIPFTHLDNILKSGTVTTG

SEQ ID NO: 5 - protein, 50 aa, Homo sapiens
TGYSFTATSTTSQGPRRTMGSQTVTASPSNARDSSAGPESISTKSGDLST

SEQ ID NO: 6 - protein, 10 aa, Mus musculus
GFSLSTSGMG

SEQ ID NO: 7 - protein, 7 aa, Mus musculus
IWWYDDK

SEQ ID NO: 8 - protein, 12 aa, Mus musculus
VRIESTMITGDY

SEQ ID NO: 9 - protein, 10 aa, Mus musculus
QSVDYDGYSY

SEQ ID NO: 10 - protein, 3 aa, Mus musculus
AAS

SEQ ID NO: 11 - protein, 9 aa, Mus musculus
QQSIEDPWT

(19) United States
(12) Patent Application Publication
(10) Pub. No.: US 2022/0235420 A1
(43) Pub. Date: Jul. 28, 2022

(54) COMPOSITIONS AND METHODS FOR ISOLATING, DETECTING, AND ANALYZING FETAL CELLS

(71) Applicant: MENARINI BIOMARKERS SINGAPORE PTE LTD, Singapore (SG)

(72) Inventors: Paola Castagnoli, Greve in Chianti (IT); Anna Doffini, Singapore (SG); Wen-Shan Tsao, Taipei City (TW)

(73) Assignee: MENARINI BIOMARKERS SINGAPORE PTE LTD, Singapore (SG)

(21) Appl. No.: 17/627,476

(22) PCT Filed: Jul. 14, 2020
(86) PCT No.: PCT/IB2020/056632

§ 371 Date: Jan. 14, 2022

Related U.S. Application Data
(60) Provisional application No. 62/874,306, filed on Jul. 15, 2019.

(51) Int. Cl. C12Q 1/6883; G01N 33/543; G01N 33/569; C07K 16/28

(52) U.S. Cl. CPC C12Q 1/6883; G01N 33/54326; G01N 33/56966; C12Q 1/6869; G01N 2333/705; C12Q 2600/156; C07K 16/2803

(57) ABSTRACT

Compositions, kits, and methods for isolating, detecting, and analyzing fetal cells are provided. Methods for preparing a fetal cell sample and for performing fetal genetic testing are also provided herein. The compositions, kits, and methods may comprise use an anti-TREML2 antibody. Alternatively, or additionally, the compositions, kits, and methods comprise or use an antibody conjugated to a colloidal magnetic particle and/or an exogenous aggregation enhancing factor.

### CROSS-REFERENCE TO RELATED APPLICATIONS

This application claims priority to U.S. Provisional Application No. 62/874,306, filed Jul. 15, 2019, the disclosure of which is incorporated by reference in its entirety.

### BACKGROUND

In the last four decades investigators have tried to isolate fetal cells in pregnant women to develop prenatal diagnostic tools. In the early 1970s, amniocentesis was first developed followed by chorionic villus sampling (CVS) developed in the 1980s. Amniocentesis and chorionic villus sampling (CVS) are the two invasive methods used in routine clinical practice for the diagnosis of chromosomal abnormalities such as common fetal aneuploidy (an extra copy of a chromosome) e.g. trisomy of chromosomes 13,18 and 21 (leading to Down syndrome).

The ability to isolate fetal cells and fetal DNA from maternal blood during pregnancy has opened up exciting opportunities for improved noninvasive prenatal testing. Recently, a cell-free DNA-based screening (cfDNA) known as non-invasive prenatal testing (NIPT) was introduced in prenatal screening and has been recognized as highly predictive for trisomy 21. Nevertheless the screening performances are below that of the invasive diagnostic tools and confirmatory tests are still necessary. Furthermore, NIPT is not predictive of copy number variants (CNVs) or microdeletions/duplications, according to professional societies (Practice bulletin n163 Obstet Gynecol. 2016; 127(5) 979-981). Thus, current cell-free NIPT is not yet adequate for detecting subchromosomal deletions and duplications with high specificity, sensitivity, and positive predictive value.

Direct analysis of fetal cells from maternal circulation has been challenging so far given the scarcity of fetal cells in maternal blood. Many different methods of enrichment have been tested including filters, density gradients, fluorescence activated cell sorting (FACS), microfluidics, and immuno-magnetic beads. Although circulating fetal cells can be recovered, these methods have lacked consistency and reproducibility. This is due to the extremely low number of circulating fetal cells (0.1-10 cells in 1 ml of maternal blood which contains about 1-5 million cells) that has hampered so far the establishment of reproducible protocols. The challenge is to eliminate all the contaminating nucleated blood cells without losing the very few circulating fetal cells in the first trimester of gestation.

Taking into account these limitations, and the fact that amniocentesis and chorionic villus sampling (CVS) are procedures with related risk for pregnancy loss, there is a need to develop new cell-based NIPD (non-invasive prenatal diagnosis) procedures to select fetal cells from maternal blood of pregnant women in order to screen for birth defects and inherited diseases.

Fetal nucleated red blood cells (nRBC) and trophoblastic cells are known to be present in the maternal circulation, but it has been difficult to develop a reliable cytogenetic cell-based form of NIPT. Recently the possibility of developing a cell-based form of NIPT with ability to detect abnormalities with a similar accuracy as can currently be obtained with amniocentesis and CVS has been proposed (Amy M. Breman, et al., Prenatal Diagnosis, 2016, 36(11):1009-1019).

Disclosed herein are fetal cell markers and agents that bind them. Further disclosed herein are compositions, kits, and methods for isolating, detecting, and analyzing fetal cells based on fetal cell markers.

### SUMMARY OF THE INVENTION

Disclosed herein is a method for detecting fetal cells in a sample from a pregnant subject, comprising: (a) contacting the sample with a first antibody, wherein the sample comprises a plurality of cells; (b) isolating cells bound to the first antibody to produce an enriched sample; (c) contacting the enriched sample with a second antibody; and (d) identifying a cell that is bound to the second antibody as a fetal cell, wherein the first antibody or the second antibody: (i) is an antibody that binds to a Triggering Receptor Expressed on Myeloid Cells Like 2 (TREML2) protein; or (ii) comprises an antigen binding fragment that binds to a TREML2 protein.

In some embodiments, the fetal cell is a fetal nucleated red blood cell (fnRBC). In some embodiments, the fetal cell is a trophoblast.

In some embodiments, the first antibody is conjugated to one or more magnetic particles. In some embodiments, the magnetic particles are colloidal magnetic particles. In some embodiments, the colloidal magnetic particles are ferrofluid magnetic particles. In some embodiments, the magnetic particles are coupled to a first exogenous aggregation enhancing factor (EAEF), the first EAEF comprising one member of a specific binding pair selected from the group comprising biotin-streptavidin, antigen-antibody, receptor-hormone, receptor-ligand, agonist-antagonist, lectin-carbohydrate, Protein A-antibody Fc, and avidin-biotin, biotin analog-avidin, desthiobiotin-streptavidin, desthiobiotin-avidin, iminobiotin-streptavidin, and iminobiotin-avidin.

In some embodiments, step (a) comprises adding a second EAEF to induce aggregation of the magnetic particles, the second EAEF comprising the other member of the specific binding pair.

In some embodiments, step (b) comprises subjecting the sample to a magnetic field.

In some embodiments, step (b) comprises adding to the enriched sample a member of the specific binding pair in order to reverse aggregation of the magnetic particles in the enriched sample.

In some embodiments, the method further comprises, prior to step (a), adding to the sample at least one aggregation inhibiting agent selected from the group consisting of a reducing agent, an immune-complex, a chelating agent, and a diamino butane. In some embodiments, the aggregation inhibiting agent is a chelating agent. In some embodiments, the chelating agent is ethylenediaminetetraacetic acid (EDTA).

In some embodiments, the second antibody is an antibody that binds to TREML2 protein or comprises an antigen binding fragment that binds to a TREML2 protein. In some embodiments, the TREML2 protein comprises, consists of, or consists essentially of the amino acid sequence as set forth in SEQ ID NO: 1. In some embodiments, the TREML2 protein comprises, consists of, or consists essentially of the amino acid sequence as set forth in any one of SEQ ID Nos: 2-5.

In some embodiments, the method further comprises, prior to step (d), isolating single fetal cells.

In some embodiments, isolating single fetal cells is carried out by isolating single fetal cells that are bound to the second antibody.

In some embodiments, the second antibody which is conjugated to a label. In some embodiments, the label is a fluorescent label. In some embodiments, the label is selected from phycoerythrin (PE), allophycocyanin (APC), horse radish peroxidase (HRP), and biotin.

In some embodiments, isolating single fetal cells is based on immunofluorescent technology. In some embodiments, isolating single fetal cells is carried out by fluorescence activated cell sorting (FACS). In some embodiments, isolating single cells is carried out with a DEPArray.

In some embodiments, step (d) comprises performing a sequencing analysis. In some embodiments, the sequencing analysis comprises short tandem repeat (STR) analysis.

In some embodiments, the method further comprises analyzing the fetal cell. In some embodiments, analyzing the fetal cell comprises performing a genomic or a genetic analysis. In some embodiments, performing a genetic analysis comprises detecting the presence or absence of one or more genetic abnormalities in the fetal cell.

In some embodiments, the first antibody is an antibody that binds to a TREML2 protein or comprises an antigen binding fragment that binds to a TREML2 protein. In some embodiments, the TREML2 protein comprises, consists of, or consists essentially of the amino acid sequence as set forth in SEQ ID NO: 1. In some embodiments, the TREML2 protein comprises, consists of, or consists essentially of the amino acid sequence as set forth in any one of SEQ ID Nos: 2-5.

In some embodiments, the antibody that binds to a TREML2 protein or an antigen binding fragment that binds to a TREML2 protein comprises one or more CDRs selected from: (i) a heavy chain variable region (HCVR) complementarity determining region (CDR) 1 comprising the amino acid sequence of SEQ ID NO: 6; (ii) a HCVR CDR2 comprising the amino acid sequence of SEQ ID NO: 7; (iii) a HCVR CDR3 comprising the amino acid sequence of SEQ ID NO: 8; (iv) a light chain variable region (LCVR) CDR1 comprising the amino acid sequence of SEQ ID NO: 9; (v) a LCVR CDR2 comprising the amino acid sequence of SEQ ID NO: 10; and (vi) a LCVR CDR3 comprising the amino acid sequence of SEQ ID NO: 11. In some embodiments, any one of SEQ ID Nos: 6-11 independently comprise one or more amino acid substitutions, additions, or deletions. In some embodiments, the antibody that binds to a TREML2 protein or an antigen binding fragment that binds to a TREML2 protein comprises 2, 3, 4, 5 or 6 of the CDRs selected from (i)-(vi).

In some embodiments, the antibody that binds to a TREML2 protein is an anti-TREML2 antibody. In some embodiments, the anti-TREML2 antibody is selected from sc-109096, ARP49877_P050, OACA04996, AF3259, MA5-30973, PAS-47471, ABIN634968, ABIN928294, 30-552, ABIN2463297, ABIN19999041, 11655-r001, ABIN749888, bs-2737r, ABIN1999045, 11655-rp02, ABIN293207, ABIN2387613, t8282-40, ABIN4249314, nbp1-70737-20ul, and BD563661.

Further disclosed herein is a method for detecting fetal cells in a sample from a pregnant subject, comprising: (a) contacting the sample with a magnetic reagent, wherein the sample comprises a plurality of cells, wherein the magnetic reagent comprises a magnetic particle conjugated to a first antibody, and wherein the first antibody binds to a protein selected from EpCAM, CD105, and CD71; (b) contacting the sample with an anti-TREML2 antibody or antigen binding fragment thereof; and (c) identifying a cell that is bound to the anti-TREML2 antibody as a fetal cell.

In some embodiments, the method further comprises, prior to step (c), isolating cells bound to the first antibody. In some embodiments, isolating cells comprises subjecting the sample to a magnetic field in order to enrich the sample with cells that are bound to the first antibody.

In some embodiments, the magnetic particle is a colloidal magnetic particle. In some embodiments, the colloidal magnetic particle is a ferrofluid magnetic particle. In some embodiments, the colloidal magnetic particle is less than 200 nm. In some embodiments, the colloidal magnetic particle is between about 80 to 200 nm. In some embodiments, the colloidal magnetic particle is between about 90 to 150 nm. In some embodiments, the colloidal magnetic particle has a magnetic mass of at least 50%. In some embodiments, the colloidal magnetic particle has a magnetic mass of at least 60%. In some embodiments, the colloidal magnetic particle has a magnetic mass of between 70% to 90%. In some embodiments, the colloidal magnetic particle comprises a crystalline core of a superparamagnetic material that is surrounded by coating molecules.

In some embodiments, the magnetic particle is further coupled to a first exogenous aggregation enhancing factor (EAEF), the first EAEF comprises one member of a specific binding pair selected from the group comprising biotin-streptavidin, antigen-antibody, receptor-hormone, receptor-ligand, agonist-antagonist, lectin-carbohydrate, Protein A-antibody Fc, and avidin-biotin, biotin analog-avidin, desthiobiotin-streptavidin, desthiobiotin-avidin, iminobiotin-streptavidin, and iminobiotin-avidin.

In some embodiments, the method comprises adding, during step (a), a second EAEF to increase aggregation of the particles wherein the second EAEF comprising the other member of the specific binding pair.

In some embodiments, the method further comprises adding to the enriched sample a member of the specific binding pair (third EAEF) in order to reverse aggregation of the magnetic reagent in the sample, thereby facilitating identification of the cell.

In some embodiments, the method further comprises, prior to step (a), adding to the sample at least one aggregation inhibiting agent selected from the group consisting of a reducing agent, an immune-complex, a chelating agent, a diamino butane. In some embodiments, the aggregation inhibiting agent is a chelating agent. In some embodiments, the chelating agent is EDTA.

In some embodiments, the method further comprises, prior to step (c), isolating the cell using the anti-TREML2 antibody or a second antibody. In some embodiments, the second antibody is selected from an anti-cytokeratin antibody and anti-HLAG antibody.

In some embodiments, the anti-TREML2 antibody or the second antibody is conjugated to a label. In some embodiments, the label is a fluorescent label. In some embodiments, the label is selected from phycoerythrin (PE), allophycocyanin (APC), horse radish peroxidase (HRP), and biotin.

In some embodiments, isolating the cell is based on immunofluorescent technology. In some embodiments, isolating the cell is carried out by fluorescence activated cell sorting (FACS). In some embodiments, isolating the cell is carried out with a DEPArray.

In some embodiments, identifying the cell comprises performing a sequencing analysis.

In some embodiments, the sequencing analysis comprises short tandem repeat (STR) analysis.

In some embodiments, the method further comprises analyzing the fetal cell. In some embodiments, analyzing the fetal cell comprises performing a genomic or a genetic analysis. In some embodiments, performing a genetic analysis comprises detecting the presence or absence of one or more genetic abnormalities in the fetal cell.

In some embodiments, the fetal cell is a fetal erythroblast or fetal trophoblast.

In some embodiments, the anti-TREML2 antibody or antigen binding fragment thereof comprises one or more complementarity determining regions (CDRs) selected from: (i) a heavy chain variable region (HCVR) CDR1 comprising the amino acid sequence of SEQ ID NO: 6; (ii) a HCVR CDR2 comprising the amino acid sequence of SEQ ID NO: 7; (iii) a HCVR CDR3 comprising the amino acid sequence of SEQ ID NO: 8; (iv) a light chain variable region (LCVR) CDR1 comprising the amino acid sequence of SEQ ID NO: 9; (v) a LCVR CDR2 comprising the amino acid sequence of SEQ ID NO: 10; and (vi) a LCVR CDR3 comprising the amino acid sequence of SEQ ID NO: 11. In some embodiments, any one of SEQ ID Nos: 6-11 independently comprise one or more amino acid substitutions, additions, or deletions.

In some embodiments, the anti-TREML2 antibody is selected from sc-109096, ARP49877_P050, OACA04996, AF3259, MA5-30973, PAS-47471, ABIN634968, ABIN928294, 30-552, ABIN2463297, ABIN19999041, 11655-r001, ABIN749888, bs-2737r, ABIN1999045, 11655-rp02, ABIN293207, ABIN2387613, t8282-40, ABIN4249314, nbp1-70737-20ul, and BD563661.

Further disclosed herein is a method for detecting fetal cells in a sample from a pregnant subject, comprising: (a) contacting the sample with a first antibody, wherein the sample comprises a plurality of cells, and wherein the first antibody binds to a Triggering Receptor Expressed on Myeloid Cells Like 2 (TREML2) protein (anti-TREML2 antibody) or an antigen binding fragment thereof; and (b) identifying a cell that is bound to the first antibody as a fetal cell.

In some embodiments, the fetal cell is a fetal nucleated red blood cell (fnRBC).

In some embodiments, the first antibody is conjugated to one or more magnetic particles. In some embodiments, the magnetic particles are colloidal magnetic particles. In some embodiments, the colloidal magnetic particles are ferrofluid magnetic particles. In some embodiments, the colloidal magnetic particle is less than 200 nm. In some embodiments, the colloidal magnetic particle is between about 80 to 200 nm. In some embodiments, the colloidal magnetic particle is between about 90 to 150 nm. In some embodiments, the colloidal magnetic particle has a magnetic mass of at least 50%. In some embodiments, the colloidal magnetic particle has a magnetic mass of at least 60%. In some embodiments, the colloidal magnetic particle has a magnetic mass of between 70% to 90%. In some embodiments, the colloidal magnetic particle comprises a crystalline core of a superparamagnetic material that is surrounded by coating molecules.

In some embodiments, the method further comprises subjecting the sample to a magnetic field.

In some embodiments, the magnetic particles are coupled to a first exogenous aggregation enhancing factor (EAEF), wherein the first EAEF comprises one member of a specific binding pair selected from the group comprising biotin-streptavidin, antigen-antibody, receptor-hormone, receptor-ligand, agonist-antagonist, lectin-carbohydrate, Protein A-antibody Fc, and avidin-biotin, biotin analog-avidin, desthiobiotin-streptavidin, desthiobiotin-avidin, iminobiotin-streptavidin, and iminobiotin-avidin.

In some embodiments, the method further comprises, prior to step (b), adding a second EAEF to increase aggregation of the magnetic particles, wherein the second EAEF comprises the other member of the specific binding pair.

In some embodiments, the method further comprises isolating cells that are bound to the first antibody to produce an enriched sample.

In some embodiments, the method further comprises adding to the enriched sample a third EAEF in order to reverse aggregation of the magnetic particles in the enriched sample, wherein the third EAEF is capable of binding to the first EAEF or the second EAEF. In some embodiments, the third EAEF is a member of the specific binding pair.

In some embodiments, the method further comprises, prior to step (a), adding to the sample at least one aggregation inhibiting agent selected from the group consisting of a reducing agent, an immune-complex, a chelating agent, and a diamino butane. In some embodiments, the aggregation inhibiting agent is the chelating agent. In some embodiments, the chelating agent is EDTA.

In some embodiments, the first antibody is conjugated to a label. In some embodiments, the label is a fluorescent label. In some embodiments, the label is selected from phycoerythrin (PE), allophycocyanin (APC), horse radish peroxidase (HRP), and biotin.

In some embodiments, the method further comprises, prior to step (b), isolating cells that are bound to the first antibody, wherein isolating cells is based on immunofluorescent technology. In some embodiments, isolating cells that are bound to the first antibody is carried out by fluorescence activated cell sorting (FACS). In some embodiments, isolating cells that are bound to the first antibody is carried out with a DEPArray.

In some embodiments, step (b) comprises performing a sequencing analysis. In some embodiments, the sequencing analysis comprises short tandem repeat (STR) analysis. In some embodiments, the method further comprises analyzing the fetal cell. In some embodiments, analyzing the fetal cell comprises performing a genomic or a genetic analysis. In some embodiments, performing a genetic analysis comprises detecting the presence or absence of one or more genetic abnormalities in the fetal cell.

In some embodiments, the first antibody or antigen binding fragment thereof comprises, consists of, or consists essentially of, one or more CDRs selected from: (a) a HCVR CDR1 comprising the amino acid sequence of SEQ ID NO: 6; (b) a HCVR CDR2 comprising the amino acid sequence of SEQ ID NO: 7; (c) a HCVR CDR3 comprising the amino acid sequence of SEQ ID NO: 8; (d) a LCVR CDR1 comprising the amino acid sequence of SEQ ID NO: 9; (e) a LCVR CDR2 comprising the amino acid sequence of SEQ ID NO: 10; and (f) a LCVR CDR3 comprising the amino acid sequence of SEQ ID NO: 11. In some embodiments, any of SEQ ID Nos: 6-11 independently comprise one or more amino acid substitutions, additions, or deletions. In some embodiments, the first antibody, or an antigen binding fragment thereof, comprises, consists of, or consists essentially of, 2, 3, 4, 5 or 6 of the CDRs selected from (a)-(f).

In some embodiments, the first antibody is selected from sc-109096, ARP49877_P050, OACA04996, AF3259, MA5-30973, PAS-47471, ABIN634968, ABIN928294, 30-552, ABIN2463297, ABIN19999041, 11655-r001, ABIN749888, bs-2737r, ABIN1999045, 11655-rp02, ABIN293207, ABIN2387613, t8282-40, ABIN4249314, nbpl-70737-20ul, and BD563661.

Further disclosed herein is a method for cell-based fetal genetic testing, comprising: (a) contacting a sample obtained from a pregnant subject with an anti-TREML2 antibody or antigen binding fragment thereof, wherein the sample comprises a plurality of cells; (b) isolating cells that are bound to the anti-TREML2 antibody or antigen binding fragment thereof; (c) analyzing one or more nucleic acid molecules from the cells that are bound to the anti-TREML2 antibody or antigen binding fragment thereof; and (d) generating a report based on the analysis of the one or more nucleic acid molecules, wherein the report provides the likelihood of a fetus having one or more genetic abnormalities.

In some embodiments, the cells that are bound to the anti-TREML2 antibody or antigen binding fragment thereof are fetal cells.

In some embodiments, the fetal cells are fetal erythroblasts. In some embodiments, the fetal cells are fetal trophoblasts.

In some embodiments, analyzing the one or more nucleic acid molecules comprised conducting a karyotype analysis.

In some embodiments, analyzing the one or more nucleic acid molecules comprises performing a sequencing analysis. In some embodiments, the sequencing analysis comprises short tandem repeat (STR) analysis.

In some embodiments, the one or more genetic abnormalities is selected from a trisomy, sex chromosome anomaly, and structural anomaly. In some embodiments, the trisomy is selected from trisomy 3, trisomy 4, trisomy 6, trisomy 7, trisomy 8, trisomy 9, trisomy 10, trisomy 11, trisomy 12, trisomy 13, trisomy 16, trisomy 17, trisomy 18, trisomy 20, trisomy 21, and trisomy 22. In some embodiments, the sex chromosome anomaly is selected from monosomy X, triple X, and Klinefelter's syndrome. In some embodiments, the structural anomaly is a copy number variation (CNV). In some embodiments, the structural anomaly is a deletion of the CNV or duplication of the CNV.

In some embodiments, the anti-TREML2 antibody is conjugated to a magnetic particle. In some embodiments, the magnetic particle is a colloidal magnetic particle. In some embodiments, the colloidal magnetic particle is a ferrofluid magnetic particle.

In some embodiments, step (b) comprises subjecting the sample to a magnetic field.

In some embodiments, the method further comprises, prior to step (a), contacting the sample with a first antibody, wherein the first antibody binds to a protein selected from EpCAM, CD105, and CD71.

In some embodiments, further comprises, prior to step (a), isolating cells that are bound to the first antibody.

In some embodiments, the first antibody is conjugated to a magnetic particle. In some embodiments, the magnetic particle is a colloidal magnetic particle. In some embodiments, the colloidal magnetic particle is a ferrofluid magnetic particle. In some embodiments, the colloidal magnetic particle is less than 200 nm. In some embodiments, the colloidal magnetic particle is between about 80 to 200 nm. In some embodiments, the colloidal magnetic particle is between about 90 to 150 nm. In some embodiments, the colloidal magnetic particle has a magnetic mass of at least 50%. In some embodiments, the colloidal magnetic particle has a magnetic mass of at least 60%. In some embodiments, the colloidal magnetic particle has a magnetic mass of between 70% to 90%. In some embodiments, the colloidal magnetic particle comprises a crystalline core of a superparamagnetic material that is surrounded by coating molecules.

In some embodiments, isolating cells that are bound to the first antibody comprises subjecting the sample to a magnetic field. In some embodiments, the anti-TREML2 antibody or antigen binding fragment thereof is conjugated to a label. In some embodiments, the label is a fluorescent label.

In some embodiments, isolating cells that are bound to the anti-TREML2 antibody or antigen binding fragment thereof is based on immunofluorescent technology. In some embodiments, isolating cells that are bound to the anti-TREML2 antibody or antigen binding fragment thereof is carried out by fluorescence activated cell sorting (FACS). In some embodiments, isolating cells that are bound to the anti-TREML2 antibody or antigen binding fragment thereof is carried out with a DEPArray.

In some embodiments, the method further comprises contacting the cells that are bound to the anti-TREML2 antibody or antigen binding fragment thereof with a second antibody or antigen binding fragment thereof.

In some embodiments, the second antibody is an anti-TREML2 antibody or antigen binding fragment thereof. In some embodiments, the second antibody is conjugated to a label. In some embodiments, the label is a fluorescent label.

In some embodiments, the method further comprises isolating cells that are bound to the second antibody or antigen binding fragment thereof. In some embodiments, isolating cells that are bound to the second antibody or antigen binding fragment thereof is based on immunofluorescent technology. In some embodiments, isolating cells that are bound to the second antibody or antigen binding fragment thereof is carried out by fluorescence activated cell sorting (FACS). In some embodiments, isolating cells that are bound to the second antibody or antigen binding fragment thereof is carried out with a DEPArray.

In some embodiments, the anti-TREML2 antibody is selected from sc-109096, ARP49877_P050, OACA04996, AF3259, MA5-30973, PAS-47471, ABIN634968, ABIN928294, 30-552, ABIN2463297, ABIN19999041, 11655-r001, ABIN749888, bs-2737r, ABIN1999045, 11655-rp02, ABIN293207, ABIN2387613, t8282-40, ABIN4249314, nbp1-70737-20ul, and BD563661.

In some embodiments, the anti-TREML2 antibody, or an antigen binding fragment thereof, comprises, consists of, or consists essentially of, one or more CDRs selected from: (a) a HCVR CDR1 comprising the amino acid sequence of SEQ ID NO: 6; (b) a HCVR CDR2 comprising the amino acid sequence of SEQ ID NO: 7; (c) a HCVR CDR3 comprising the amino acid sequence of SEQ ID NO: 8; (d) a LCVR CDR1 comprising the amino acid sequence of SEQ ID NO: 9; (e) a LCVR CDR2 comprising the amino acid sequence of SEQ ID NO: 10; and (f) a LCVR CDR3 comprising the amino acid sequence of SEQ ID NO: 11. In some embodiments, any one of SEQ ID Nos: 6-11 independently comprise one or more amino acid substitutions, additions or deletions. In some embodiments, the anti-TREML2 antibody, or antigen binding fragment thereof, comprises, consists of, or consists essentially of, 2, 3, 4, 5 or 6 of the CDRs selected from (i)-(vi).

Further disclosed herein is a method of preparing a fetal cell sample from a maternal sample obtained from a pregnant subject, comprising: (a) contacting the maternal sample that comprises fetal cells and maternal cells with a first antibody conjugate, wherein the first antibody conjugate comprises (i) a first antibody; and (ii) a colloidal magnetic particle, wherein the first antibody is conjugated to the colloidal magnetic particle; and (b) isolating cells that are bound to the first antibody conjugate by subjecting the maternal sample to a magnetic field, thereby preparing a fetal cell sample.

In some embodiments, the magnetic particle is a colloidal magnetic particle. In some embodiments, the colloidal magnetic particle is a ferrofluid magnetic particle. In some embodiments, the colloidal magnetic particle is less than 200 nm. In some embodiments, the colloidal magnetic particle is between about 80 to 200 nm. In some embodiments, the colloidal magnetic particle is between about 90 to 150 nm. In some embodiments, the colloidal magnetic particle has a magnetic mass of at least 50%. In some embodiments, the colloidal magnetic particle has a magnetic mass of at least 60%. In some embodiments, the colloidal magnetic particle has a magnetic mass of between 70% to 90%. In some embodiments, the colloidal magnetic particle comprises a crystalline core of a superparamagnetic material that is surrounded by coating molecules.

In some embodiments, the magnetic particle is coupled to a first exogenous aggregation enhancing factor (EAEF), wherein the first EAEF comprises one member of a specific binding pair selected from the group comprising biotin-streptavidin, antigen-antibody, receptor-hormone, receptor-ligand, agonist-antagonist, lectin-carbohydrate, Protein A-antibody Fc, and avidin-biotin, biotin analog-avidin, desthiobiotin-streptavidin, desthiobiotin-avidin, iminobiotin-streptavidin, and iminobiotin-avidin.

In some embodiments, the method further comprises adding a second EAEF to the maternal sample, wherein the second EAEF comprises the other member of the specific binding pair.

In some embodiments, the first antibody is an anti-TREML2 antibody.

In some embodiments, the first antibody is an anti-CD71 antibody.

In some embodiments, the first antibody binds to a protein selected from EpCAM and CD105.

In some embodiments, preparing the fetal cell sample further comprises contacting the cells that are isolated from the maternal sample with a second antibody.

In some embodiments, the second antibody is conjugated to a label. In some embodiments, the label is a fluorescent label.

In some embodiments, preparing the fetal cell sample further comprises isolating cells that are bound to the second antibody.

In some embodiments, isolating cells that are bound to the second antibody is based on immunofluorescent technology. In some embodiments, isolating cells that are bound to the second antibody is carried out by fluorescence activated cell sorting (FACS). In some embodiments, isolating cells that are bound to the second antibody is carried out with a DEPArray.

Further disclosed herein is a method for detecting fetal cells in a sample from a pregnant subject, comprising: (a) contacting the sample with a first antibody conjugate, wherein the sample comprises a plurality of cells, and wherein the first antibody comprises a first antibody conjugated to a colloidal magnetic particle; (b) isolating cells bound to the first antibody by subjecting the sample to a magnetic field, thereby producing an enriched sample; (c) contacting the enriched sample with a second antibody, wherein the second antibody binds to a marker on the surface of a fetal cell; and (d) identifying a cell that is bound to the second antibody as a fetal cell.

In some embodiments, the fetal cell is a fetal nucleated red blood cell (fnRBC). In some embodiments, the fetal cell is a fetal trophoblast.

In some embodiments, the colloidal magnetic particles are ferrofluid magnetic particles. In some embodiments, the colloidal magnetic particle is less than 200 nm. In some embodiments, the colloidal magnetic particle is between about 80 to 200 nm. In some embodiments, the colloidal magnetic particle is between about 90 to 150 nm. In some embodiments, the colloidal magnetic particle has a magnetic mass of at least 50%. In some embodiments, the colloidal magnetic particle has a magnetic mass of at least 60%. In some embodiments, the colloidal magnetic particle has a magnetic mass of between 70% to 90%. In some embodiments, the colloidal magnetic particle comprises a crystalline core of a superparamagnetic material that is surrounded by coating molecules.

In some embodiments, the magnetic particles are coupled to a first exogenous aggregation enhancing factor (EAEF), wherein the first EAEF comprises one member of a specific binding pair selected from the group comprising biotin-streptavidin, antigen-antibody, receptor-hormone, receptor-ligand, agonist-antagonist, lectin-carbohydrate, Protein A-antibody Fc, and avidin-biotin, biotin analog-avidin, desthiobiotin-streptavidin, desthiobiotin-avidin, iminobiotin-streptavidin, and iminobiotin-avidin.

In some embodiments, step (a) comprises adding a second EAEF to increase aggregation of the magnetic particles, the second EAEF comprising the other member of the specific binding pair.

In some embodiments, step (b) comprises adding to the enriched sample a member of the specific binding pair in order to reverse aggregation of the magnetic particles in the enriched sample.

In some embodiments, the method further comprises, prior to step (a), adding to the sample at least one aggregation inhibiting agent selected from the group consisting of a reducing agent, an immune-complex, a chelating agent, and a diamino butane. In some embodiments, the aggregation inhibiting agent is a chelating agent. In some embodiments, the chelating agent is EDTA.

In some embodiments, the second antibody is an antibody that binds to TREML2 protein or comprises an antigen binding fragment that binds to a TREML2 protein.

In some embodiments, the method further comprises, prior to step (d), isolating single fetal cells. In some embodiments, isolating single fetal cells is carried out by isolating single fetal cells that are bound to the second antibody.

In some embodiments, the second antibody which is conjugated to a label. In some embodiments, the label is a fluorescent label. In some embodiments, the label is selected from phycoerythrin (PE), allophycocyanin (APC), horse radish peroxidase (HRP), and biotin.

In some embodiments, isolating single fetal cells is based on immunofluorescent technology. In some embodiments,isolating single fetal cells is carried out by fluorescence activated cell sorting (FACS). In some embodiments,isolating single cells is carried out with a DEPArray.

In some embodiments, step (d) comprises performing a sequencing analysis. In some embodiments, the sequencing analysis comprises short tandem repeat (STR) analysis.

In some embodiments, the method further comprises analyzing the fetal cell. In some embodiments, analyzing the fetal cell comprises performing a genomic or a genetic analysis. In some embodiments, performing a genetic analysis comprises detecting the presence or absence of one or more genetic abnormalities in the fetal cell.

In some embodiments, the first antibody is an antibody that binds to a TREML2 protein or comprises an antigen binding fragment that binds to a TREML2 protein.

In some embodiments, the antibody that binds to a TREML2 protein or an antigen binding fragment that binds to a TREML2 protein comprises, consists of, or consists essentially of, one or more CDRs selected from: (i) a heavy chain variable region (HCVR) complementarity determining region (CDR) 1 comprising the amino acid sequence of SEQ ID NO: 6; (ii) a HCVR CDR2 comprising the amino acid sequence of SEQ ID NO: 7; (iii) a HCVR CDR3 comprising the amino acid sequence of SEQ ID NO: 8; (iv) a light chain variable region (LCVR) CDR1 comprising the amino acid sequence of SEQ ID NO: 9; (v) a LCVR CDR2 comprising the amino acid sequence of SEQ ID NO: 10; and (vi) a LCVR CDR3 comprising the amino acid sequence of SEQ ID NO: 11. In some embodiments, the antibody that binds to a TREML2 protein or an antigen binding fragment that binds to a TREML2 protein comprises 2, 3, 4, 5 or 6 of the CDRs selected from (i)-(vi). In some embodiments, any of SEQ ID Nos: 6-11 independently comprise one or more amino acid substitutions, additions, or deletions.

In some embodiments, the antibody that binds to a TREML2 protein is selected from sc-109096, ARP49877_P050, OACA04996, AF3259, MA5-30973, PAS-47471, ABIN634968, ABIN928294, 30-552, ABIN2463297, ABIN19999041, 11655-r001, ABIN749888, bs-2737r, ABIN1999045, 11655-rp02, ABIN293207, ABIN2387613, t8282-40, ABIN4249314, nbp1-70737-20ul, and BD563661.

Further disclosed herein are anti-TREML2 antibodies. In some embodiments, the anti-TREML2 antibody, or an antigen binding fragment thereof, comprises, consists of, or consists essentially of, one or more CDRs selected from: (a) a HCVR CDR1 comprising the amino acid sequence of SEQ ID NO: 6; (b) a HCVR CDR2 comprising the amino acid sequence of SEQ ID NO: 7; (c) a HCVR CDR3 comprising the amino acid sequence of SEQ ID NO: 8; (d) a LCVR CDR1 comprising the amino acid sequence of SEQ ID NO: 9; (e) a LCVR CDR2 comprising the amino acid sequence of SEQ ID NO: 10; and (f) a LCVR CDR3 comprising the amino acid sequence of SEQ ID NO: 11. In some embodiments, any of SEQ ID Nos: 6-11 independently comprise one or more amino acid substitutions, additions, or deletions. In some embodiments, the anti-TREML2 antibody or antigen binding fragment thereof comprises two or more CDRs selected from (a)-(f). In some embodiments, wherein the anti-TREML2 antibody or antigen binding fragment thereof comprises three or more CDRs selected from (a)-(f). In some embodiments, the anti-TREML2 antibody or antigen binding fragment thereof comprises four or more CDRs selected from (a)-(f). In some embodiments, the anti-TREML2 antibody or antigen binding fragment thereof comprises five or more CDRs selected from (a)-(f). In some embodiments, the anti-TREML2 antibody or antigen binding fragment thereof comprises all of the CDRs of (a)-(f).

In some embodiments, the anti-TREML2 antibody or antigen binding fragment thereof is conjugated to a label. In some embodiments, the label is a fluorescent label. In some embodiments, the label is selected from phycoerythrin (PE), allophycocyanin (APC), horse radish peroxidase (HRP), and biotin.

In some embodiments, the anti-TREML2 antibody or antigen binding fragment thereof is conjugated to a magnetic particle. In some embodiments, the magnetic particle is a colloidal magnetic particle. In some embodiments, the colloidal magnetic particle is a ferrofluid magnetic particle. In some embodiments, the colloidal magnetic particle is less than 200 nm. In some embodiments, the colloidal magnetic particle is between about 80 to 200 nm. In some embodiments, the colloidal magnetic particle is between about 90 to 150 nm. In some embodiments, the colloidal magnetic particle has a magnetic mass of at least 50%. In some embodiments, the colloidal magnetic particle has a magnetic mass of at least 60%. In some embodiments, the colloidal magnetic particle has a magnetic mass of between 70% to 90%. In some embodiments, the colloidal magnetic particle comprises a crystalline core of a superparamagnetic material that is surrounded by coating molecules.

In some embodiments, the magnetic particle is coupled to a first exogenous aggregation enhancing factor (EAEF), wherein the first EAEF comprises one member of a specific binding pair selected from the group comprising biotin-streptavidin, antigen-antibody, receptor-hormone, receptor-ligand, agonist-antagonist, lectin-carbohydrate, Protein A-antibody Fc, and avidin-biotin, biotin analog-avidin, desthiobiotin-streptavidin, desthiobiotin-avidin, iminobiotin-streptavidin, and iminobiotin-avidin.

Disclosed herein is an anti-TREML2 antibody conjugate comprising (a) an anti-TREML2 antibody or antigen binding fragment thereof; and (b) a magnetic particle, wherein the magnetic particle is conjugated to the anti-TREML2 antibody.

In some embodiments, the magnetic particle is a colloidal magnetic particle. In some embodiments, the colloidal magnetic particle is a ferrofluid magnetic particle. In some embodiments, the colloidal magnetic particle is less than 200 nm. In some embodiments, the colloidal magnetic particle is between about 80 to 200 nm. In some embodiments, the colloidal magnetic particle is between about 90 to 150 nm. In some embodiments, the colloidal magnetic particle has a magnetic mass of at least 50%. In some embodiments, the colloidal magnetic particle has a magnetic mass of at least 60%. In some embodiments, the colloidal magnetic particle has a magnetic mass of between 70% to 90%. In some embodiments, the colloidal magnetic particle comprises a crystalline core of a superparamagnetic material that is surrounded by coating molecules.

In some embodiments, the magnetic particle is coupled to a first exogenous aggregation enhancing factor (EAEF), wherein the first EAEF comprises one member of a specific binding pair selected from the group comprising biotin-streptavidin, antigen-antibody, receptor-hormone, receptor-ligand, agonist-antagonist, lectin-carbohydrate, Protein A-antibody Fc, and avidin-biotin, biotin analog-avidin, desthiobiotin-streptavidin, desthiobiotin-avidin, iminobiotin-streptavidin, and iminobiotin-avidin.

In some embodiments, the anti-TREML2 antibody, or antigen binding fragment thereof, comprises, consists of, or consists essentially of, one or more CDRs selected from: (a) a HCVR CDR1 comprising the amino acid sequence of SEQ ID NO: 6; (b) a HCVR CDR2 comprising the amino acid sequence of SEQ ID NO: 7; (c) a HCVR CDR3 comprising the amino acid sequence of SEQ ID NO: 8; (d) a LCVR CDR1 comprising the amino acid sequence of SEQ ID NO: 9; (e) a LCVR CDR2 comprising the amino acid sequence of SEQ ID NO: 10; and (f) a LCVR CDR3 comprising the amino acid sequence of SEQ ID NO: 11. In some embodiments, any of SEQ ID Nos: 6-11 independently comprise one or more amino acid substitutions, additions, or deletions. In some embodiments, the anti-TREML2 antibody or antigen binding fragment thereof comprises, consists of, or consists essentially of, two or more CDRs selected from (a)-(f). In some embodiments, the anti-TREML2 antibody or antigen binding fragment thereof comprises, consists of, or consists essentially of, three or more CDRs selected from (a)-(f). In some embodiments, the anti-TREML2 antibody or antigen binding fragment thereof comprises, consists of, or consists essentially of, four or more CDRs selected from (a)-(f). In some embodiments, the anti-TREML2 antibody or antigen binding fragment thereof comprises, consists of, or consists essentially of five or more CDRs selected from (a)-(f). In some embodiments, the anti-TREML2 antibody or antigen binding fragment thereof comprises, consists of, or consists essentially of, all of the CDRs of (a)-(f).

In some embodiments, the anti-TREML2 antibody is selected from sc-109096, ARP49877_P050, OACA04996, AF3259, MA5-30973, PAS-47471, ABIN634968, ABIN928294, 30-552, ABIN2463297, ABIN19999041, 11655-r001, ABIN749888, bs-2737r, ABIN1999045, 11655-rp02, ABIN293207, ABIN2387613, t8282-40, ABIN4249314, nbp1-70737-20ul, and BD563661.

Further disclosed herein are kits for isolating, detecting, and/or analyzing a fetal cell. In some embodiments, the kit comprises, consists of, or consists essentially of, (a) an antibody that binds to a Triggering Receptor Expressed on Myeloid Cells Like 2 (TREML2) protein (anti-TREML2 antibody) or an antigen binding fragment thereof; and (b) a magnetic reagent comprising colloidal magnetic particles.

In some embodiments, the anti-TREML2 antibody is selected from sc-109096, ARP49877_P050, OACA04996, AF3259, MA5-30973, PAS-47471, ABIN634968, ABIN928294, 30-552, ABIN2463297, ABIN19999041, 11655-r001, ABIN749888, bs-2737r, ABIN1999045, 11655-rp02, ABIN293207, ABIN2387613, t8282-40, ABIN4249314, nbp1-70737-20ul, and BD563661.

In some embodiments, the anti-TREML2 antibody, or an antigen binding fragment thereof, comprises, consists of, or consists essentially of, one or more complementarity determining regions (CDRs) selected from: (a) a heavy chain variable region (HCVR) CDR1 comprising the amino acid sequence of SEQ ID NO: 6; (b) a HCVR CDR2 comprising the amino acid sequence of SEQ ID NO: 7; (c) a HCVR CDR3 comprising the amino acid sequence of SEQ ID NO: 8; (d) a light chain variable region (LCVR) CDR1 comprising the amino acid sequence of SEQ ID NO: 9; (e) a LCVR CDR2 comprising the amino acid sequence of SEQ ID NO: 10; and (f) a LCVR CDR3 comprising the amino acid sequence of SEQ ID NO: 11. In some embodiments, any one of SEQ ID Nos: 6-11 independently comprise one or more amino acid substitutions, additions, or deletions.

In some embodiments, the anti-TREML2 antibody or antigen binding fragment thereof is a conjugated to a label to produce a conjugated antibody. In some embodiments, the label is selected from phycoerythrin (PE), allophycocyanin (APC), horse radish peroxidase (HRP), and biotin.

In some embodiments, the colloidal magnetic particles are less than 200 nm in size. In some embodiments, the colloidal magnetic particles are ferrofluid particles.

In some embodiments, the colloidal magnetic particles are conjugated to an antibody or antigen binding fragment thereof.

In some embodiments, the antibody is an anti-TREML2 antibody. In some embodiments, the anti-TREML2 antibody, or the antigen binding fragment thereof, comprises, consists of, or consists essentially of, one or more CDRs selected from: (a) a HCVR CDR1 comprising the amino acid sequence of SEQ ID NO: 6; (b) a HCVR CDR2 comprising the amino acid sequence of SEQ ID NO: 7; (c) a HCVR CDR3 comprising the amino acid sequence of SEQ ID NO: 8; (d) a LCVR CDR1 comprising the amino acid sequence of SEQ ID NO: 9; (e) a LCVR CDR2 comprising the amino acid sequence of SEQ ID NO: 10; and (f) a LCVR CDR3 comprising the amino acid sequence of SEQ ID NO: 11. In some embodiments, any one of SEQ ID Nos: 6-11 independently comprise one or more amino acid substitutions, additions or deletions. In some embodiments, the anti-TREML2 antibody, or an antigen binding fragment thereof, comprises, consists of, or consists essentially of, 2, 3, 4, 5 or 6 of the CDRs selected from (i)-(vi).

In some embodiments, the anti-TREML2 antibody is selected from sc-109096, ARP49877_P050, OACA04996, AF3259, MA5-30973, PAS-47471, ABIN634968, ABIN928294, 30-552, ABIN2463297, ABIN19999041, 11655-r001, ABIN749888, bs-2737r, ABIN1999045, 11655-rp02, ABIN293207, ABIN2387613, t8282-40, ABIN4249314, nbp1-70737-20ul, and BD563661.

In some embodiments, the kit further comprises, consists of, or consists essentially of, an inhibiting agent selected from the group consisting of a reducing agent, an immune-complex, a chelating agent, a diamino butane. In some embodiments, the kit further comprises, consists of, or consists essentially of, a chelating agent. In some embodiments, the chelating agent is EDTA.

In some embodiments, the kit further comprises, consists of, or consists essentially of, an exogenous aggregation enhancing factor (EAEF). In some embodiments, the EAEF comprises, consists of, or consists essentially of, one member of a specific binding pair selected from the group comprising biotin-streptavidin, antigen-antibody, receptor-hormone, receptor-ligand, agonist-antagonist, lectin-carbohydrate, Protein A-antibody Fc, and avidin-biotin, biotin analog-avidin, desthiobiotin-, streptavidin, desthiobiotin-avidin, iminobiotin-streptavidin, and iminobiotin-avidin.

Further disclosed herein is kit comprising (a) a first antibody, capable of binding to a protein expressed on the surface of a fetal cell, wherein the first antibody is bound to colloidal magnetic particles; and (b) an anti-TREML2 antibody or an antigen binding fragment thereof.

In some embodiments, the first antibody binds to a protein selected from EpCAM, CD105, and CD71.

In some embodiments, the colloidal magnetic particles are ferrofluid particles

In some embodiments, the anti-TREML2 antibody, or the antigen binding fragment thereof, comprises, consists of, or consists essentially of, one or more CDRs selected from (a) a HCVR CDR1 comprising the amino acid sequence of SEQ ID NO: 6; (b) a HCVR CDR2 comprising the amino acid sequence of SEQ ID NO: 7; (c) a HCVR CDR3 comprising the amino acid sequence of SEQ ID NO: 8; (d) a LCVR CDR1 comprising the amino acid sequence of SEQ ID NO: 9; (e) a LCVR CDR2 comprising the amino acid sequence of SEQ ID NO: 10; and (f) a LCVR CDR3 comprising the amino acid sequence of SEQ ID NO: 11. In some embodiments, any one of SEQ ID Nos: 6-11 independently comprise one or more amino acid substitutions, additions or deletions.

In some embodiments, the anti-TREML2 antibody is selected from sc-109096, ARP49877_P050, OACA04996, AF3259, MA5-30973, PAS-47471, ABIN634968, ABIN928294, 30-552, ABIN2463297, ABIN19999041, 11655-r001, ABIN749888, bs-2737r, ABIN1999045, 11655-rp02, ABIN293207, ABIN2387613, t8282-40, ABIN4249314, nbp1-70737-20ul, and BD563661.

In some embodiments, the kit further comprises, consists of, or consists essentially of, an inhibiting agent selected from the group consisting of a reducing agent, an immune-complex, a chelating agent, a diamino butane. In some embodiments, the chelating agent is EDTA.

In some embodiments, the kit further comprises, consists of, or consists essentially of, an exogenous aggregation enhancing factor (EAEF), wherein the EAEF comprises, consists of, or consists essentially of, one member of a specific binding pair selected from the group comprising biotin-streptavidin, antigen-antibody, receptor-hormone, receptor-ligand, agonist-antagonist, lectin-carbohydrate, Protein A-antibody Fc, and avidin-biotin, biotin analog-avidin, desthiobiotin-streptavidin, desthiobiotin-avidin, iminobiotin-streptavidin, and iminobiotin-avidin.

### BRIEF DESCRIPTION OF THE DRAWINGS

FIG. 1 depicts an exemplary method for isolating, detecting, and analyzing rare cells.

FIG. 2 depicts an exemplary ferrofluid structure.

FIG. 3 depicts an exemplary method for detecting rare cells.

FIG. 4 depicts an exemplary method for isolating and detecting rare cells.

FIG. 5A-E depicts the gating of cells using a FACS instrument.

FIG. 6 depicts an exemplary method for isolating, detecting, and analyzing rare cells.

FIG. 7 depicts a schematic of ferrofluid aggregation via controlled aggregation.

FIG. 8: Schematic workflow for fetal cells enrichment: Fetal cells are enriched and stained from whole blood of pregnant women. Pure single cells are isolated by using of DEPArray™ for whole genome amplification and genome analysis.

FIG. 9 Gating strategy for erythroblasts isolated from a fetal blood sample: (1) FSC-A/SSC-A to gate major cell population (2) gate Sytox Green negative live cells (3) FSC-H/W to exclude doublet cells (4) gate double positive GPA/Hoechst (5) gate CD71pos/CD45neg (6) gate TLS1/TREML2 and overlay with the Isotype control to determine the % of TREML2 positive cells.

FIG. 10 Gating strategy for erythroblasts isolated from a bone marrow sample: (1) FSC-A/SSC-A to gate major cell population (2) gate Sytox Green negative live cells (3) FSC-H/W to exclude doublet cells (4) gate double positive GPA/Hoechst (5) gate CD71pos/CD45neg (6) gate TLS1/TREML2 and overlay with the Isotype control to determine the % of TREML2 positive cells.

FIGS. 11A-11J shows TLS1/TREML2 expression on fetal erythroblast isolated from various fetal blood (FB) samples from various clones.

FIGS. 12A-12L shows TLS1/TREML2 expression on adult erythroblast isolated from various bone marrow (BM) samples from various clones.

FIG. 13 shows a scatter plot analysis of TLS1/TREML-2 positive trophoblast cells identified by DEPArray™ after spiking and enrichment with CD105-FF and EpCAM-FF.

FIG. 14 shows a CellBrowser® Image gallery: Trophoblast cells shown positive staining from TREML-2-PE antibody, CK-APC and nuclear staining.

FIG. 15A shows a scatter plot analysis of Draq5/Hoechst positive erythroblasts spiked in Healthy donor blood and enriched with CD71-FF. FIG. 15B shows a CellBrowser® Image gallery: erythroblast cells shown positive staining from CD71-PE antibody, Draq5 and Hoechst nuclear staining, and negative staining for CD45-FITC antibody.

FIG. 16A shows a scatter plot analysis of Draq5/Hoechst positive erythroblasts spiked in Healthy donor blood and enriched with TLS1/TREML-2-FF. FIG. 16B shows a CellBrowser® Image gallery: erythroblast cells shown positive staining from CD71-PE antibody, Draq5 and Hoechst nuclear staining, and negative staining for CD45-FITC antibody.

FIG. 17 shows a STR Analysis from single fetal cell isolated from maternal blood.

FIG. 18 shows the results of a CNV analysis of single fetal cells.

FIG. 19 shows the results of a CNV analysis for a healthy donor single cells.

FIG. 20 depicts an exemplary method for isolating and detecting rare cells.

### DETAILED DESCRIPTION

Disclosed herein are compositions, kits, and methods for isolating, detecting, and/or analyzing rare cells in a sample. Generally, the compositions, kits, and methods disclosed herein comprise agents that bind to a Triggering Receptor Expressed on Myeloid Cells Like 2 (TREML2) protein (this protein is also referred as TLS1 throughout the application). Alternatively, or additionally, the compositions, kits, and methods disclosed herein comprise antibody conjugates. The antibody conjugates comprise an antibody conjugated to a colloidal magnetic particle. The rare cell may be a fetal cell. The sample may be a sample from a pregnant subject.

Methods for Isolating, Detecting, and/or Characterizing Rare Cells

Disclosed herein are methods for isolating, detecting, and/or characterizing rare cells. In some embodiments, the rare cells are fetal cells. In some embodiments, the fetal cells are fetal nucleated red blood cells (fnRBCs). In some embodiments, the fetal cells are trophoblasts. Generally, the methods comprise using an anti-TREML2 antibody or antigen binding fragment thereof to identify a cell as a fetal cell. Alternatively, or additionally, the methods comprise using an antibody conjugated to a colloidal magnetic particle for isolating a fetal cell.

Disclosed herein is a method for detecting fetal cells in a sample from a pregnant subject, comprising: (a) contacting the sample with an anti-TREML2 antibody or antigen binding fragment thereof, wherein the sample comprises a plurality of cells; and (b) identifying cells that are bound to the anti-TREML2 antibody as a fetal cell.

Further disclosed herein is a method for detecting fetal cells in a sample from a pregnant subject, comprising: (a) contacting the sample with a first antibody or antigen binding fragment thereof, wherein the sample comprises a plurality of cells; (b) isolating cells bound to the first antibody, or antigen binding fragment thereof, to produce an enriched sample; (c) contacting the enriched sample with a second antibody or antigen binding fragment thereof; and (d) identifying a cell that is bound to the second antibody as a fetal cell, wherein the first antibody or the second antibody is an antibody that binds to a Triggering Receptor Expressed on Myeloid Cells Like 2 (TREML2) protein.

Further disclosed herein is a method for detecting fetal cells in a sample from a pregnant subject, comprising: (a) contacting the sample with a magnetic reagent, wherein the sample comprises a plurality of cells, wherein the magnetic reagent comprises a magnetic particle conjugated to a first antibody or antigen binding fragment thereof, and wherein the first antibody, or antigen binding fragment thereof, binds to a protein selected from EpCAM, CD105, and CD71; (b) contacting the sample with an anti-TREML2 antibody or antigen binding fragment thereof; and (c) identifying a cell that is bound to the anti-TREML2 antibody as a fetal cell.

Further disclosed herein is a method for detecting fetal cells in a sample from a pregnant subject, comprising: (a) contacting the sample with a magnetic reagent, wherein the sample comprises a plurality of cells, wherein the magnetic reagent comprises a colloidal magnetic particle conjugated to a first antibody or antigen binding fragment thereof, and wherein the first antibody, or antigen binding fragment thereof, binds to a protein selected from EpCAM, CD105, and CD71; (b) contacting the sample with a second antibody or antigen binding fragment thereof; and (c) identifying a cell that is bound to the second antibody as a fetal cell.

Further disclosed herein is a method for detecting fetal cells in a sample from a pregnant subject, comprising: (a) contacting the sample with a magnetic reagent and a second exogenous aggregation enhancing factor (EAEF), wherein the sample comprises a plurality of cells, wherein the magnetic reagent comprises a colloidal magnetic particle conjugated to a first antibody or antigen binding fragment thereof, wherein the colloidal magnetic particle is conjugated to a first EAEF, and wherein the first antibody, or antigen binding fragment thereof, binds to a protein selected from EpCAM, CD105, and CD71; (b) contacting the sample with a second antibody or antigen binding fragment thereof; and (c) identifying a cell that is bound to the second antibody as a fetal cell. In some embodiments, the first EAEF comprises a first member of a specific binding pair and the second EAEF comprises a second member of the specific binding pair, wherein the specific binding pair is selected from the group comprising biotin-streptavidin, antigen-antibody, receptor-hormone, receptor-ligand, agonist-antagonist, lectin-carbohydrate, Protein A-antibody Fc, and avidin-biotin, biotin analog-avidin, desthiobiotin-streptavidin, desthiobiotin-avidin, iminobiotin-streptavidin, and iminobiotin-avidin.

Further disclosed herein is a method for detecting fetal cells in a sample from a pregnant subject, comprising: (a) contacting the sample with a first antibody conjugate, wherein the sample comprises a plurality of cells, and wherein the first antibody conjugate comprises a first antibody, or antigen binding fragment thereof, conjugated to a colloidal magnetic particle; (b) isolating cells bound to the first antibody by subjecting the sample to a magnetic field, thereby producing an enriched sample; (c) contacting the enriched sample with a second antibody or antigen binding fragment thereof, wherein the second antibody binds to a marker on the surface of a fetal cell; and (d) identifying a cell that is bound to the second antibody as a fetal cell.

Further disclosed herein is a method of preparing a fetal cell sample from a maternal sample obtained from a pregnant subject, comprising: (a) contacting the maternal sample that comprises fetal cells and maternal cells with a first antibody conjugate, wherein the first antibody conjugate comprises (i) a first antibody or antigen binding fragment thereof; and (ii) a colloidal magnetic particle, wherein the first antibody is conjugated to the colloidal magnetic particle; and (b) isolating cells that are bound to the first antibody conjugate by subjecting the maternal sample to a magnetic field, thereby preparing a fetal cell sample.

Further disclosed herein is a method of preparing a fetal cell sample from a maternal sample obtained from a pregnant subject, comprising: (a) contacting the maternal sample that comprises fetal cells and maternal cells with a first antibody conjugate and a second exogenous aggregation enhancing factor (EAEF), wherein the first antibody conjugate comprises (i) a first antibody or antigen binding fragment thereof; (ii) a colloidal magnetic particle; and (iii) a first EAEF, wherein the first antibody is conjugated to the colloidal magnetic particle, and wherein the first EAEF is conjugated to the colloidal magnetic particle; and (b) isolating cells that are bound to the first antibody conjugate by subjecting the maternal sample to a magnetic field, thereby preparing a fetal cell sample. In some embodiments, the first EAEF comprises a first member of a specific binding pair and the second EAEF comprises a second member of the specific binding pair, wherein the specific binding pair is selected from the group comprising biotin-streptavidin, antigen-antibody, receptor-hormone, receptor-ligand, agonist-antagonist, lectin-carbohydrate, Protein A-antibody Fc, and avidin-biotin, biotin analog-avidin, desthiobiotin-streptavidin, desthiobiotin-avidin, iminobiotin-streptavidin, and iminobiotin-avidin.

Further disclosed herein is a method of preparing a fetal cell sample from a maternal sample obtained from a pregnant subject, comprising: (a) contacting the maternal sample that comprises fetal cells and maternal cells with a first antibody conjugate, wherein the first antibody conjugate comprises (i) a first antibody or antigen binding fragment thereof; and (ii) a colloidal magnetic particle, wherein the first antibody is conjugated to the colloidal magnetic particle and wherein the first antibody is an anti-TREML2 antibody; and (b) isolating cells that are bound to the first antibody conjugate by subjecting the maternal sample to a magnetic field, thereby preparing a fetal cell sample.

Further disclosed herein is a method of preparing a fetal cell sample from a maternal sample obtained from a pregnant subject, comprising: (a) contacting the maternal sample that comprises fetal cells and maternal cells with a first antibody conjugate and a second exogenous aggregation enhancing factor (EAEF), wherein the first antibody conjugate comprises (i) a first antibody or antigen binding fragment thereof; and (ii) a colloidal magnetic particle, wherein the first antibody is conjugated to the colloidal magnetic particle, and wherein the colloidal magnetic particle is conjugated to a first EAEF; and (b) isolating cells that are bound to the first antibody conjugate by subjecting the maternal sample to a magnetic field, thereby preparing a fetal cell sample. In some embodiments, the first EAEF comprises a first member of a specific binding pair and the second EAEF comprises a second member of the specific binding pair, wherein the specific binding pair is selected from the group comprising biotin-streptavidin, antigen-antibody, receptor-hormone, receptor-ligand, agonist-antagonist, lectin-carbohydrate, Protein A-antibody Fc, and avidin-biotin, biotin analog-avidin, desthiobiotin-streptavidin, desthiobiotin-avidin, iminobiotin-streptavidin, and iminobiotin-avidin.

In some embodiments, the fetal cell is a fetal nucleated red blood cell (fnRBC). In some embodiments, the fetal cell is an erythroblast. In some embodiments, the fetal cell is a trophoblast.

In some embodiments, any of the methods disclosed herein further comprise isolating cells that are bound to the anti-TREML2 antibody or to the first antibody, wherein isolating the cells occurs prior to identifying cells.

In some embodiments, any of the methods disclosed herein comprise the use of a first antibody. In some embodiments, the first antibody is conjugated to one or more magnetic particles. In some embodiments, the magnetic particles are colloidal magnetic particles. In some embodiments, the magnetic particles are ferrofluid magnetic particles.

In some embodiments, any of the methods disclosed herein comprise isolating cells bound to a first antibody or antigen binding fragment thereof. In some embodiments, isolating cells comprises subjecting the sample to a magnetic field.

In some embodiments, the magnetic particles are coupled to a first exogenous aggregation enhancing factor (EAEF), the first EAEF comprising one member of a specific binding pair selected from the group comprising biotin-streptavidin, antigen-antibody, receptor-hormone, receptor-ligand, agonist-antagonist, lectin-carbohydrate, Protein A-antibody Fc, and avidin-biotin, biotin analog-avidin, desthiobiotin-streptavidin, desthiobiotin-avidin, iminobiotin-streptavidin, and iminobiotin-avidin.

In some embodiments, any of the methods disclosed herein comprise adding a second EAEF to induce aggregation of the magnetic particles, the second EAEF comprising the other member of the specific binding pair.

In some embodiments, isolating cells bound to the first antibody or antigen binding fragment thereof comprises, consists of, or consists essentially of, adding to the enriched sample a member of the specific binding pair in order to reverse aggregation of the magnetic particles in the enriched sample.

In some embodiments, any of the methods disclosed herein comprise adding to the sample at least one aggregation inhibiting agent selected from the group consisting of a reducing agent, an immune-complex, a chelating agent, and a diamino butane. In some embodiments, aggregation inhibiting agent is a chelating agent. In some embodiments, the chelating agent is EDTA. The reducing agent may be mercaptoethane sulfonic acid. The aggregation inhibitor may be a bovine serum albumin (BSA).

In some embodiments, any of the methods disclosed herein use a second antibody. In some embodiments, the second antibody is an antibody that binds to TREML2 protein or comprises, consists of, or consists essentially of, an antigen binding fragment that binds to a TREML2 protein.

In some embodiments, any of the methods disclosed herein comprise isolating single fetal cells. In some embodiments, isolating single fetal cells is carried out by isolating single fetal cells that are bound to the second antibody.

In some embodiments, the second antibody which is conjugated to a label. In some embodiments, the label is a fluorescent label. In some embodiments, isolating single fetal cells is based on immunofluorescent technology. In some embodiments, isolating single fetal cells is carried out by fluorescence activated cell sorting (FACS). In some embodiments, isolating single cells is carried out with a DEPArray.

In some embodiments, any of the methods disclosed herein comprise performing a sequencing analysis on one or more nucleic acid molecules isolated from a fetal cell. In some embodiments, the sequencing analysis comprises short tandem repeat (STR) analysis.

In some embodiments, any of the methods disclosed herein comprise analyzing a fetal cell. In some embodiments, analyzing the fetal cell comprises performing a genomic or a genetic analysis. In some embodiments, performing a genetic analysis comprises detecting the presence or absence of one or more genetic abnormalities in the fetal cell.

In some embodiments, the first antibody is an antibody that binds to a TREML2 protein or comprises an antigen binding fragment that binds to a TREML2 protein.

In some embodiments, the antibody that binds to a TREML2 protein or an antigen binding fragment that binds to a TREML2 protein comprising 1, 2, 3, 4, 5, or 6 CDRs selected from (a) a heavy chain variable region (HCVR) complementarity determining region (CDR) 1 comprising, consisting of, or consisting essentially of the amino acid sequence of SEQ ID NO: 6; (b) a HCVR CDR2 comprising, consisting of, or consisting essentially of, the amino acid sequence of SEQ ID NO: 7; (c) a HCVR CDR3 comprising, consisting of, or consisting essentially of, the amino acid sequence of SEQ ID NO: 8; (d) a light chain variable region (LCVR) CDR1 comprising, consisting of, or consisting essentially of, the amino acid sequence of SEQ ID NO: 9; (e) a LCVR CDR2 comprising, consisting of, or consisting essentially of, the amino acid sequence of SEQ ID NO: 10; and (f) a LCVR CDR3 comprising, consisting of, or consisting essentially of, the amino acid sequence of SEQ ID NO: 11. In some embodiments, any one of SEQ ID Nos: 6-11 independently comprise one or more substitutions, additions, or deletions.

In some embodiments, the anti-TREML2 antibody is conjugated to one or more magnetic particles. In some embodiments, the magnetic particles are colloidal magnetic particles. In some embodiments, the colloidal magnetic particles are ferrofluid magnetic particles. In some embodiments, isolating cells comprises placing the sample in a magnetic separator. In some embodiments, isolating cells comprises subjecting the sample to a magnetic field.

In some embodiments, the anti-TREML2 antibody is conjugated to a label. In some embodiments, the label is a fluorescent label. In some embodiments, isolating cells comprises flow cytometry. In some embodiments, flow cytometry is fluorescence activated cell sorting (FACS).

In some embodiments, isolating cells comprises performing DEPArray.

In some embodiments, identifying the cells comprises performing a sequencing reaction.

In some embodiments, the sample is a sample that is enriched for fetal cells prior to contacting the sample with the anti-TREML2 antibody. In some embodiments, the sample is enriched for fetal cells by contacting the sample with a ferrofluid reagent, wherein the ferrofluid comprises an antibody coupled to a ferrofluid.

In some embodiments, the antibody binds to a protein selected from EpCAM, CD105, and CD71.

In some embodiments, the methods disclosed herein further comprise isolating cells bound by the antibody coupled to the ferrofluid, thereby producing a sample enriched for fetal cells.

In some embodiments, any of the methods disclosed herein further comprise performing a sequencing analysis. In some embodiments, the sequencing analysis comprises short tandem repeat (STR) analysis.

In some embodiments, any of the methods disclosed herein further comprise analyzing the fetal cell. In some embodiments, analyzing the fetal cell comprises detecting the presence or absence of one or more fetal abnormalities. In some embodiments, analyzing the fetal cell comprises performing a genomic analysis. In some embodiments, analyzing the fetal cell comprises performing a genetic analysis. In some embodiments, performing a genetic analysis comprises detecting the presence or absence of one or more genetic abnormalities in the fetal cell. In some embodiments, performing a genetic analysis comprises detecting the presence or absence of a chromosomal abnormality in the fetal cell. In some embodiments, the chromosomal abnormality is trisomy 21, trisomy 18, or trisomy 13.

In some embodiments, any of the methods disclosed herein further comprise performing genetic testing on the fetal cell. In some embodiments, performing genetic testing on the fetal cell comprises detecting the presence or absence of one or more fetal abnormalities. In some embodiments, performing genetic testing on the fetal cell comprises performing a genomic analysis. In some embodiments, performing genetic testing on the fetal cell comprises performing a genetic analysis. In some embodiments, performing a genetic analysis comprises detecting the presence or absence of a chromosomal abnormality in the fetal cell. In some embodiments, the chromosomal abnormality is trisomy 21, trisomy 18, or trisomy 13.

In some embodiments, any of the methods disclosed herein further comprise providing a treatment recommendation based on the results of the analysis of the fetal cell. In some embodiments, any of the methods disclosed herein further comprise providing a treatment recommendation based on the results of genetic testing on the fetal cell.

In some embodiments, any of the methods disclosed herein further comprise administering a therapy to the subject based on the results of the analysis of the fetal cell. In some embodiments, any of the methods disclosed herein further comprise administering a therapy to the subject based on the results of genetic testing on the fetal cell.

In some embodiments, any of the methods disclosed herein further comprise recommending additional monitoring of the subject or fetus based on the results of the analysis of the fetal cell. In some embodiments, any of the methods disclosed herein further comprise recommending additional monitoring of the subject or fetus based on the results of genetic testing on the fetal cell.

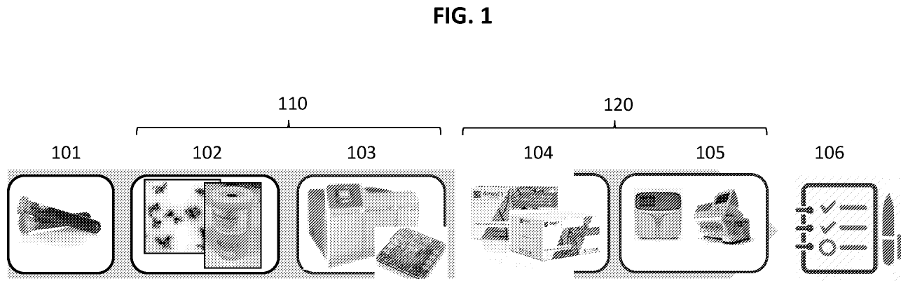

FIG. 1 depicts an exemplary method for isolating, detecting, and/or analyzing rare cells. The methods disclosed herein may comprise, consists of, or consist essentially of, one or more steps shown in FIG. 1. In some embodiments, the method comprises, consists of, or consists essentially of, (a) obtaining a sample comprising a plurality of cells from a subject (101); and (b) isolating rare cells (110). In some embodiments, the method comprises, consists of, or consists essentially of, (a) obtaining a sample comprising a plurality of cells from a subject (101); (b) isolating rare cells (110); and (c) analyzing the rare cells (120). In some embodiments, the method comprises, consists of, or consists essentially of, (a) obtaining a sample comprising a plurality of cells from a subject (101); (b) isolating rare cells (110); (c) analyzing the rare cells (120); and (d) generating or more reports based on the analysis of the rare cells (106).

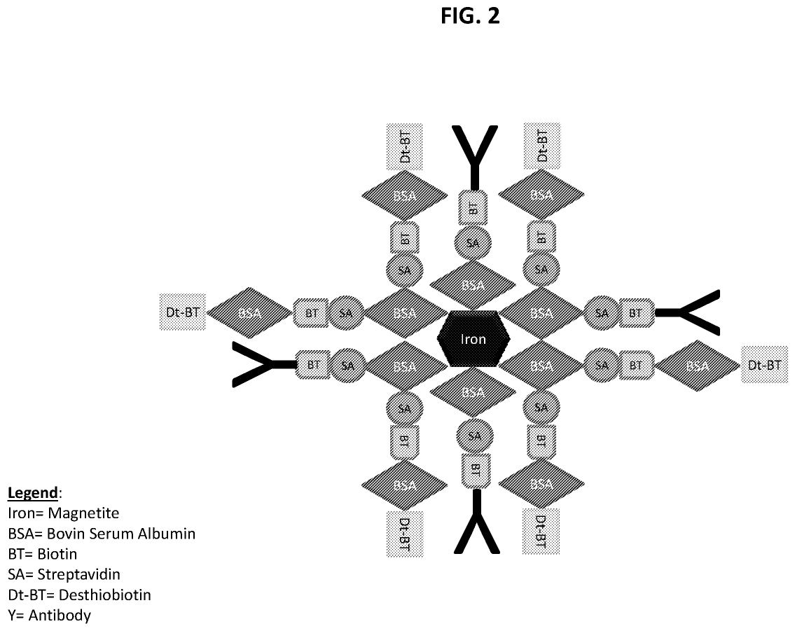

As shown in FIG. 1, in some embodiments, the method comprises, consists of, or consists essentially of, (a) obtaining a sample comprising a plurality of cells from a subject (101); (b) isolating rare cells (110) by (i) depleting non-rare cells from the sample to produce an enriched rare cell sample (102); and (ii) isolating rare cells from the enriched rare cell sample(103); (c) analyzing the rare cells (120) by (i) purifying nucleic acid molecules from the rare cells (104); and (ii) sequencing one or more nucleic acid molecules (105); and (f) generating one or more reports (106). In some embodiments, the rare cells are fetal cells. In some embodiments, enriching for rare cells (102) comprises, consists of, or consists essentially of, contacting the sample with a ferrofluid, wherein the ferrofluid comprises an antibody coupled to a magnetic particle, and wherein the antibody binds to a marker on the rare cells. In some embodiments, the marker on the rare cells is any of the markers disclosed herein. In some embodiments, the marker on the rare cells is a TREML2 protein. In some embodiments, the antibody is any of the antibodies disclosed herein. In some embodiments, the antibody is an anti-TREML2 antibody. In some embodiments, the antibody is any of the anti-TREML2 antibodies disclosed herein. In some embodiments, the ferrofluid comprises, consists of, or consists essentially of, the ferrofluid structure depicted in FIG. 2. In some embodiments, enriching for rare cells (102) further comprises, consists of, or consists essentially of, applying an external gradient magnetic separator to the sample to remove cells that are not bound to the ferrofluid. In some embodiments, the method further comprises, consists of, or consists essentially of, contacting the rare cells with one or more additional antibodies, wherein the one or more additional antibodies are conjugated to a label. In some embodiments, the label is a fluorescent label. In some embodiments, the rare cells are contacted with one or more additional antibodies prior to isolating the rare cells (103). In some embodiments, isolating rare cells (103) comprises, consists of, or consists essentially of, selecting single cells that are bound by an antibody that binds to a marker on the rare cell. In some embodiments, the antibody is an anti-TREML2 antibody. In some embodiments, the anti-TREML2 antibody is any of the anti-TREML2 antibodies disclosed herein. In some embodiments, the anti-TREML2 antibody comprises (a) a heavy chain variable region (HCVR) complementarity determining region (CDR) 1 comprising, consisting of, or consisting essentially of, the amino acid sequence of SEQ ID NO: 6; (b) a HCVR CDR2 comprising, consisting of, or consisting essentially of, the amino acid sequence of SEQ ID NO: 7; (c) a HCVR CDR3 comprising, consisting of, or consisting essentially of, the amino acid sequence of SEQ ID NO: 8; (d) a light chain variable region (LCVR) CDR1 comprising, consisting of, or consisting essentially of, the amino acid sequence of SEQ ID NO: 9; (e) a LCVR CDR2 comprising, consisting of, or consisting essentially of, the amino acid sequence of SEQ ID NO: 10; and (f) a LCVR CDR3 comprising, consisting of, or consisting essentially of, the amino acid sequence of SEQ ID NO: 11. In some embodiments, isolating rare cells (103) comprises, consists of, or consists essentially of, sorting for rare cells from an enriched cell sample. In some embodiments, isolating rare cells (103) comprises, consists of, or consists essentially of, performing fluorescence activated cell sorting (FACS). In some embodiments, isolating rare cells (103) comprises, consists of, or consists essentially of, performing DEPArray. In some embodiments, purifying nucleic acid molecules from the rare cells (104) comprises, consists of, or consists essentially of, performing a nucleic acid amplification. In some embodiments, purifying nucleic acid molecules from the rare cells (104) comprises, consists of, or consists essentially of, generating a nucleic acid library.

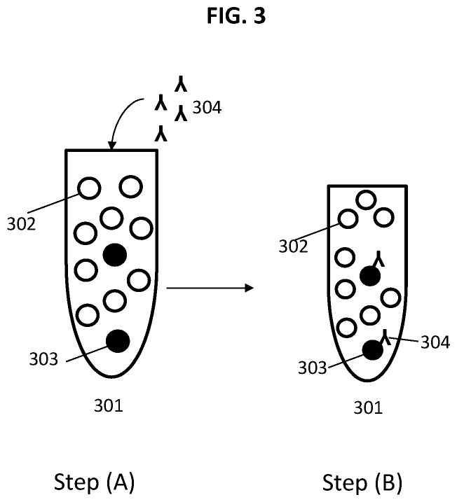

FIG. 3 depicts an exemplary method for detecting rare cells (e.g., fetal cells). In some embodiments, the method comprises, consists of, or consists essentially of, (a) contacting a sample (301) comprising a plurality of cells (302, 303) with a first antibody or antigen binding fragment thereof (304); and (b) identifying the cells (303) that are bound by the first antibody or antigen binding fragment thereof (304) as fetal cells. In some embodiments, the first antibody (304) is an antibody that binds to a TREML2 protein. In some embodiments, the antigen binding fragment (304) binds to a TREML2 protein. In some embodiments, the first antibody or antigen binding fragment thereof comprises, consists of, or consists essentially of any one of the anti-TREML2 antibodies disclosed herein. In some embodiments, the first antibody or antigen binding fragment thereof comprises, consists of, or consists essentially of, (a) a heavy chain variable region (HCVR) complementarity determining region (CDR) 1 comprising, consisting of, or consisting essentially of, the amino acid sequence of SEQ ID NO: 6; (b) a HCVR CDR2 comprising, consisting of, or consisting essentially of, the amino acid sequence of SEQ ID NO: 7; (c) a HCVR CDR3 comprising, consisting of, or consisting essentially of, the amino acid sequence of SEQ ID NO: 8; (d) a light chain variable region (LCVR) CDR1 comprising, consisting of, or consisting essentially of, the amino acid sequence of SEQ ID NO: 9; (e) a LCVR CDR2 comprising, consisting of, or consisting essentially of, the amino acid sequence of SEQ ID NO: 10; and (f) a LCVR CDR3 comprising, consisting of, or consisting essentially of, the amino acid sequence of SEQ ID NO: 11. The first antibody or antigen binding fragment thereof may be coupled to a magnetic particle. For instance, the first antibody or antigen binding fragment may be in the form of a ferrofluid. Alternatively, the first antibody or antigen binding fragment may be conjugated to a label. The label may be any of the labels disclosed herein. For instance, the first antibody or antigen binding fragment may be conjugated to a fluorescent label. The cells may be identified by any of the identification techniques disclosed herein. In some embodiments, identifying the cells that are bound to the first antibody or antigen binding fragment thereof comprises isolating the cells that are bound to the first antibody or antigen binding fragment thereof. Isolating the cells may comprise any of the cell isolation techniques disclosed herein. In some embodiments, isolating the cells comprises magnetic separation. In some embodiments, identifying the cells may comprise the use of a microscope. Identifying the cells may comprise fluorescence microscopy. In some embodiments, identifying the cells comprises or is based on FACS. Alternatively, or additionally, identifying the cells comprises or is based on a DEPArray.

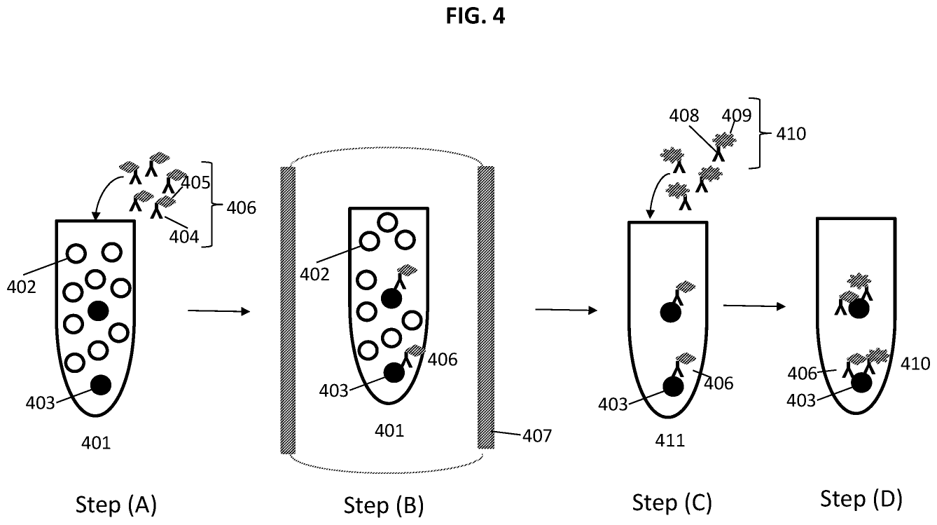

FIG. 4 depicts an exemplary method for isolating and detecting rare cells. In some embodiments, the method for detecting rare cells comprises, consists of, or consists essentially of, (a) contacting a sample (401) comprising a plurality of cells (402, 403) with a antibody conjugate (406), wherein the antibody conjugate (406) comprises a first antibody or antigen binding fragment (404) coupled to a magnetic particle (405); (b) enriching for rare cells (403) by subjecting the sample to a magnetic field (407) and removing cells (402) that are not bound to the antibody conjugate, thereby producing an enriched rare cell sample (411); (c) contacting the enriched rare cell sample (411) with an antibody conjugate (410), wherein the antibody conjugate (410) comprises a second antibody or antigen binding fragment (408) conjugated to a label (409); and (d) identifying a cell that is bound to the antibody conjugate as a rare cell (403). In some embodiments, the rare cell is a fetal cell. In some embodiments, the fetal cell is a fetal nucleated red blood cell (fnRBC). In some embodiments, the enriched rare cell sample (411) comprises the rare cell (403) bound to the antibody conjugate (406), wherein the antibody conjugate comprises the first antibody (404), or antigen binding fragment thereof (404), conjugated to the magnetic particle (405). Alternatively, or additionally, the enriched rare cell sample (411) is further processed to detach the rare cell (403) from the antibody conjugate (406). In some embodiments, the first antibody or antigen binding fragment binds to a TREML2 protein. In some embodiments, the second antibody or antigen binding fragment binds to a TREML2 protein. In some embodiments, the first antibody or antigen binding fragment and the second antibody or antigen binding fragment both bind to a TREML2 protein. In some embodiments, (a) the first antibody or antigen binding fragment or (b) the second antibody or antigen binding fragment bind to a TREML2 protein. In some embodiments, (a) the first antibody or antigen binding fragment binds to a protein selected from EpCAM, CD105, and CD71; and (b) the second antibody or antigen binding fragment binds to a TREML2 protein. In some embodiments, (a) the first antibody or antigen binding fragment binds to EpCAM; and (b) the second antibody or antigen binding fragment binds to a TREML2 protein. In some embodiments, (a) the first antibody or antigen binding fragment binds to CD105; and (b) the second antibody or antigen binding fragment binds to a TREML2 protein. In some embodiments, (a) the first antibody or antigen binding fragment binds to CD71; and (b) the second antibody or antigen binding fragment binds to a TREML2 protein. In some embodiments, the antibody or antigen binding fragment that binds to TREML2 is any of the anti-TREML2 antibodies or antigen binding fragments disclosed herein. In some embodiments, the anti-TREML2 antibody or antigen binding fragment thereof comprises, consists of, or consists essentially of, 1, 2, 3, 4, 5, or 6 CDRs selected from (a) a heavy chain variable region (HCVR) complementarity determining region (CDR) 1 comprising, consisting of, or consisting essentially of, the amino acid sequence of SEQ ID NO: 6; (b) a HCVR CDR2 comprising, consisting of, or consisting essentially of, the amino acid sequence of SEQ ID NO: 7; (c) a HCVR CDR3 comprising, consisting of, or consisting essentially of, the amino acid sequence of SEQ ID NO: 8; (d) a light chain variable region (LCVR) CDR1 comprising, consisting of, or consisting essentially of, the amino acid sequence of SEQ ID NO: 9; (e) a LCVR CDR2 comprising, consisting of, or consisting essentially of, the amino acid sequence of SEQ ID NO: 10; and (f) a LCVR CDR3 comprising, consisting of, or consisting essentially of, the amino acid sequence of SEQ ID NO: 11. In some embodiments, the second antibody is an antibody that binds to the first antibody. For instance, if the first antibody is a goat IgG antibody, the second antibody may be a mouse anti-goat IgG antibody. In some embodiments, the label is any one of the labels disclosed herein. In some embodiments, the label is a fluorescent label. In some embodiments, the magnetic particle is a colloidal magnetic particle. In some embodiments, the colloidal magnetic particle is a ferrofluid magnetic particle. In some embodiments, the magnetic particle is further conjugated to a first exogenous aggregation enhancing factor (EAEF). In some embodiments, the method further comprises, during step (A), contacting the sample (401) with a second EAEF that is capable of binding to the first EAEF. In some embodiments, the addition of the second EAEF induces aggregation of the antibody conjugate (406). In some embodiments, the method further comprises adding a third EAEF that is capable of binding to the first or second exogenous aggregation enhancing factors. In some embodiments, the addition of the third EAEF reverses aggregation of the first EAEF. In some embodiments, the method further comprises, prior to step (a), adding an aggregation inhibiting agent to the sample. The cells may be identified by any of the identification techniques disclosed herein. In some embodiments, identifying the cell may comprise the use of a microscope. Identifying the cell may comprise fluorescence microscopy. In some embodiments, identifying the cell comprises or is based on FACS. Alternatively, or additionally, identifying the cell comprises or is based on DEPArray.

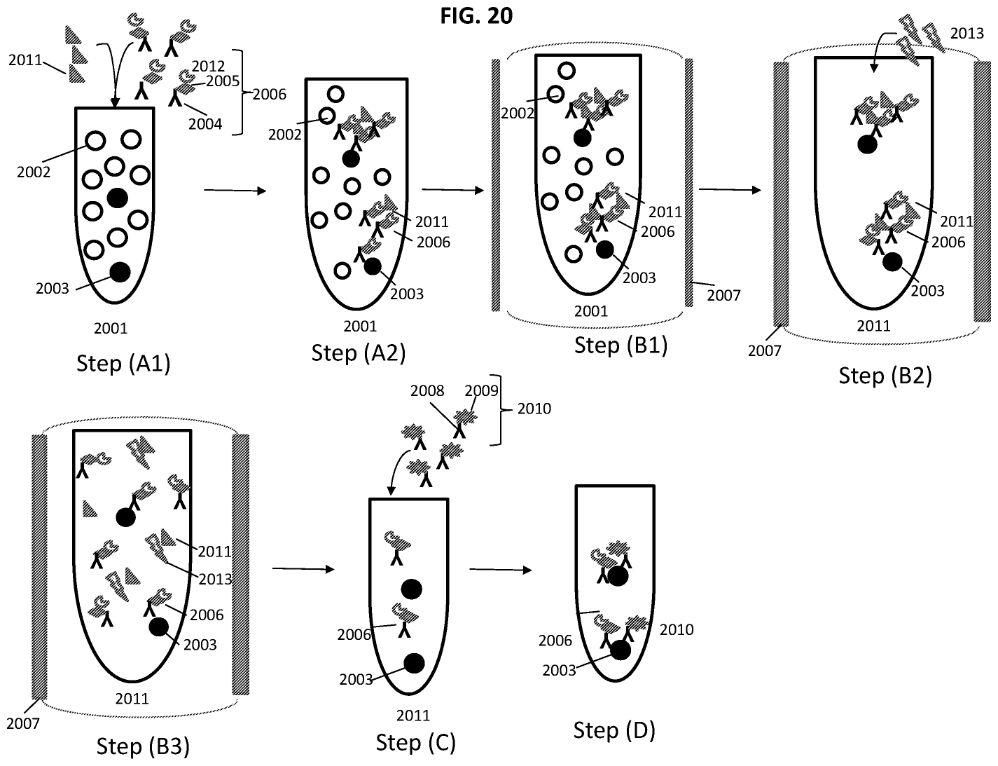

FIG. 20 depicts another exemplary method for isolating and detecting rare cells. As shown in FIG. 20, in some embodiments, the method for detecting rare cells comprises, consists of, or consists essentially of, step (A1): contacting a sample (2001) comprising a plurality of cells (2002, 2003) with a first antibody conjugate (2006) and a second exogenous aggregation enhancing factor (EAEF) (2011), wherein the first antibody conjugate (2006) comprises a first antibody or antigen binding fragment (2004) coupled to a magnetic particle (2005), wherein the magnetic particle (2005) is further conjugated to a first EAEF (2012); and step (B1) enriching for rare cells (2003) by subjecting the sample to a magnetic field (2007) and removing cells (2002) that are not bound to the antibody conjugate (2006)-second EAEF (2011i) complex, thereby producing an enriched rare cell sample (2011). As shown in step (A2), the addition of the second EAEF (2011) induces aggregation of the first antibody conjugate (2006). In some embodiments, the method further comprises step (B2) adding a third EAEF (2013) to the enriched rare cell sample (2011). As shown in step (B3), the addition of the third EAEF (2013) reverses aggregation of the first antibody conjugate (2006). In some embodiments, the method further comprises step (C) contacting the enriched rare cell sample (2011) with a second antibody conjugate (2010), wherein the second antibody conjugate (2010) comprises a second antibody or antigen binding fragment (2008) conjugated to a label (2009). In some embodiments, the method further comprises step (D) identifying a cell that is bound to the first antibody conjugate (2006) as a rare cell (2003). In some embodiments, the method further comprises step (D) identifying a cell that is bound to the second antibody conjugate (2010) as a rare cell (2003). In some embodiments, the rare cell is a fetal cell. In some embodiments, the fetal cell is a fetal nucleated red blood cell (fnRBC). In some embodiments, the enriched rare cell sample (2011) comprises the rare cell (2003) bound to the first antibody conjugate (2006), wherein the first antibody conjugate comprises the first antibody (2004), or antigen binding fragment thereof (2004), conjugated to the magnetic particle (2005), wherein the magnetic particle (2005) is further conjugated to the first EAEF (2012). Alternatively, or additionally, the enriched rare cell sample (2011) is further processed to detach the rare cell (2003) from the antibody conjugate (2006). In some embodiments, the first antibody or antigen binding fragment binds to a TREML2 protein. In some embodiments, the second antibody or antigen binding fragment binds to a TREML2 protein. In some embodiments, the first antibody or antigen binding fragment and the second antibody or antigen binding fragment both bind to a TREML2 protein. In some embodiments, (a) the first antibody or antigen binding fragment or (b) the second antibody or antigen binding fragment bind to a TREML2 protein. In some embodiments, (a) the first antibody or antigen binding fragment binds to a protein selected from EpCAM, CD105, and CD71; and (b) the second antibody or antigen binding fragment binds to a TREML2 protein. In some embodiments, (a) the first antibody or antigen binding fragment binds to EpCAM; and (b) the second antibody or antigen binding fragment binds to a TREML2 protein. In some embodiments, (a) the first antibody or antigen binding fragment binds to CD105; and (b) the second antibody or antigen binding fragment binds to a TREML2 protein. In some embodiments, (a) the first antibody or antigen binding fragment binds to CD71; and (b) the second antibody or antigen binding fragment binds to a TREML2 protein. In some embodiments, the antibody or antigen binding fragment that binds to TREML2 is any of the anti-TREML2 antibodies or antigen binding fragments disclosed herein. In some embodiments, the anti-TREML2 antibody or antigen binding fragment thereof comprises, consists of, or consists essentially of, 1, 2, 3, 4, 5, or 6 CDRs selected from (a) a heavy chain variable region (HCVR) complementarity determining region (CDR) 1 comprising, consisting of, or consisting essentially of, the amino acid sequence of SEQ ID NO: 6; (b) a HCVR CDR2 comprising, consisting of, or consisting essentially of, the amino acid sequence of SEQ ID NO: 7; (c) a HCVR CDR3 comprising, consisting of, or consisting essentially of, the amino acid sequence of SEQ ID NO: 8; (d) a light chain variable region (LCVR) CDR1 comprising, consisting of, or consisting essentially of, the amino acid sequence of SEQ ID NO: 9; (e) a LCVR CDR2 comprising, consisting of, or consisting essentially of, the amino acid sequence of SEQ ID NO: 10; and (f) a LCVR CDR3 comprising, consisting of, or consisting essentially of, the amino acid sequence of SEQ ID NO: 11. In some embodiments, the second antibody is an antibody that binds to the first antibody. For instance, if the first antibody is a goat IgG antibody, the second antibody may be a mouse anti-goat IgG antibody. In some embodiments, the label is any one of the labels disclosed herein. In some embodiments, the label is a fluorescent label. In some embodiments, the magnetic particle is a colloidal magnetic particle. In some embodiments, the colloidal magnetic particle is a ferrofluid magnetic particle. In some embodiments, the method further comprises, prior to step (A1), adding an aggregation inhibiting agent to the sample. In some embodiments, the first EAEF (2012) is desthiobiotin. In some embodiments, the second EAEF (2011) is streptavidin. In some embodiments, the third EAEF (2013) is biotin. The cells may be identified by any of the identification techniques disclosed herein. In some embodiments, identifying the cell may comprise the use of a microscope. Identifying the cell may comprise fluorescence microscopy. In some embodiments, identifying the cell comprises or is based on FACS. Alternatively, or additionally, identifying the cell comprises or is based on DEPArray. In some embodiments, identifying the cell comprises or is based on an immune-based assay.

Although the methods disclosed herein may recite the use of an anti-TREML2 antibody, or antigen binding fragment thereof, or antibody conjugates comprising the anti-TREML2 antibody, any of these methods may be performed by using any agent that can bind to a TREML2 protein or conjugates comprising an agent that can bind to a TREML2 protein. Accordingly, the methods disclosed herein are not limited to the use of an anti-TREML2 antibody, or antigen binding fragment thereof, or antibody conjugates comprising the anti-TREML2 antibody.

Methods for Cell-Based Fetal Genetic Testing

The identification of a novel fetal cell marker, such as TREML-2, allows for the isolation and/or detection of fetal cells and the subsequent analysis of such cells. Accordingly, disclosed herein are methods for cell-based fetal genetic testing. In some embodiments, the methods comprise (a) using an anti-TREML2 antibody to isolate fetal cells from a sample from a pregnant subject; and (b) analyzing one or more nucleic acid molecules from the fetal cells to determine the likelihood of the fetus having one or more genetic abnormalities. Alternatively, the methods comprise isolating fetal cells using any of the methods for isolating or detecting fetal cells disclosed herein and analyzing one or more nucleic acid molecules from the isolated or detected fetal cells to determine the likelihood of the fetus having one or more genetic abnormalities. In some embodiments, the methods comprise analyzing fetal cells that are isolated and/or detected by any of the methods disclosed herein. In some embodiments, the methods comprise analyzing fetal cells that are prepared by any of the methods disclosed herein.

Disclosed herein is a method for cell-based fetal genetic testing, comprising: (a) contacting a sample obtained from a pregnant subject with an anti-TREML2 antibody or antigen binding fragment thereof, wherein the sample comprises a plurality of cells; (b) isolating cells that are bound to the anti-TREML2 antibody or antigen binding fragment thereof; (c) analyzing one or more nucleic acid molecules from the cells that are bound to the anti-TREML2 antibody or antigen binding fragment thereof; and (d) generating a report based on the analysis of the one or more nucleic acid molecules, wherein the report provides the likelihood of a fetus having one or more genetic abnormalities.

Disclosed herein is a method for cell-based fetal genetic testing, comprising: (a) contacting a sample obtained from a pregnant subject with a first antibody or antigen binding fragment thereof, wherein the sample comprises a plurality of cells, wherein the first antibody or antigen binding fragment is conjugated to a colloidal magnetic particle, and wherein the first antibody or antigen binding fragment thereof binds to a marker on a fetal cell; (b) isolating cells that are bound to the first antibody or antigen binding fragment thereof; (c) analyzing one or more nucleic acid molecules from the cells that are bound to the first antibody or antigen binding fragment thereof; and (d) generating a report based on the analysis of the one or more nucleic acid molecules, wherein the report provides the likelihood of a fetus having one or more genetic abnormalities.

Disclosed herein is a method for cell-based fetal genetic testing, comprising: (a) contacting a sample obtained from a pregnant subject with a first antibody or antigen binding fragment thereof and a second exogenous aggregation enhancing factor (EAEF), wherein the sample comprises a plurality of cells, wherein the first antibody or antigen binding fragment is conjugated to a colloidal magnetic particle, wherein the colloidal magnetic particle is conjugated to a first EAEF, and wherein the first antibody or antigen binding fragment thereof binds to a marker on a fetal cell; (b) isolating cells that are bound to the first antibody or antigen binding fragment thereof; (c) analyzing one or more nucleic acid molecules from the cells that are bound to the first antibody or antigen binding fragment thereof; and (d) generating a report based on the analysis of the one or more nucleic acid molecules, wherein the report provides the likelihood of a fetus having one or more genetic abnormalities. In some embodiments, the first EAEF comprises a first member of a specific binding pair and the second EAEF comprises a second member of the specific binding pair, wherein the specific binding pair is selected from the group comprising biotin-streptavidin, antigen-antibody, receptor-hormone, receptor-ligand, agonist-antagonist, lectin-carbohydrate, Protein A-antibody Fc, and avidin-biotin, biotin analog-avidin, desthiobiotin-streptavidin, desthiobiotin-avidin, iminobiotin-streptavidin, and iminobiotin-avidin.

In some embodiments, the first antibody is an anti-TREML2 antibody. In some embodiments, the first antibody is an anti-CD71 antibody. In some embodiments, the first antibody is an anti-EpCAM antibody. In some embodiments, the first antibody is an anti-CD105 antibody. In some embodiments, when the first antibody is an anti-ECAM antibody or an anti-CD105 antibody, the method further comprises contact the isolated cells with a second antibody or antigen binding fragment thereof, wherein the second antibody binds to a marker on the fetal cell. In some embodiments, the second antibody is an anti-TREML2 antibody. In some embodiments, the second antibody is an anti-CD71 antibody. In some embodiments, the second antibody or antigen binding fragment thereof is conjugated to a label. In some embodiments, the label is a fluorescent label. In some embodiments, the method further comprises isolating cells that are bound to the second antibody. In some embodiments, the method further comprises analyzing nucleic acid molecules from the cells that are bound to the second antibody or antigen binding fragment thereof.

In some embodiments, the cells that are bound to the anti-TREML2 antibody or antigen binding fragment thereof are fetal cells. In some embodiments, the fetal cells are fetal erythroblasts. In some embodiments, the fetal cells are fetal nucleated red blood cells (fnRBCs). In some embodiments, the fetal cells are fetal trophoblasts.

In some embodiments, analyzing the one or more nucleic acid molecules comprised conducting a karyotype analysis. Karyotype analysis may be performed using any of the techniques known in the art.

In some embodiments, analyzing the one or more nucleic acid molecules comprises performing a sequencing analysis. Sequencing analysis may be performed using any of the techniques known in the art. In some embodiments, the sequencing analysis comprises short tandem repeat (STR) analysis.

In some embodiments, analyzing the one or more nucleic acid molecules comprises performing one or more amplification reactions. Nucleic acid amplification may be performed by any of the techniques known in the art. In some embodiments, the nucleic acid amplification is performed by polymerase chain reaction (PCR).

In some embodiments, the one or more genetic abnormalities is selected from a trisomy, sex chromosome anomaly, and structural anomaly. In some embodiments, the genetic abnormality is a trisomy. In some embodiments, the trisomy is selected from trisomy 3, trisomy 4, trisomy 6, trisomy 7, trisomy 8, trisomy 9, trisomy 10, trisomy 11, trisomy 12, trisomy 13, trisomy 16, trisomy 17, trisomy 18, trisomy 20, trisomy 21, and trisomy 22. In some embodiments, the genetic abnormality is a sex chromosome anomaly. In some embodiments, the sex chromosome anomaly is selected from monosomy X, triple X, and Klinefelter's syndrome. In some embodiments, the genetic abnormality is a structural anomaly. In some embodiments, the structural anomaly is a copy number variation (CNV). In some embodiments, the structural anomaly is a deletion of the CNV or duplication of the CNV.

In some embodiments, the anti-TREML2 antibody or antigen binding fragment thereof is conjugated to a magnetic particle. In some embodiments, the magnetic particle is a colloidal magnetic particle.

In some embodiments, isolating cells that are bound to the anti-TREML2 antibody or antigen binding fragment thereof comprises subjecting the sample to a magnetic field.

In some embodiments, the methods disclosed herein further comprise, prior to contacting the sample with the anti-TREML2 antibody, contacting the sample with a first antibody, wherein the first antibody binds to a protein selected from EpCAM, CD105, and CD71. In some embodiments, the methods disclosed herein further comprise, prior to contacting the sample with the anti-TREML2 antibody, isolating cells that are bound to the first antibody. In some embodiments, the first antibody is conjugated to a magnetic particle. In some embodiments, the magnetic particle is a colloidal magnetic particle. In some embodiments, isolating cells that are bound to the first antibody comprises subjecting the sample to a magnetic field.

In some embodiments, the anti-TREML2 antibody or antigen binding fragment thereof is conjugated to a label. In some embodiments, the label is a fluorescent label. In some embodiments, isolating cells that are bound to the anti-TREML2 antibody or antigen binding fragment thereof is based on immunofluorescent technology. In some embodiments, isolating cells that are bound to the anti-TREML2 antibody or antigen binding fragment thereof is carried out by fluorescence activated cell sorting (FACS). In some embodiments, isolating cells that are bound to the anti-TREML2 antibody or antigen binding fragment thereof is carried out with a DEPArray.

In some embodiments, the methods disclosed herein further comprise contacting the cells that are bound to the anti-TREML2 antibody or antigen binding fragment thereof with a second antibody or antigen binding fragment thereof. In some embodiments, second antibody is an anti-TREML2 antibody or antigen binding fragment thereof. In some embodiments, the second antibody is conjugated to a label. In some embodiments, the label is a fluorescent label. In some embodiments, the methods disclosed herein further comprise isolating cells that are bound to the second antibody or antigen binding fragment thereof. In some embodiments, isolating cells that are bound to the second antibody or antigen binding fragment thereof is based on immunofluorescent technology. In some embodiments, isolating cells that are bound to the second antibody or antigen binding fragment thereof is carried out by fluorescence activated cell sorting (FACS). In some embodiments, isolating cells that are bound to the second antibody or antigen binding fragment thereof is carried out with a DEPArray.

In some embodiments, the anti-TREML2 antibody is selected from sc-109096, ARP49877_P050, OACA04996, AF3259, MA5-30973, PAS-47471, ABIN634968, ABIN928294, 30-552, ABIN2463297, ABIN19999041, 11655-r001, ABIN749888, bs-2737r, ABIN1999045, 11655-rp02, ABIN293207, ABIN2387613, t8282-40, ABIN4249314, nbp1-70737-20ul, and BD563661. Alternatively, or additionally, the anti-TREML2 antibody, or an antigen binding fragment thereof, comprises, consists of, or consists essentially of, 1, 2, 3, 4, 5, or 6 CDRs selected from (a) a heavy chain variable region (HCVR) complementarity determining region (CDR) 1 comprising, consisting of, or consisting essentially of, the amino acid sequence of SEQ ID NO: 6; (b) a HCVR CDR2 comprising, consisting of, or consisting essentially of, the amino acid sequence of SEQ ID NO: 7; (c) a HCVR CDR3 comprising, consisting of, or consisting essentially of, the amino acid sequence of SEQ ID NO: 8; (d) a light chain variable region (LCVR) CDR1 comprising, consisting of, or consisting essentially of, the amino acid sequence of SEQ ID NO: 9; (e) a LCVR CDR2 comprising, consisting of, or consisting essentially of, the amino acid sequence of SEQ ID NO: 10; and (f) a LCVR CDR3 comprising, consisting of, or consisting essentially of, the amino acid sequence of SEQ ID NO: 11. In some embodiments, any one of SEQ ID Nos: 6-11 independently comprise one or more amino acid substitutions, additions or deletions. In some embodiments, any one of SEQ ID Nos: 6-11 independently comprise two or more amino acid substitutions, additions or deletions.

In some embodiments, any of the methods disclosed herein further comprise providing a treatment recommendation based on the results of genetic testing on the fetal cell.

In some embodiments, any of the methods disclosed herein further comprise administering a therapy to the subject based on the results of genetic testing on the fetal cell.

In some embodiments, any of the methods disclosed herein further comprise recommending additional monitoring of the subject or fetus based on the results of genetic testing on the fetal cell.

Although the methods disclosed herein may recite the use of an anti-TREML2 antibody, or antigen binding fragment thereof, or antibody conjugates comprising the anti-TREML2 antibody, any of these methods may be performed by using any agent that can bind to a TREML2 protein or conjugates comprising an agent that can bind to a TREML2 protein. Accordingly, the methods disclosed herein are not limited to the use of an anti-TREML2 antibody, or antigen binding fragment thereof, or antibody conjugates comprising the anti-TREML2 antibody.

Agents that Bind a Rare Cell Marker

Disclosed herein are agents that bind a rare cell marker. As used herein, a “rare cell marker” is a marker (e.g., cell surface protein) on a rare cell (e.g., a fetal cell). The rare cell marker may be a cell surface protein that is expressed at a higher level on a rare cell than another type of cell in a sample. The rare cell marker may be a Triggering Receptor Expressed on Myeloid Cells Like 2 (TREML2) protein. The rare cell marker may be a human TREML2 protein. The human TREML2 protein may have the amino acid sequence of SEQ ID NO: 1. Alternatively, the rare cell marker may be CD71. In some embodiments, the rare cell marker is not CD71.

As used herein, the terms “TREML2” and “TLS1” refer to the same protein and are used interchangeably). TLS1 and TREML2 refer to the same marker having identical sequences corresponding to the amino acid sequence of SEQ ID NO: 1 and comprising the domains and fragments having the amino acid sequences of SEQ ID Nos: 2-5.

As used herein, a “rare cell” refers to a cell that is present in a sample from a subject at a concentration of less than 10% of the total cell population, wherein the sample is a non-purified or non-enriched sample. In some embodiments, the rare cell is present in the sample at a concentration of less than 9%, 8%, 7%, 6%, 5%, 4%, 3%, 2%, or 1% of the total cell population. In some embodiments, the rare cell is present in the sample at a concentration of less than 1% of the total cell population. In some embodiments, the rare cell is a fetal cell and the sample is from a pregnant subject.

As used herein, the terms “non-purified sample” or “non-enriched sample” can be used interchangeably and refer to a sample that is obtained from a subject that has not been processed in a way that removes or isolates cells from the sample. Alternatively, or additionally, a non-purified sample or non-enriched sample refers to a sample obtained from a subject that has not been depleted of one or more cells. Alternatively, or additionally, a non-purified or non-enriched sample refers to a sample obtained from a subject that contains a plurality of different cell types.

In some embodiments, an agent that binds a rare cell marker is selected from an antibody, antibody fragment, receptor, and ligand. In some embodiments, the antibody fragment comprises the antigen binding domain of an antibody. In some embodiments, the antibody fragment is selected from a monovalent antigen binding fragment (Fab or Fab′), a divalent antigen binding fragment ((Fab)2 or (Fab′)2), a variable fragment (Fv), a single chain variable fragment (scFv), a bivalent diabody, a triabody, a tetrabody, minibody, and a bispecific scFv (bis-scFv).

Generally, monovalent Fab fragments have one antigen binding site, whereas divalent (Fab)2 fragments have two antigen binding regions that are linked by disulfide bonds. Fab fragments consist of the heavy chain variable (V_{H}) and light chain variable (V_{L}) regions and the heavy chain 1 constant (C_{H}¹) and light chain 1 (C_{L}¹) constant regions of an antibody. Fv fragments have the antigen binding, site made of the heavy chain variable (V_{H}) and light chain variable (V_{L}) regions, but lack the constant regions of Fab (C_{H}1 and C_{L}) regions. The V_{H} and V_{L} are held together in Fv fragments by non-covalent interactions. The Fab may be a dimer (Fab₂) or trimer (Fab₃), which allows for the binding of 2 or 3 different antigens, respectively.

The orientation of the V-domains and the linker length can be varied to create different forms of Fv molecules. Generally, when the linker is at least 12 residues long, the scFv fragments are primarily monomeric. Linkers that are 3-11 residues long yield scFv molecules that are unable to fold into a functional Fv domain. These molecules associate with a second scFv molecule, which creates a bivalent diabody. Triabodies or tetrabodies may be formed if the linker length is less than three residues. Minibodies are scFv-C_{H}3 fusion proteins that assemble into bivalent dimers. Bis-scFv fragments consists of scFv fragments with two different variable domains and are capable of binding two different epitopes concurrently.

The antibody may be a polyclonal antibody. Alternatively, or additionally, the antibody may be a monoclonal antibody. The antibody may be an imnunoglobulin gamma (IgG) antibody. The IgG antibody may be an IgG1 antibody. The IgG antibody may be an IgG2 antibody. The IgG antibody may be an IgG3 antibody. The IgG antibody may be an IgG4 antibody. The antibody may be an immunoglobulin mu (IgM) antibody. The antibody may be an immunoglobulin epsilon (IgE) antibody. The antibody may be an immunoglobulm delta (IgD) antibody. The antibody may be an immunoglobulin alpha (IgA) antibody. The IgGA antibody may be an IgGA1 antibody. Alternatively, the igG antibody is an IgGA2 antibody.

In some embodiments, the agent is an antibody or antibody fragment that binds to a TREML2 protein. In some embodiments, the antibody or antibody fragment binds to the extracellular domain of the TREML2 protein. In some embodiments, the extracellular domain has the amino acid sequence of SEQ ID NO: 2. Alternatively, the antibody or antibody fragment may bind to a fragment of the extracellular domain of TREML2. The fragment of the extracellular domain has the amino acid sequence of SEQ ID NO: 3-4. The antibody or antibody fragment may bind to the N-terminal domain of the TREML2 protein.

In some embodiments, the anti-TREML2 antibody is a polyclonal antibody. The polyclonal antibody may be selected from anti-TREML2 antibody is selected from sc-109096 (Santa Cruz Biotechnology, Inc.), ARP49877_P050 (Aviva Systems Biology), OACA04996 (Aviva Systems Biology), AF3259 (R&D Systems), PAS-47471 (Thermo Fisher), ABIN634968 (Antibodies-online. com), ABIN928294 (Antibodies-online. com), 30-552 (ProSci), ABIN2463297 (antibodies-online.com), ABIN749888 (antibodies-online.com), bs-2737r (Bioss), ABIN1999045 (antibodies-online.com), 11655-rp02 (Sino Biological), ABIN293207 (antibodies-online. com), ABIN23 87613 (antibodies-online. com), t8282-40 (USBio), ABIN4249314 (antibodies-online.com), and nbp1-70737-20ul (Novus Biologicals).

The anti-TREML2 antibody may be a monoclonal antibody. The monoclonal antibody may be selected from MA5-30973 (Thermo Fisher), ABIN19999041 (antibodies-online.com), 11655-r001 (Sino Biological), and BD563661 (Fisher Scientific).

In some embodiments, the anti-TREML2 antibody or antigen binding fragment thereof comprises, consists of, or consists essentially of, 1, 2, 3, 4, 5, or 6 CDRs selected from (a) a heavy chain variable region (HCVR) complementarity determining region (CDR) 1 comprising, consisting of, or consisting essentially of, the amino acid sequence of SEQ ID NO: 6; (b) a HCVR CDR2 comprising, consisting of, or consisting essentially of, the amino acid sequence of SEQ ID NO: 7; (c) a HCVR CDR3 comprising, consisting of, or consisting essentially of, the amino acid sequence of SEQ ID NO: 8; (d) a light chain variable region (LCVR) CDR1 comprising, consisting of, or consisting essentially of, the amino acid sequence of SEQ ID NO: 9; (e) a LCVR CDR2 comprising, consisting of, or consisting essentially of, the amino acid sequence of SEQ ID NO: 10; and (f) a LCVR CDR3 comprising, consisting of, or consisting essentially of, the amino acid sequence of SEQ ID NO: 11.

In some embodiments, the anti-TREML2 antibody or antigen binding fragment thereof comprises, consists of, or consists essentially of, 1, 2, or 3 CDRs selected from (a) a heavy chain variable region (HCVR) complementarity determining region (CDR) 1 comprising, consisting of, or consisting essentially of, the amino acid sequence of SEQ ID NO: 6; (b) a HCVR CDR2 comprising, consisting of, or consisting essentially of, the amino acid sequence of SEQ ID NO: 7; and (c) a HCVR CDR3 comprising, consisting of, or consisting essentially of, the amino acid sequence of SEQ ID NO: 8.

In some embodiments, the anti-TREML2 antibody or antigen binding fragment thereof comprises, consists of, or consists essentially of, 1, 2, or 3 CDRs selected from (a) a light chain variable region (LCVR) CDR1 comprising, consisting of, or consisting essentially of, the amino acid sequence of SEQ ID NO: 9; (b) a LCVR CDR2 comprising, consisting of, or consisting essentially of, the amino acid sequence of SEQ ID NO: 10; and (c) a LCVR CDR3 comprising, consisting of, or consisting essentially of, the amino acid sequence of SEQ ID NO: 11.

In some embodiments, the anti-TREML2 antibody or antigen binding fragment thereof comprises, consists of, or consists essentially of, (a) a heavy chain variable region (HCVR) complementarity determining region (CDR) 1 comprising, consisting of, or consisting essentially of, the amino acid sequence of SEQ ID NO: 6; (b) a HCVR CDR2 comprising, consisting of, or consisting essentially of, the amino acid sequence of SEQ ID NO: 7; (c) a HCVR CDR3 comprising, consisting of, or consisting essentially of, the amino acid sequence of SEQ ID NO: 8; (d) a light chain variable region (LCVR) CDR1 comprising, consisting of, or consisting essentially of, the amino acid sequence of SEQ ID NO: 9; and (e) a LCVR CDR2 comprising, consisting of, or consisting essentially of, the amino acid sequence of SEQ ID NO: 10; and (f) a LCVR CDR3 comprising, consisting of, or consisting essentially of, the amino acid sequence of SEQ ID NO: 11.

In some embodiments, any one of SEQ ID Nos: 6-11 independently comprise one, two, or three or more amino acid substitutions, additions or deletions. In some embodiments, SEQ ID NO: 6 comprises one, two, or three or more amino acid substitutions, additions or deletions. In some embodiments, SEQ ID NO: 7 comprises one, two, or three or more amino acid substitutions, additions or deletions. In some embodiments, SEQ ID NO: 8 comprises one, two, or three or more amino acid substitutions, additions or deletions. In some embodiments, SEQ ID NO: 9 comprises one, two, or three or more amino acid substitutions, additions or deletions. In some embodiments, SEQ ID NO: 10 comprises one amino acid substitutions, additions or deletions. In some embodiments, SEQ ID NO: 11 comprises one, two, or three or more amino acid substitutions, additions or deletions.

In some embodiments, the anti-TREML2 antibody is a conjugated a label to produce a conjugated antibody. In some embodiments, the label is selected from a fluorescent label, a radionuclide, an enzymatic label, a chemiluminescent label, and a hapten. In some embodiments, the detectable label is a hapten. In some embodiments, the hapten is selected from DCC, biotin, nitropyrazole, thiazolesulfonamide, benzofurazan, and 2-hydroxyquinoxaline. In some embodiments, the detectable label is biotin. In some embodiments, the label is a fluorescent molecule. In some embodiments, the fluorescent molecule is selected from a fluorophore, a cyanine dye, and a near infrared (NIR) dye. In some embodiments, the fluorescent molecule is fluorescein. In some embodiments, the fluorescent molecule is fluorescein isothiocyanate (FITC). In some embodiments, the label is selected from phycoerythrin (PE), allophycocyanin (APC), horse radish peroxidase (HRP), and biotin. In some embodiments, the conjugated antibody is selected from ABIN6070559 (antibodies-online.com), abx307664 (Abbexa polyclonal), ABIN6070561 (antibodies-online.com), abx307665 (Abbexa, polyclonal), ABIN2662892 (antibodies-online.com), bld-351203 (BioLegend), ABIN2662891 (antibodies-online.com), bld-351204 (BioLegend), ABIN2662890 (antibodies-online.com, monoclonal), and bld-351104 (BioLegend).

Magnetic Particles

The methods, compositions, and kits disclosed herein may comprise or use magnetic particles. For instance, any of the antibodies (or more in general any agents that bind the rare cell marker) disclosed herein may be conjugated to a magnetic particle. In some embodiments, an agent that binds to a rare cell maker (e.g., TREML2) is conjugated to a magnetic particle. The magnetic particles may be colloidal magnetic particles. The colloidal magnetic particles may be ferrofluids.

As used herein, the term “magnetic particle” refers to a particle that can be manipulated using a magnetic field. A magnetic particle comprises a metal. Examples of metals include, but are not limited to, iron, nickel, cobalt, and copper.

As used herein, the term “colloidal magnetic particle” refers to a magnetic particle that is coated with a non-magnetic material. An example of a non-magnetic particle is bovine serum albumin (BSA).

As used herein, the term “ferrofluid magnetic particle” refers to a colloidal magnetic particle that contains iron.

In some embodiments, the magnetic particles are characterized by their sub-micron particle size. In some embodiments, the particles are generally less than about 300 nanometers (nm), 275 nm, 250 nm, 225 nm, 200 nm, 190 nm, 180 nm, 170 nm, 160 nm, 150 nm, 140 nm, 130 nm, 120 nm, 110 nm, or 100 nm in diameter. In some embodiments, the particles are generally at least 10 nm, 20 nm, 30 nm, 40 nm, 50 nm, 60 nm, 70 nm, 80 nm, 90 nm, 100 nm, 110 nm, or 120 nm or more in diameter. In some embodiments, the particles are between about 40 nm to 250 nm, 40 nm to 200 nm, 50 nm to 200 nm, 50 nm to 190nm 50 nm to 180 nm, 50 nm to 170 nm, 60 nm to 200 nm, 70 nm to 200 nm, 80 nm to 200 nm, 90 nm to 200 nm, 90 nm to 175 nm, or 90 nm to 150 nm in diameter.

In some embodiments, the particles have at least 40%, 45%, 50%, 55%, 60%, 65%, 70%, 75%, 80%, 85%, 90%, 95%, or 97% or more magnetic mass. In some embodiments, the particles have between about 40% to 95%, 45% to 95%, 50% to 90%, 55% to 90%, 60% to 90%, or 70% to 90% magnetic mass.

In some embodiments, particles within the range of 90-150 nm and having between 70-90% magnetic mass may be used.

In some embodiments, the particles are characterized by their resistance to gravitational separation from solution. The particles may be resistant to gravitational separation for extended periods of time. The particles may be resistant to gravitational separation for at least 1, 2, 3, 4, 5, 6, 7, 8, 9, 10, 15, 20, 25, 30, 35, 40, 45, 50, 55, 60, 75, 90, 105, or 120 or more minutes. The particles may be resistant to gravitational separation for at least 1, 2, 3, 4, 5, 6, 7, 8, 9, 10, 15, 20, 25, 30, 35, 40, 45, 50, 55, 60, 75, 90, 105, or 120 or more hours. The particles may be resistant to gravitational separation for at least 1, 2, 3, 4, 5, 6, 7, 8, 9, 10, 15, 20, 25, 30, 35, 40, 45, 50, 55, 60, 75, 90, 105, or 120 or more days.

In some embodiments, magnetic particles are composed of a crystalline core of superparamagnetic material surrounded by coating molecules which are bonded, e.g., physically absorbed or covalently attached, to the magnetic core and which confer stabilizing colloidal properties. The coating material may be applied in an amount effective to prevent non-specific interactions between biological macromolecules found in the sample and the magnetic cores. Such biological macromolecules may include sialic acid residues on the surface of non-target cells, lectins, glycoproteins and other membrane components. In addition, the coating material may contain as high a magnetic mass/nanoparticle ratio as possible. The size of the magnetic crystals comprising the core is sufficiently small that they do not contain a complete magnetic domain. The size of the nanoparticles is such that their Brownian energy exceeds their magnetic moment. Consequently, North Pole-South Pole alignment and subsequent mutual attraction/repulsion of these colloidal magnetic particles does not appear to occur even in moderately strong magnetic fields, contributing to their solution stability.

The magnetic particles may be separable in high magnetic gradient external field separators. That characteristic facilitates sample handling and provides economic advantages over the more complicated internal gradient columns loaded with ferromagnetic beads or steel wool.

Magnetic particles may be prepared by modification of base materials as described in EP0842042, which is incorporated by reference in its entirety.

Magnetic particles can be coated with Abs (or more in general any agents) capable of recognizing the differentially expressed proteins corresponding to the top candidates identified in Example 1. In some embodiments, magnetic particles can be coated with an agent that binds to a rare cell marker (e.g., TREML2). Magnetic particles can be coated with any of the antibodies or agents disclosed herein.

Coating of magnetic particles may be performed by any method known in the art. For instance, magnetic particles may be coated with an antibody as described U.S. Pat.No. 6,365,362B1, which is incorporated by reference in it entirety.

FIG. 2 depicts an exemplary ferrofluid magnetic particle structure. A ferrofluid magnetic particle disclosed herein may comprise, consist of, or consist essentially of, the ferrofluid magnetic particle structure shown in FIG. 2. In some embodiments, a ferrofluid magnetic particle disclosed herein has the ferrofluid magnetic particle structure shown in FIG. 2. As shown in FIG. 2, an exemplary ferrofluid magnetic particle structure comprises, consists of, or consists essentially of, an iron atom surrounded by bovine serum albumin (BSA). The BSA is attached to streptavidin (SA), which is attached to biotin (BT). The BT may be attached to another BSA, which is attached to an exogenous aggregation enhancing factor (e.g., desthiobiotin (Dt-BT)). The BT may also be attached to an antibody (Y) that binds to a marker on a rare cell. In some embodiments, the rare cell is a fetal cell. In some embodiments, the marker is TREML2. Alternatively, the marker is EpCAM, CD105, or CD71.

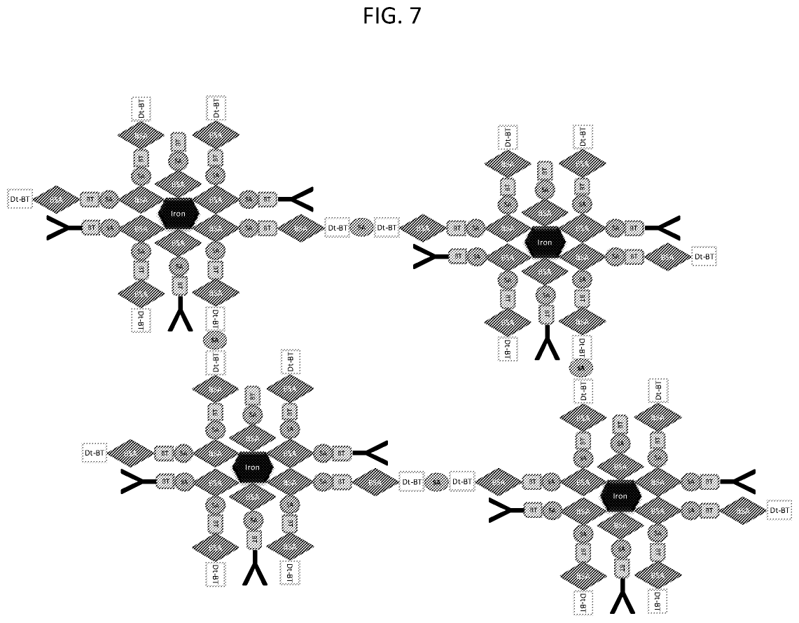

FIG. 7 depicts a schematic of magnetic particle aggregation via controlled aggregation. As shown in FIG. 7, a magnetic particle, such as the ferrofluid magnetic particle of FIG. 2, is coupled to an exogenous aggregation enhancing factor (EAEF, e.g., desthiobiotin (Dt-BT). The addition of a second EAEF (e.g., streptavidin (SA)) that is capable of binding to the first EAEF promotes the aggregation of the antibody-magnetic particle conjugates. In some embodiments, the aggregation of the antibody-magnetic particle conjugates is reversed by the addition of a third EAEF, wherein the third EAEF is capable of binding to the first EAEF or second EAEF. In some embodiments, the third EAEF is identical to the first EAEF. Alternatively, the third EAEF is identical to the second EAEF. In another embodiment, the third EAEF is a binding partner of the first EAEF or second EAEF (e.g. biotin).

Compositions and Kits

Disclosed herein are compositions and kits comprising any of the anti-TREML2 antibodies disclosed herein or an antigen binding fragment thereof. The compositions or kits may further comprise one or more components selected from a magnetic reagent, one or more additional antibodies or antibody conjugates, an aggregation inhibitor, and an aggregation factor.

In some embodiments, the kit comprises (a) anti-TREML2 antibody or an antigen binding fragment thereof; and (b) a magnetic reagent.

In some embodiments, the kit comprises (a) anti-TREML2 antibody or an antigen binding fragment thereof; and (b) a colloidal magnetic particle.

In some embodiments, the kit comprises (a) an anti-TREML2 antibody or antigen binding fragment thereof; and (b) one or more additional antibodies or an antigen binding fragments thereof.

Further disclosed herein is a kit comprising (a) an anti-TREML2 antibody or an antigen binding fragment thereof; and (b) a second antibody or an antigen binding fragment thereof, wherein the second antibody binds to a protein expressed on the surface of a fetal nucleated red blood cell (fnRBC).

Further disclosed herein is a kit comprising (a) an anti-TREML2 antibody or an antigen binding fragment thereof; and (b) a second antibody or an antigen binding fragment thereof, wherein the second antibody is conjugated to a label.

Further disclosed herein is a kit comprising (a) a first anti-TREML2 antibody or an antigen binding fragment thereof, wherein the first anti-TREML2 antibody or antigen binding fragment thereof is conjugated to a magnetic particle; and (b) a second anti-TREML2 antibody or an antigen binding fragment thereof, wherein the second anti-TREML2 antibody is conjugated to a label.

In some embodiments, the composition or kit comprises an anti-TREML2 antibody or antigen binding fragment thereof, wherein the anti-TREML2 antibody or antigen binding fragment thereof comprises (a) a heavy chain variable region (HCVR) comprising, consisting of, or consisting essentially of, (i) a complementarity determining region (CDR) 1 comprising, consisting of, or consisting essentially of, the amino acid sequence of SEQ ID NO: 6; (ii) a CDR2 comprising, consisting of, or consisting essentially of, the amino acid sequence of SEQ ID NO: 7; and (iii) a CDR3 comprising, consisting of, or consisting essentially of, the amino acid sequence of SEQ ID NO: 8; and (b) a light chain variable region (LCVR) comprising, consisting of, or consisting essentially of (i) a CDR1 comprising, consisting of, or consisting essentially of, the amino acid sequence of SEQ ID NO: 9; (ii) a CDR2 comprising, consisting of, or consisting essentially of, the amino acid sequence of SEQ ID NO: 10; and (iii) a CDR3 comprising, consisting of, or consisting essentially of, the amino acid sequence of SEQ ID NO: 11. In some embodiments, any one of SEQ ID Nos: 6-11 independently comprise one or more amino acid substitutions, additions, or deletions.

In some embodiments, the kit comprises (a) an anti-TREML2 antibody or an antigen binding fragment thereof; and (b) a buffer comprising an aggregation inhibitor.

In some embodiments, the kit comprises (a) an anti-TREML2 antibody or an antigen binding fragment thereof; and (b) an exogenous aggregation enhancing factor.

Any of the compositions, kits, or methods disclosed herein may comprise one or more magnetic reagents. The magnetic reagent may comprise one or more magnetic particles. The magnetic reagent may comprise a ferromagnetic particle, supraparamagnetic particle. The magnetic reagent may comprise a ferrofluid reagent.

As used herein, the term “ferromagnetic particle” refers to a particle that is permanently magnetizable.

The magnetic reagent may comprise a supraparamagnetic particle. As used herein, the term “supraparamagnetic particle” may refer to a particle that is a magnetically responsive particle. A supraparamagnetic particle is a particle that demonstrates magnetic behavior only when subjected to a magnetic field. In some embodiments, a colloidal magnetic particle is a supraparamagnetic particle.

In some embodiments, the magnetic reagent comprises a magnetic particle. In some embodiments, the magnetic particle is about 1.5 to about 50 microns, 0.7-1.5 microns, or less than 200 nm in size. In some embodiments, the magnetic particle is less than 200 nm in size. In some embodiments, the magnetic reagent comprises a magnetic particle conjugated to an antibody. In some embodiments, such antibody conjugated to magnetic particles is an antibody that binds to a protein selected from epithelial cell adhesion molecule (EpCAM) and endoglin (CD105). Alternatively, such antibody conjugated to magnetic particles binds to CD147. In a further embodiment, such antibody conjugated to magnetic particles binds to CD45. In another embodiment, such antibody conjugated to magnetic particles binds to a protein expressed on the surface of a fetal cell.

In some embodiments, the magnetic reagent comprises a ferrofluid reagent. As used herein, the term “ferrofluid reagent” refers to liquid suspension comprising magnetic particles. In some embodiments, the ferrofluid reagent comprises a liquid suspension comprising a magnetic particle conjugated to the anti-TREML2 antibody. Alternatively, the ferrofluid reagent comprises a liquid suspension comprising a magnetic particle conjugated to one or more antibodies disclosed herein. In some embodiments, the ferrofluid reagent comprises a liquid suspension comprising a magnetic particle conjugated to an anti-EPCAM antibody. In some embodiments, the ferrofluid reagent comprises a liquid suspension comprising a magnetic particle conjugated to an anti-CD105 antibody. In some embodiments, the ferrofluid reagent comprises a liquid suspension comprising a magnetic particle conjugated to an antibody that binds to a protein expressed on the surface of a fetal cell. In some embodiments, the ferrofluid reagent comprises a liquid suspension comprising a magnetic particle conjugated to an anti-CD147 antibody.

In some embodiments, the kits further comprise one or more staining reagents. In some embodiments, the one or more staining reagents comprise one or more antibody conjugates. In some embodiments, the antibody conjugate of the one or more antibody conjugates is an antibody conjugated to a label. In some embodiments, the antibody binds a protein selected from CD71, glycophorin A (GPA), and CD45.

In some embodiments, any of the antibodies disclosed herein (e.g., anti-TREML2 antibody or one or more additional antibodies) further comprise a label. In some embodiments, the label is conjugated to the antibody. In some embodiments, the label is selected from phycoerythrin (PE), allophycocyanin (APC), horse radish peroxidase (HRP), and biotin.

Any of the kits disclosed herein may comprise one or more antibodies or fragments thereof. The one or more antibodies may bind to a protein expressed on the surface of a fetal cell. Alternatively, or additionally, the one or more antibodies may bind to a protein expressed on the surface of a maternal cell. The one or more antibodies may bind to a protein selected from EpCAM, CD105, CD147, CD15, CD71, GPA, and CD45. The one or more antibodies may bind to a protein selected from CD15, CD71, GPA, and CD45.

Any of the kits disclosed herein may comprise one or more antibodies or fragments thereof, wherein the one or more antibodies bind to a protein expressed on the surface of a fetal nucleated red blood cell (fnRBC) or trophoblasts. The antibody may bind to a protein selected from EpCAM, CD105, CD71 and CD147.

Any of the kits disclosed herein may comprise one or more aggregation inhibitors. The kits disclosed herein may comprise 1, 2, 3, 4, or 5 or more aggregation inhibitors. The aggregation inhibitor may inhibit endogenous ferrofluid aggregation factors. In some embodiments, the aggregation inhibitor is selected from a reducing agent, an immune-complex, a chealting agent and diamino butane. The reducing agent may be mercaptoethane sulfonic acid. The aggregation inhibitor may be a bovine serum albumin (BSA). The chelating agent may be EDTA.

The aggregation inhibitor may comprise an antibody or fragment thereof, wherein the antibody is the same isotype as the anti-TREML2 antibody. The antibody may be a non-specific antibody. In some embodiments, the antibody is a mouse antibody.

Any of the kits disclosed herein may comprise an anti-TREML2 antibody, wherein the anti-TREML2 antibody may be coupled to a ferrofluid. Any of the kits disclosed herein may comprise an anti-TREML2 antibody, wherein the anti-TREML2 antibody is conjugated to a magnetic particle. The magnetic particle may be a colloidal magnetic particle. The magnetic particle may be ferrofluid magnetic particle.

Any of the kits disclosed herein may comprise an exogenous aggregation enhancing factor (EAEF). In some embodiments, the kits disclosed herein comprise 1, 2, 3, 4, or 5 or more EAEFs. In some embodiments, the magnetic particles disclosed herein are coupled to one more EAEFs. In some embodiments, the EAEF comprises one member of a specific binding pair selected from the group comprising biotin-streptavidin, antigen-antibody, receptor-hormone, receptor-ligand, agonist-antagonist, lectin-carbohydrate, Protein A-antibody Fc, and avidin-biotin, biotin analog-avidin, desthiobiotin-streptavidin, desthiobiotin-avidin, iminobiotin-streptavidin, and iminobiotin-avidin.

In some embodiments, the kits disclosed herein comprise two or more EAEFs. In some embodiments, the first EAEF comprises one member of a specific binding pair selected from the group comprising biotin-streptavidin, antigen-antibody, receptor-hormone, receptor-ligand, agonist-antagonist, lectin-carbohydrate, Protein A-antibody Fc, and avidin-biotin, biotin analog-avidin, desthiobiotin-streptavidin, desthiobiotin-avidin, iminobiotin-streptavidin, and iminobiotin-avidin and the second EAEF comprises the other member of the specific binding pair.

In some embodiments, the kits disclosed herein further comprise a third EAEF. In some embodiments, the third EAEF is identical to the first EAEF. Alternatively, the third EAEF is identical to the second EAEF. In another embodiment, the third EAEF is capable of interacting with the first EAEF. In another embodiment, the third EAEF is capable of interacting with the second EAEF. By having a third EAEF that is identical to the first or second EAEF or capable of interacting with the first or second EAEF, the addition of the third EAEF results in a reversing aggregation of the magnetic particles.

In some embodiments, the kits disclosed herein further comprise one or more aggregation inhibiting agents. In some embodiments, the aggregation inhibiting agent is selected from the group consisting of a reducing agent, an immune-complex, a chelating agent, and a diamino butane. In some embodiments, the aggregation inhibiting agent is a chelating agent. In some embodiments, the chelating agent is EDTA. The reducing agent may be mercaptoethane sulfonic acid. The aggregation inhibitor may be a bovine serum albumin (BSA).

### EXAMPLES

### Example 1

### Identification of Novel Markers for Fetal Cells

This examples describes the identification of novel markers for fetal cells.

Preparation of Nucleated Red Blood Cells (nRBCs)

Fetal whole blood (n=5) was obtained by ultrasound-guided procedures from pregnant women (10⁺⁰ 15⁺⁶ gestational weeks) scheduled for surgical termination of pregnancies.

20 mL of peripheral blood was collected from pregnant women at delivery (n=2) or before surgical termination of pregnancy (n=1).

After collection, fetal and maternal blood were diluted with equal volume of phosphate buffered saline (PBS) and slowly layered onto a Percoll gradient. Samples were centrifuged at 1800 rpm for 10 minutes at room temperature. The layer at interphase containing fetal or adult erythroblasts was collected and washed twice with PBS.

For the maternal blood, a depletion step of CD45/CD15 positive cells was performed by labeling cells with anti-CD45 and anti-CD15 microbeads (Miltenyi Biotec), using LD Column (Miltenyi Biotec).

For the maternal blood, a microfluidic device was also used to remove the RBCs contaminants cells and enrich for adult erythroblast.

Enrichment and Cell Sorting by Flow Cytometry

To prepare samples for FACS sorting, the enriched cells from fetal and maternal blood were stained with anti-CD71 antibody (Miltenyi Biotec), anti-GPA antibody (BD Bioscience), anti-CD45 antibody (Miltenyi Biotec), Hoechst (nuclear staining), and Sytox Green dye (live/dead cells) at room temperature for 30 minutes.

FACS Sorting

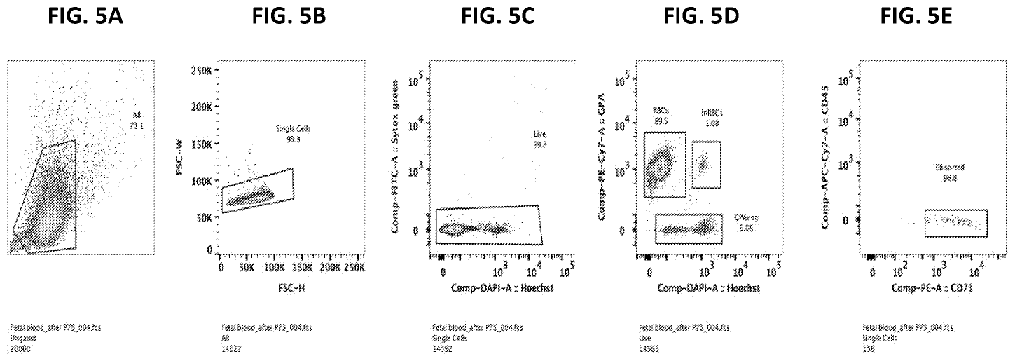

Erythroblasts cells were gated and sorted as shown in FIGS. 5A-5E. FIG. 5B: gate FSC-H/W and exclude doublet cells. FIG. 5C: gate Sytox Green neg live cells. FIG. 5D: gate dp GPA/Hoechst. FIG. 5E: gate CD71pos/CD45neg cells.

For fetal blood samples, less than 200,000 target erythroblasts were sorted.

For maternal blood samples, the number of maternal erythroblasts never exceeded 1,000.

RNA Extraction on Sorted Population

Processed, sorted cells were used for total RNA extraction.

Total RNA was extracted from the sorted cells using Picopure RNA Isolation Kit (Applied Biosystems) and quantified by Quant-iT RiboGreen RNA Assay Kit (Thermo Fisher) and analyzed for quality control using RNA 6000 Pico Kit on Agilent 2100 Bioanalyzer.

RNAseq Preparation and Library Preparation

The cDNA library was prepared from Illumina Sequencing (Appendix A; RNA-Seq protocol). Sequencing was performed on HiSeq 2000 at 20 million reads/sample.

Sequencing

Reads resulting from next generation (Illumina) sequencing were first checked for quality with standard procedures using FastQC protocol, then mapped to the reference genome and subsequently quantified using STAR software version 2.5. The generated read-counts matrices (i.e., the tables of read-counts for all detected feature—coding or on coding RNAs—in each sample) were reduced in dimension with a data reduction step, keeping only genes with at least a single count in a single sample.

Data Analysis with Bioinformatics Tools

The resulting data were further processed with DESeq2 R/Biocondutor package for differential expression to find the genes whose expression is significantly higher or lower between the two compared sample types.

A default DESeq2 differential expression analysis was performed consisting of the following steps: for each sample estimation of size factors using the “median ratio method” (Anders and Huber, 2010), for each gene, an estimate of the dispersion is found with a fitting procedure optimizing the dispersion for Negative Binomial distributed data, finally the obtained size factors and dispersions estimates were used to test for significance of coefficients of the fitted distribution.

The result table from the DESeq2 analysis was finally extracted obtaining base means across samples, log2 fold changes, standard errors, test statistics, p-values and adjusted p-values for 20205 features (genes) with nonzero total read count. Differentially expressed genes were filtered according to an adjusted p-value (Benjamini-Hockberg/FDR method) cutoff of 0.01 and a 2 fold-change expression cut-off. With these parameters 3233 genes were selected as differentially expressed, most of them (2961) upregulated (independently from fold change cut-off) in fetal blood.

The resulting gene table was decorated with annotations and functional descriptions extracted from the Ensembl database. The genes associated to “Plasma Membrane” annotation according to the Gene Ontology term GO:0005886 were flagged and additionally tagged as “Transmembrane” getting the data from Uniprot database. Among those, 366 plasma membrane genes were differentially expressed, most of them (336) upregulated (independently from fold change cut-off) in the fetal cells.

In parallel to the differentially expression analysis, read counts were normalized and transformed using DESeq2 in order to operate a selection on more stringent expression criteria: the first selection was performed starting from the list of genes exclusively expressed in fetal samples, i.e., those with zero reads in all three maternal samples (12187 genes, list “ALL DATA”), according the following criteria: 1) selection of genes detected (i.e. expressed) in all fetal samples; 2) selection of genes with an expression mean/sd ratio higher than 1; 3) selection of genes both associated to the Plasma Membrane GO tag and tagged as “Transmembrane Predicted” by the Uniprot database. See Selection Scheme below. The resulting 77 genes were then ranked according to decreasing fetal mean expression (list “RANKED”). A final selection by manual curation was performed taking into consideration known biological function, stability of expression level across samples and crude estimation of absolute expression level (reads compared to gene length), antibody availability and other biological consideration (16 genes, list “Selected”).

Selection Scheme

The following is the selection scheme used for identifying potential novel markers for fetal cells:

1) genes NOT expressed in any maternal sample: 12187

2) genes that are exclusively expressed in ALL fetal samples: 2079

2.1) genes annotated as associated to GO Plasma Membrane term: 213

2.2) genes predicted by UniProt as Transmembrane: 305

3) genes both associated to GO Plasma Membrane and predicted as Transmembrane: 89

4) genes whose mean/sd ratio is higher than 1:77

Ranking of Genes Differentially Expressed From the Gene List

A further final, manually curated, selection and ranking procedure was performed taking into consideration transcript length, number of reads and other biologically relevant criteria, and the resulting top candidates were the following:

TABLE 1
Top Candidates for Novel Markers for Fetal Cells

| | | | |
|---|---|---|---|
| TREML2 | STIM1 | TNFSF18 | SCARF |
| PTPRN | SELPLG | LRP11 | ESYT1 |
| BMPR2 | GPR160 | ABCA1 | SCARB1 |
| TNFRSF19 | KLRD1 | GPR176 | LTB4R |

Identification of Antibodies Specific for Selected Target Molecules

Testing Ab Candidates for Erythroblasts by FACS Analysis

In order to determine if the differential expression in RNA levels are reflected in the levels of the respective proteins, immunostaining with the commercial available antibodies (n=13) for flow cytometry and DEPArray analysis was performed.

As negative controls, isotype matched Abs, which were conjugated with the same fluorochromes as the commercial antibodies, were used in the same concentrations.

All 13 antibodies shown in Table 2 were tested on frozen fetal blood first.

Only the antibodies positively expressed on fetal erythroblasts were tested on frozen maternal blood samples.

Besides the specific antibodies to test or isotype controls, antibodies used for staining includes CD71 Ab (Miltenyi Biotec), GPA Ab (BD Bioscience), CD45 Ab (Miltenyi Biotec), Hoechst, for the Erythroblast identification. In brief, cells (2.5−5×10⁵) were incubated with the Abs in the presence of FcR Blocking reagents (Miltenyi Biotec) at room temperature for 30 minutes. After washing out unbound Abs, cell pellets were resuspended with AutoMACS Running buffer (Miltenyi) with Sytox Green dye.

TABLE 2
FACS Analysis results

| | | Erythroblast | | Reticulocytes | | RBCs | | CD45 pos | | | |
|---|---|---|---|---|---|---|---|---|---|---|---|
| | Antibody | Fetal | Maternal | Fetal | Maternal | Fetal | Maternal | Fetal | Maternal | Isotype | Conjugated |
| 1 | TLT2 (MIH61) | Low | No | Low | No | No | No | No | No | Mouse IgG1 | PE |
| 6 | TNFRSF19 | High | High | High | High | High | High | No | No | Mouse IgG1 | PE |
| 11 | STIM1 | Int | Int | Int | Int | Low | Low | High | NA | Rabbit Polyclonal | Unconjugated |
| 2 | TNFSF18 | No | NA | No | NA | No | NA | No | NA | Mouse IgG1 | PE |
| 2 | TNFSF18 | No | NA | No | NA | No | NA | No | NA | Human IgG1 | PE |
| 3 | PTPRN | No | NA | No | NA | No | NA | No | NA | Rabbit Polyclonal | Unconjugated |
| 4 | LRP-11 | No | NA | No | NA | No | NA | No | NA | Rabbit Polyclonal | FITC |
| 4 | LRP-11 | No | NA | No | NA | No | NA | No | NA | Sheep Polyclonal | Unconjugated |
| 5 | BMPR2 | No | NA | No | NA | No | NA | No | NA | Rabbit Polyclonal | Unconjugated |
| 5 | BMPR2 | No | NA | No | NA | No | NA | No | NA | Mouse IgG1 | Unconjugated |
| 7 | GPR176 | No | NA | No | NA | No | NA | No | NA | Rabbit recomb. | Unconjugated |
| 8 | SCARF1 | No | NA | No | NA | No | NA | No | NA | Mouse IgG2 | AF647 |
| 9 | SELPLG (KLP-1) | No | NA | No | NA | No | NA | High | NA | Mouse IgG1 | PE |
| 9 | SELPLG (HECA452) | No | NA | No | NA | No | NA | Int | NA | Rat IgM | PE |
| 10 | GPR160 | No | NA | No | NA | No | NA | Int | NA | Rabbit Polyclonal | Unconjugated |
| 12 | LTB4R1 (203/14F11) | No | NA | No | NA | No | NA | Int | NA | Mouse IgG1 | PE |
| 12 | LTB4R1 (202/7B1) | No | NA | No | NA | No | NA | No | NA | Mouse IgG2 | PE |
| 13 | KLRD1 (DX22) | No | NA | No | NA | No | NA | High | NA | Mouse IgG1 | PE |
| 13 | KLRD1 (REA113) | No | NA | No | NA | No | NA | High | NA | Human IgG1 | PE |

Ab 1 TLT2 was Ised for TREML2 (This Latter Being Also Referred to as TLS-1 Herein)

### Example 2

### Ferrofluid Technology for Cell Capture and Selection

In this example, selected antibodies, exclusively expressed of fetal cells, were used for ferrofluid conjugation. TREML2-FF (also referred as TLS1-FF) refers to an antibody capable of binding the protein expressed by TLS1 gene that is conjugated to a ferrofluid

The size of the FF-Ab was checked by using the NanoBrook Zeta Plus particle size analyzer, and the concentration with the Spectrophotometer.

| Sample ID | Eff. Diam (nm) | Polydispersity | Baseline Index |
|---|---|---|---|
| TREML2-FF | 188.41 | 0.206 | 7.9 |
| TREML2--FF | 189.36 | 0.217 | 8.6 |
| TREML2-FF | 188.17 | 0.203 | 8.4 |
| TREML2-FF | 190.88 | 0.213 | 8.3 |
| TREML2-FF | 191.03 | 0.216 | 7.8 |
| Mean: | 189.57 | 0.211 | 8.2 |
| Std Err: | 0.6 | 0.003 | 0.2 |
| Std Dev | 1.34 | 0.006 | 0.4 |

Controlled Enrichment

The blood sample is preincubated with a buffer containing one or more inhibitors to inhibit endogenous ferrofluid aggregation factors (as described in EP1311820, which is incorporated by reference in its entirety) before ferrofluid is added to the blood. One of the inhibitors can be a reducing agent, such as mercaptoethane sulfonic acid at 100 mM, which can disable IgM-induced aggregation without affecting the ligands used for labeling cells. The reducing agent can be added as a single reagent to the blood. The second inhibitor can be bovine serum albumin, which can be included in the buffer at 10 mg/ml, and will neutralize any HABAA. The third inhibitor can be nonspecific mouse antibody, in particular, the appropriate isotype which matches the antibody on the ferrofluid. This can be included in the buffer at a concentration of 0.5-5 mg/ml to neutralize even the most severe HAMA. The fourth inhibitor can be Streptavidin to be included in the buffer, if necessary, to neutralize any anti-streptavidin antibody present in plasma. The pre-treatment of blood with the above buffer and reducing agent can be from 15-30 minutes to neutralize all endogenous aggregation factors. After all endogenous aggregation factors are neutralized, an exogenous ferrofluid aggregation factor is added to the sample, followed by ferrofluid. The ferrofluid is coupled to an antibody specific for targets, as well as to another ligand specific for the exogenous aggregation factor. After optimum labeling of target cells with ferrofluid and induced aggregation of ferrofluid with exogenous aggregation factor, the sample is subjected to magnetic separation to enrich targets.

The sample is placed in a magnetic separator (Immunicon catalog No. QS-012) for 10 minutes. The sample is taken out of the magnet and the sample mixed by vortexing, and placed back in the magnetic separator for 10 minutes for collection of magnetically labeled cells. The uncollected sample is aspirated and the magnetically collected cells were resuspended in 0.75 ml of wash-dilution buffer and re-separated in a magnetic separator for 10 minutes. The uncollected sample was discarded and the collected cells were resuspended after removal of the tube from the magnetic separator. After removing all non-targets, magnetically-labeled targets and free ferrofluid are resuspended in a buffer. In cases, exogenous mediated-ferrofluid aggregation should be reversed. This can be achieved by resuspending the final sample in a buffer containing a disaggregation factor which binds to exogenous aggregation factor. The disaggregation factor disaggregates all ferrofluid aggregates, leaving cells easy for further analysis.

### Example 3

### Detection and Analysis of Fetal Cells

This example describes the isolation and analysis of single fetal cells. DEPArray may be performed as described in EP2152859, which is incorporated by reference in its entirety.

Pregnant Women and Healthy Volunteers

Peripheral blood samples were drawn by venipuncture into 10 mL CellSave Preservative Tubes (Menarini Silicon Biosystems, Huntingdon Valley Pa., USA) from 14 pregnant women within the 12 to 17+2 gestational weeks. For spiking experiments, peripheral blood were drawn from healthy donors. All the donors provided written informed consent and the study protocol was approved by the medical ethical committee of the San Gerardo Hospital, Monza (Italy). All the samples were processed after 1-4 days.

Antibodies directed against the epithelial cell adhesion antigen (EpCAM), vascular endothelial marker (CD105) and/or TREML 2 coupled to ferrofluids were used to enrich fetal trophoblast from the contaminants cells. The enriched cells were labeled with anti-TREML2 monoclonal antibody (mAb) labelled with phycoerythrin (PE). The enriched cells were also fluorescently labeled with anti-cytokeratin mAb C11 labelled with allophycocyanin (APC), anti-HLA-G mAb labelled with APC, and anti-CD45 mAb labeled with fluorescein isothiocyanate (FITC) for recognizing leukocytes.

These enrichment procedures of the target cells (e.g.; trophoblast cells) are necessary since it is known that the frequency of said cells in the maternal blood is extremely low, only 1-10 cells within 1 ml of total blood (which contains more than one billion cells).

CVS and Cord Blood for Spiking.

Whole blood from healthy volunteers were spiked with fetal trophoblast cells derived from Chorionic Villous Sampling (CVS) or fetal erythroblast cells derived from Cord blood.

The CVS culture was chosen for its CD105/EpCAM expression. The cells were grown at 37° C. and 5% CO₂ in RPMI 1640 (Gibco) supplemented with 10% fetal bovine serum (Gibco), 1% penicillin-streptomycin (Gibco) and L-Glutamine (Gibco). Before the spiking, the cells were detached from the flask, resuspended into 10 ml of PBS (Gibco) and placed into CellSave Preservative Tube for at least 1 day.

Cord blood samples obtained by San Gerardo Hospital were collected into CellSave Preservative tube.

The spiking experiments were carried out to demonstrate the specificity of the selection procedure when using ferrofluid conjugated-antibodies to capture fetal cells.

Fetal Blood and Bone Marrow Samples

Fetal whole blood (n=3) was obtained by ultrasound-guided procedures from pregnant women (10⁺⁰ 15⁺⁶ gestational weeks) scheduled for surgical termination of pregnancies.

All the donors provided written informed consent and the study protocol was approved by the medical ethical committee of the KK Women's and Children's Hospital, Singapore.

After collection, fetal blood was diluted with equal volume of PBS and slowly layered onto Percoll gradient. Sample was centrifuge at 1800 rpm for 10min at room temperature. The layer at interphase containing fetal erythroblasts was collected and washed twice with PBS.

Cryopreserved Bone Marrow mononuclear cells containing adult erythroblast, were purchased (Lonza, Cat. 2M-125C). Cells were thawed and DNasel treated and washed as manufacturer's instruction. Cells were then rested in RPMI medium supplied with 10% FBS, Penicillin/Streptomycin, L-Glutamine at 37 degrees for 1 hour and used as negative control (3 different donors were tested).

Four clones of commercially available TREML2 antibodies were tested in samples by using flow cytometry.

Isotype matched Abs which were conjugated with the same fluorochromes as the commercial TREML2 Ab were used in the same concentrations. Besides TREML2 Ab or isotype controls, Ab used for staining includes CD71 Ab (Miltenyi Biotec), GPA Ab (BD Bioscience), CD45 Ab (Miltenyi Biotec), Hoechst. In brief, cells (2.5—5×10⁵) were incubated with the Abs in the presence of FcR Blocking reagents (Miltenyi Biotec) at room temperature for 30 min. After washing out unbound Abs, cell pellets were resuspended with Running buffer with Sytox Green dye to gate on live cells for FACS analysis.

Preparation of Desthiobiotin Ferrofluid Antibodies for Controlled Aggregation

In some embodiments, the ferrofluids for use in carrying out this invention are particles that behave as colloids. Such particles are characterized by their sub-micron particle size, which is generally less than 200 nanometers (nm), and their resistance to gravitational separation from solution for extended periods of time. Particles within the range of 90-150 nm and having between 70-90% magnetic mass are used. Suitable magnetic particles are composed of a crystalline core of superparamagnetic material surrounded by coating molecules which are bonded, e.g., physically absorbed or covalently attached, to the magnetic core and which confer stabilizing colloidal properties. The coating material should preferably be applied in an amount effective to prevent non-specific interactions between biological macromolecules found in the sample and the magnetic cores. Such biological macromolecules may include sialic acid residues on the surface of non-target cells, lectins, glycoproteins and other membrane components. In addition, the coating material should contain as high a magnetic mass/nanoparticle ratio as possible. The size of the magnetic crystals comprising the core is sufficiently small that they do not contain a complete magnetic domain. The size of the nanoparticles is such that their Brownian energy exceeds their magnetic moment. Consequently, North Pole-South Pole alignment and subsequent mutual attraction/repulsion of these colloidal magnetic particles does not appear to occur even in moderately strong magnetic fields, contributing to their solution stability. Finally, the magnetic particles should be separable in high magnetic gradient external field separators. That characteristic facilitates sample handling and provides economic advantages over the more complicated internal gradient columns loaded with ferromagnetic beads or steel wool. Magnetic particles having the above-described properties can be prepared by modification of base materials described in EP0842042. In a preferred embodiment of the invention, magnetic particles coated with anti-CD105 antibody are prepared as described U.S. Pat. No. 6,365,362B1, which is incorporated by reference in its entirety.

Recombinant human antibody to the CD105 antigen was obtained from hybridoma number 166707 (R&D Systems) and conjugated to base material by standard coupling chemistry, as described in U.S. patent application Ser. No. 09/248,388. CD105 Ab ferrofluid was then resuspended in 20 mM HEPES, pH 7.5 for conjugation to desthiobiotin using N-hydroxysuccinimide-DL-desthiobiotin (NHS-desthiobiotin) (Sigma, Cat. # H-2134). A stock solution of NHS desthiobiotin was made in DMSO at 1 mg/ml. NHS-desthiobiotin (5 mg) was added to 1 mg of CD105 Ab ferrofluid and incubated at room temperature for 2 hours. Unreacted NHS-Desthiobiotin was removed by washing three times with 20 mM HEPES, oH 7.5 containing 1 mg/ml BSA, 0.05% Proclin 300 using a high gradient magnet. After the final wash, desthiobiotin/CD105 Ab ferrofluid was resuspended in Water/BSA/Proclin 300 and filtered through 0.2 um syringe filter. Iron Concentration of CD105 Ab ferrofluid was determined using a spectrophotometric assay and adjusted to 0.22 mg/ml. A particle sizer determination was performed by using the particle sizer analyzer NanoBrook 90Plus (Brookhaven Instruments Corporation).

Anti-CD71, anti-TREML2 and anti-EpCAM antibodies were conjugated to the ferrofluid by using the same method.

Processing Blood

Aliquots of 7.5 ml of blood were diluted with 6.5 ml of Dilution Buffer (Menarini Silicon Biosystems).

The blood sample (aliquots of 7.5 ml) is preincubated with 6.5 ml of Dilution Buffer (Menarini Silicon Biosystems) containing one or more inhibitors to inhibit endogenous ferrofluid aggregation factors (as described in EP1311820, which is incorporated by reference in its entirety) before ferrofluid is added to the blood. One of the inhibitors can be a reducing agent, such as mercaptoethane sulfonic acid at 100mM, which can disable IgM-induced aggregation without affecting the ligands used for labeling cells. The reducing agent can be added as a single reagent to the blood. The second inhibitor can be bovine serum albumin, which can be included in the buffer at 10 mg/ml, and will neutralize any HABAA. The third inhibitor can be nonspecific mouse antibody, in particular, the appropriate isotype which matches the antibody on the ferrofluid. This can be included in the buffer at a concentration of 0.5-5 mg/ml to neutralize even the most severe HAMA. The fourth inhibitor can be Streptavidin to be included in the buffer, if necessary, to neutralize any anti-streptavidin antibody present in plasma. The pre-treatment of blood with the above buffer and reducing agent can be from 15-30 minutes to neutralize all endogenous aggregation factors. During this incubation time, the diluted blood was centrifuged at 800 g for 10 min without brake at room temperature for plasma removal.

After all endogenous aggregation factors are neutralized, an exogenous ferrofluid aggregation factor (streptavidin) is added to the sample, followed by ferrofluid. The ferrofluid is coupled to an antibody specific for targets, as well as to another ligand specific for the exogenous aggregation factor such as, for example, desthiobiotin (binding pair desthiobiotin-streptavidin).

Anti-CD105 ferrofluid, anti-EpCAM ferrofluid and/or anti-TREML2 ferrofluid were used for fetal trophoblast enrichment. Anti-CD71 ferrofluid and/or anti-TREML2 ferrofluid were used for fetal erythroblast enrichment. After optimum labeling of target cells with ferrofluid and induced aggregation of ferrofluid with exogenous aggregation factor, the sample is subjected to magnetic separation to enrich targets.

The sample was placed in a magnetic separator (Immunicon catalog No. QS-012) for 10 minutes. The sample was taken out of the magnet and the sample was mixed by vortexing, and placed back in the magnetic separator for other 10 minutes. The sample was taken out of the magnet and mixed once again, and placed back in the magnetic separator for other 20 minutes, for collection of magnetically labeled cells. The uncollected sample was aspirated and the magnetically collected cells were resuspended in 3 ml of wash-dilution buffer and re-separated in a magnetic separator for 10 minutes. The uncollected sample was discarded and the collected cells were resuspended after removal of the tube from the magnetic separator. After removing all non-targets, magnetically-labeled targets and free ferrofluids are resuspended in a buffer. In some cases, exogenous mediated-ferrofluid aggregation is reversed. Reversal of aggregation can be achieved by resuspending the final sample in a buffer containing a disaggregation factor which binds to the exogenous aggregation factor (in case of the binding pair desthiobiotin-streptavidin the exogenous agent that reverts aggregation may be biotin). Without wishing to be bound by theory, the disaggregation factor disaggregates all ferrofluid aggregates, leaving cells for further analysis.

For trophoblasts, the enriched cells were fluorescently labeled with anti-TREML2 monoclonal antibody (mAb) labelled with phycoerythrin (PE). For trophoblasts, the enriched cells were also fluorescently labeled with the nucleic acid dye (Hoechst 33342) for DNA staining, anti-cytokeratin mAb C11 labelled with allophycocyanin (APC), anti-HLA-G mAb labelled with APC, and/or anti-CD45 mAb labeled with Fluorescein isothiocyanate (FITC) for recognizing leukocytes.

For erythroblasts, the enriched cells were fluorescently labeled with anti-TREML2 monoclonal antibody (mAb) labelled with phycoerythrin (PE). For erythroblasts, the enriched cells were also fluorescently labeled with the nucleic acid dye (Hoechst 33342) for DNA staining, anti-CD71 monoclonal antibody (mAb) labelled with phycoerythrin (PE) and/or anti-CD45 mAb labeled with Fluorescein isothiocyanate (FITC).

Stained cells were fixed with 2% Paraformaldehyde (PFA) for 20 minutes at room temperature, then washed and resuspended in a proper buffer and volume for DEPArray™ NxT System (Menarini Silicon Biosystems), or FACS analysis.

DEPArray Analysis

The DEPArray™ NxT is a semiconductor based technology for precise isolation of pure single cells. It's composed of the DEPArray™ Control Unit and the single-use Cartridge which combines state-of-the-art microfluidic and silicon biochip technology to gently manipulate each single target cell in an enriched sample.

The phenomenon that allows cells to be manipulated inside the chip is called “dielectrophoresis” and is based on the capacity to polarize a particle inside a liquid suspension medium through the action of electric fields. Polarization creates a field of force that may be used to trap each individual particle in a row of potential wells, thus allowing the position of the particles to be controlled. Each potential well can be controlled by modifying the programming of the chip in order to move one or more particles from their initial position to their final destination for recovery.

DEPArray™ allows to select and isolate rare cells with an extremely high resolution (down to a single cell) and extremely high purity; the cells are selected through the multiparametric analysis of fluorescence signals and morphological characteristics obtained by processing bright-field or fluorescence images.

This technology has already been used for the isolation and selection of single circulating tumor cells in blood of patients with tumor (as described in EP1311820, which is incorporated by reference in its entirey).

Whole blood samples from healthy volunteers were spiked with Chorionic Villous Culture containing fetal trophoblast cell with trisomy 21. Samples were enriched and stained as previously described.

Trophoblast cells were analyzed on the DEPArray™ NxT System. Trophoblast cells showed positive staining for TREML2. Furthermore, cells showing positive staining for pan-cytokeratin (CK) and undetectable CD45 labelling and positive nuclear staining were classified as fetal trophoblast cells, and isolated as single cells.

Whole blood samples from healthy volunteers were spiked with Cord Blood containing fetal erythroblast cell pre-labeled with Draq5 Nuclear Dye. Samples were enriched with CD71-Ab ferrofluid and TREML2-Ab ferrofluid and stained as previously described. Erythroblast enriched cells were analyzed on the DEPArray™ NxT System. Cells showing positive staining for CD71, undetectable CD45 labelling and positive nuclear staining for Hoechst/Draq5 were classified as fetal erythroblast cells.

Demonstration of Fetal Cell Origin by Short Tandem Repeat (STR) Analysis

Isolated cells were lysed using the DEPArray™ LysePrep Kit (MSB, Italy) according to the manufacturer's instructions.

DNA from single cells were PCR amplified using PowerPlex Fusion 6c human DNA amplification kit (Promega TMD045), which consists of a multiplex primer set targeted to 27 loci across the human genome.

Genomic DNA was also isolated from 200 ul of maternal whole blood using the QlAgen DSP Blood Mini Kit (QIAgen) to serve as controls. When available, fetal genomic DNA obtained from either direct or cultured CVS tissue or amniotic fluid was also analyzed.

STR was carried out according to the manufacturer's recommendations, and the Fragment analyses were carried out using a ThermoFisher Scientific 3500 Genetic Analyzer (POP-4 and 36 cm capillary array); subsequent software analyses were performed using GeneMapper® ID-X v1.4. The allele patterns of the isolated single cells were then compared to the fetal and parental genomic DNA patterns to assess for allelic dropout and expected inheritance patterns.

POC: Clinical Study on 20 Pregnant Women in the First Trimester of Pregnancy

20 ml of Peripheral blood samples were drawn by venipuncture into 10 mL CellSave Preservative Tubes (Menarini Silicon Biosystems, Huntingdon Valley Pa., USA) from 14 pregnant women within the 12 to 17+2 gestational weeks. All the samples were processed after 1-4 days. Fetal trophoblasts were successful isolated from 14 pregnant (Tab. X). A mean of 1.4 fetal trophoblast was isolated from the 14 positive pregnant women.

Copy Number Variant Analysis (CNV)

Whole blood samples from healthy volunteers were spiked with Chorionic Villous culture containing fetal trophoblast cell. Samples were enriched and stained as previously described.

Whole genome amplification (Ampli1 WGA, Menarini Silicon Biosystems) was performed on single fetal trophoblast recovered from DEPArray™ NxT.

5 μl of Ampli™ WGA product were purified with 1.8X SPRIselect Beads (Beckman Coulter) according to manufacturer instructions and eluted in 12.5 μl of TE buffer for library preparation using the Ampl1™ Low pass kit (Menarini Silicon Biosystems).

FASTQ files from 13 Ampli™ LowPass libraries were aligned on hg19 reference genome using BWA. Copy-number profiles were computed using Control-FREEC, without control samples and with GC-normalization. Copy-number plots were obtained using custom python scripts.

Results

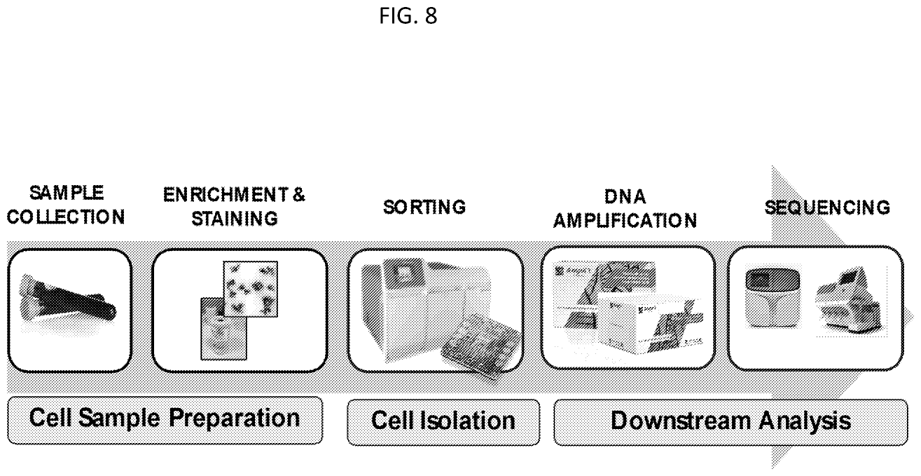

FIG. 8 shows a schematic of the workflow for fetal cell enrichment. As shown in FIG. 8, the workflow consists of 3 separate steps: 1. sample collection and capture of the target cells using ferro-fluid conjugated antibodies that specifically select target cells. 2. Target cells are labeled with selected antibodies and loaded in DEPArray cardridge for screening and selection. Selected single cells are then sorted using the DEPArray instrument. 3. Sorted single cells are analyzed by STR (Short Tandem Repeat) technologies to demonstrate their fetal cell origin.

In this example, fetal cells were enriched and stained from whole blood of pregnant women. Pure single cells are isolated by using of DEPArray™ for whole genome amplification and genome analysis.

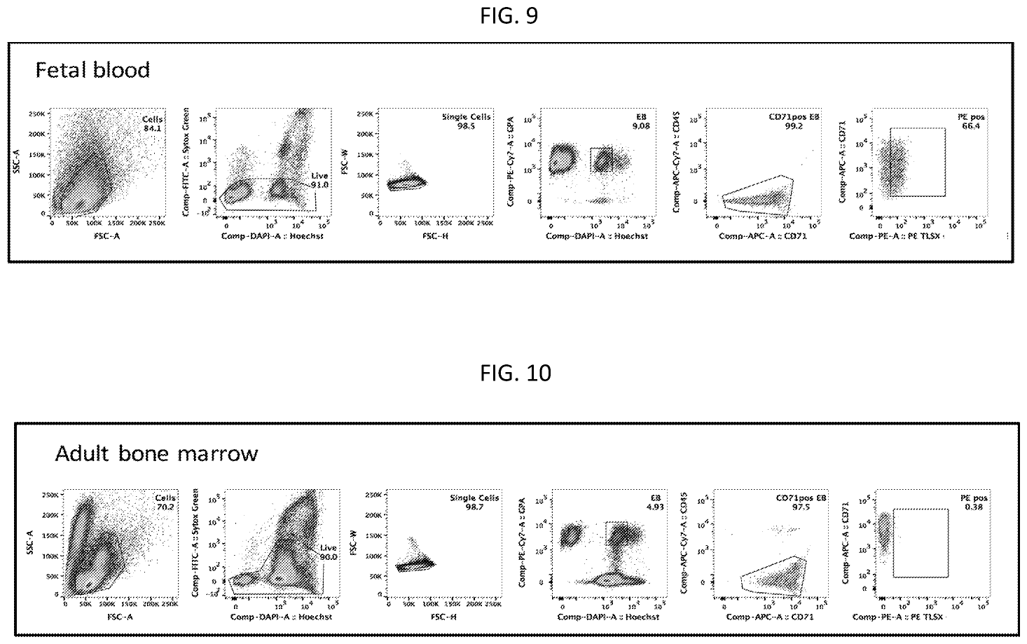

FIGS. 9-10 demonstrate the specificity of the novel TREML2 antibody, as demonstrated by flow cytometry analysis of TREML2 (i.e., TLS) expression on erythroblast isolated from fetal blood (FB) (FIG. 9) and bone marrow samples (BM) (FIG. 10). As shown in FIGS. 9-10, erythroblasts isolated from fetal blood or bone marrow samples were gated by (1) FSC-A/SSC-A to gate major cell population, (2) gate Sytox Green negative live cells, (3) FSC-H/W to exclude doublet cells, (4) gate double positive GPA/Hoechst, (5) gate CD71pos/CD45neg, and (6) gate TLS and overlay with the Isotype control to determine the % of TREML2 positive cells.

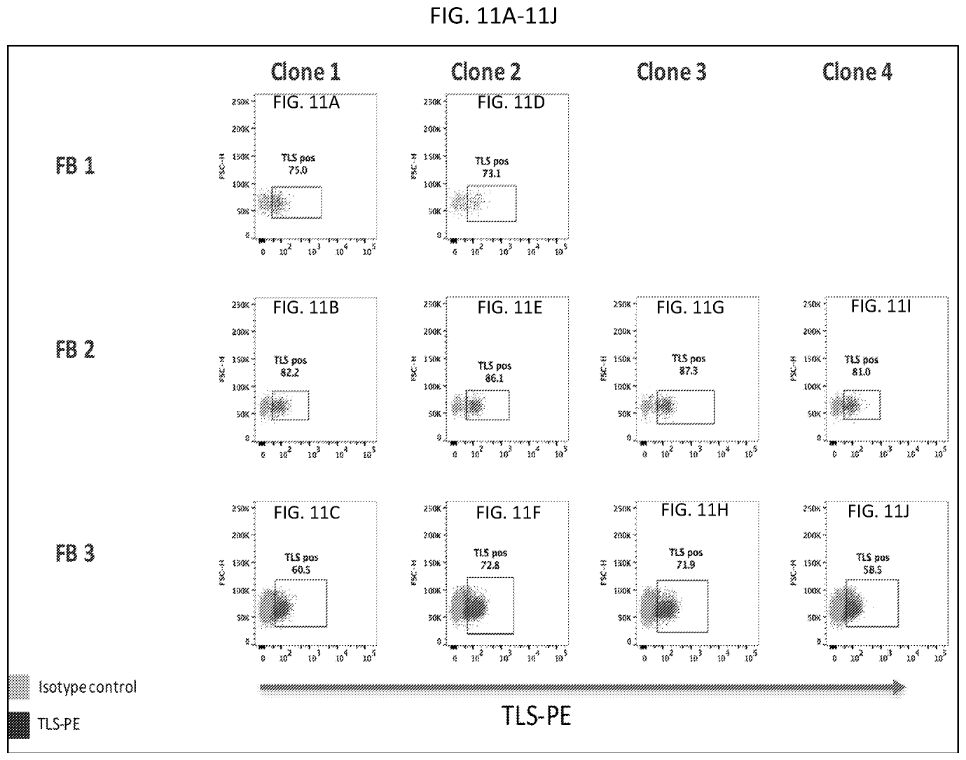

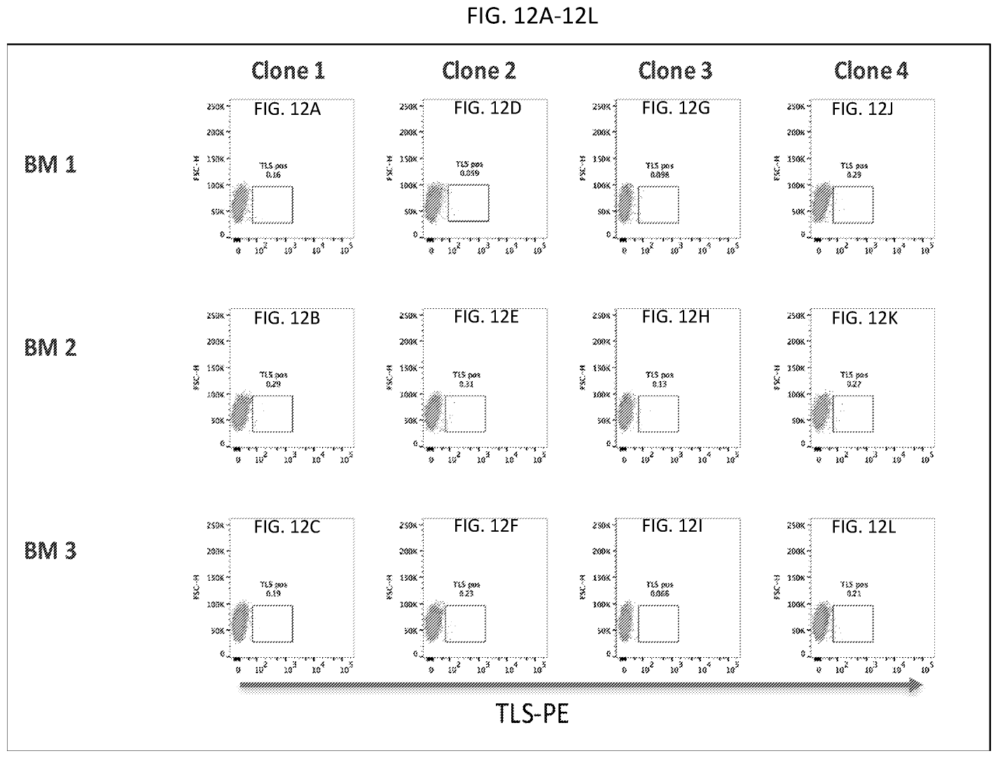

FIGS. 11A-11J shows TLS expression on erythroblast isolated from various fetal blood (FB) samples from various clones. FIGS. 12A-12L shows TLS expression on erythroblast isolated from various bone marrow (BM) samples from various clones. As shown in FIGS. 11A-11J, fetal erythroblast from fetal blood shown positive staining for TREML2 antibody, while no expression is detectable for adult erythroblast isolated from bone marrow (FIGS. 12A-12L).

TABLE 3
Size measurement Ab ferrofluid. The mean
Diameter (nm) for CD105-FF was 128.70 nm

| | | | [symbol] | | | [symbol] | [symbol] | [symbol] |
|---|---|---|---|---|---|---|---|---|
| Type | [symbol] | Sample ID | ( [symbol] ) | [symbol] | [symbol] | ( [symbol] ) | ( [symbol] ) | ( [symbol] ) |
| [symbol] LS | [symbol] | [symbol] - 1 | [symbol] | [symbol] | [symbol] | [symbol] | [symbol] | [symbol] |
| [symbol] LS | [symbol] | [symbol] - 2 | [symbol] | [symbol] | [symbol] | [symbol] | [symbol] | [symbol] |
| [symbol] LS | [symbol] | [symbol] - 3 | [symbol] | [symbol] | [symbol] | [symbol] | [symbol] | [symbol] |
| | | Mean: | [symbol] | [symbol] | [symbol] | [symbol] | [symbol] | [symbol] |
| | | Std Err: | [symbol] | [symbol] | [symbol] | [symbol] | [symbol] | [symbol] |
| | | Std Dev | [symbol] | [symbol] | [symbol] | [symbol] | [symbol] | [symbol] |

[symbol]

indicates data missing or illegible when filed

TABLE 4
Size measurement Ab ferrofluid. The mean Diameter
(nm) for TREML-2-FF was 189.57. Both the size
are within the range of colloidal particles.

| Sample ID | Eff. Diam (nm) | Polydispersity | Baseline Index |
|---|---|---|---|
| TLS-FF | 188.41 | 0.206 | 7.9 |
| TLS-FF | 189.36 | 0.217 | 8.6 |
| TLS-FF | 188.17 | 0.203 | 8.4 |
| TLS-FF | 190.88 | 0.213 | 8.3 |
| TLS-FF | 191.03 | 0.216 | 7.8 |
| Mean: | 189.57 | 0.211 | 8.2 |
| Std Err: | 0.6 | 0.003 | 0.2 |
| Std Dev | 1.34 | 0.006 | 0.4 |

Trophoblast derived from CVS cultures were used to demonstrate specificity of CD105-FF and EpCAM-FF capture and enrichment.

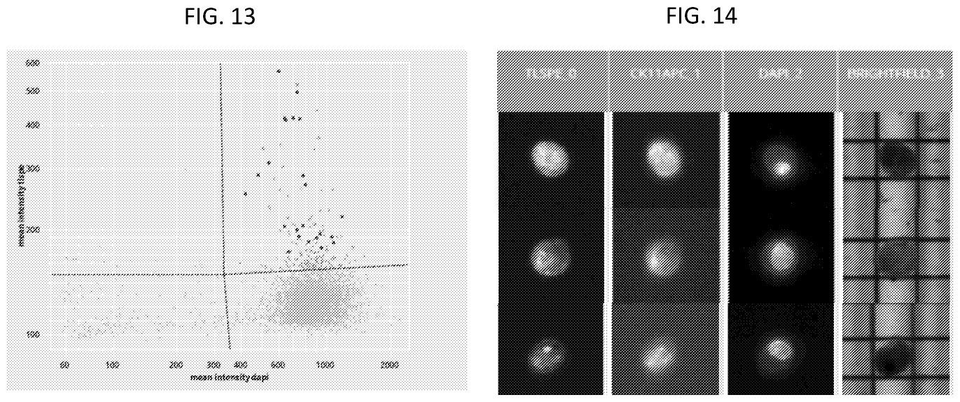

FIG. 13 shows a scatter plot analysis of TREML-2 positive trophoblast cells identified by DEPArray™ after spiking and enrichment with CD105-FF and EpCAM-FF. FIG. 14 shows a CellBrowser® Image gallery: Trophoblast cells shown positive staining from TREML-2-PE antibody, CK-APC and nuclear staining.

Erythroblasts derived from cord blood were used to demonstrate specificity of CD71 or TREML-2 capture and enrichment.

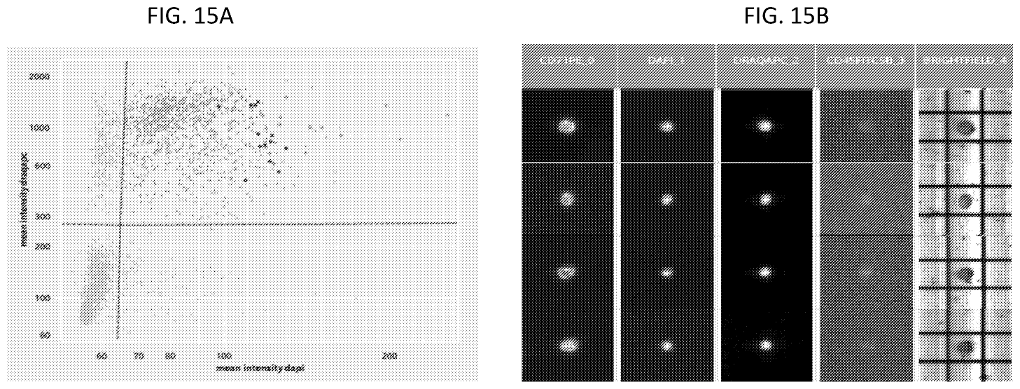

FIG. 15A shows a scatter plot analysis of Draq5/Hoechst positive erythroblasts spiked in Healthy donor blood and enriched with CD71-FF. FIG. 15B shows a CellBrowser® Image gallery: erythroblast cells shown positive staining from CD71-PE antibody, Draq5 and Hoechst nuclear staining, and negative staining for CD45-FITC antibody.

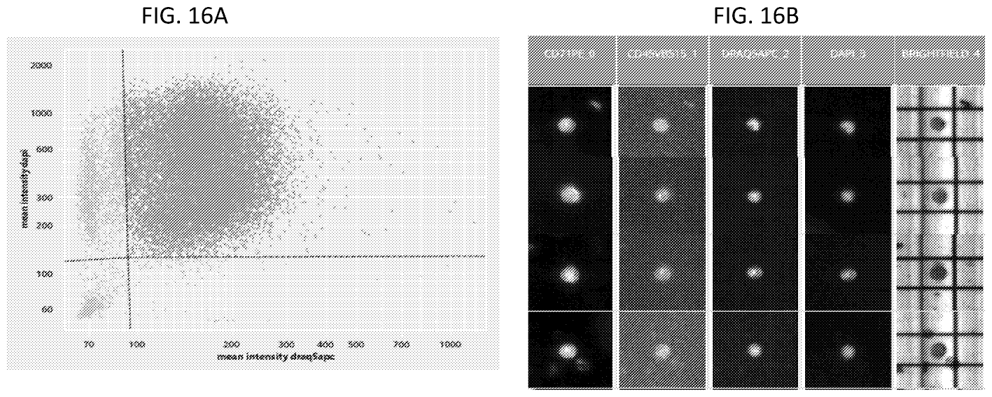

FIG. 16A shows a scatter plot analysis of Draq5/Hoechst positive erythroblasts spiked in Healthy donor blood and enriched with TREML-2-FF. FIG. 16B shows a CellBrowser® Image gallery: erythroblast cells shown positive staining from CD71-PE antibody, Draq5 and Hoechst nuclear staining, and negative staining for CD45-FITC antibody.

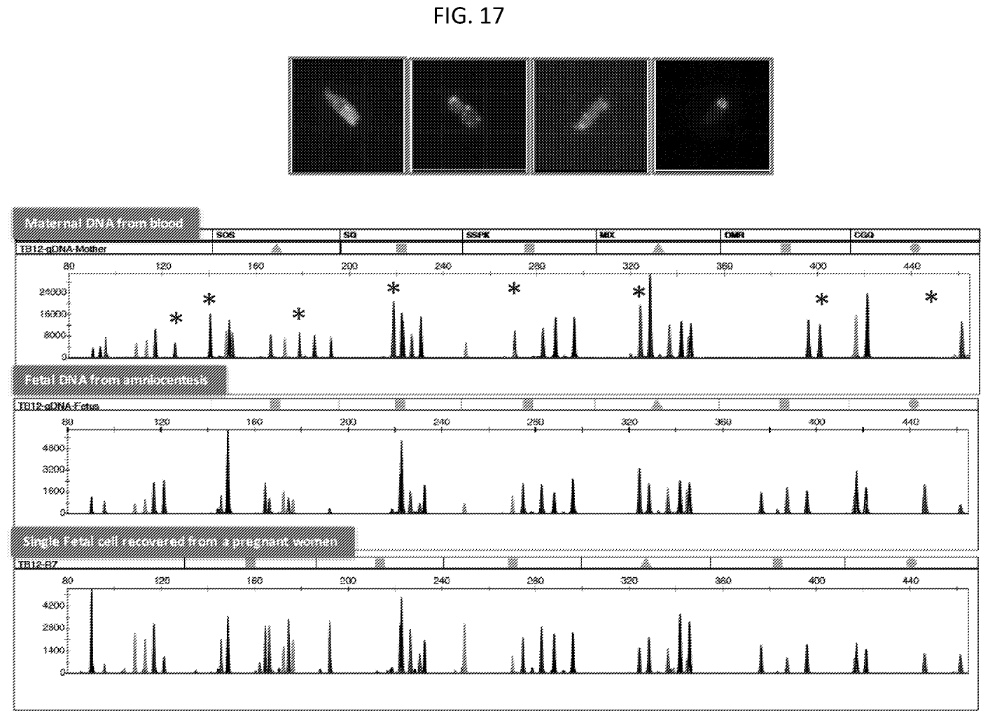

Sorted single cells were analyzed by STR (Short Tandem Repeat) technology to demonstrate their fetal cell origin (in comparison with maternal DNA as well as with fetal DNA analysis derived from the amniocentesis procedure). Identical loci profiles were detected. FIG. 17 shows a STR Analysis from single fetal cell.

In a pilot clinical study, 14 pregnant women at various gestational weeks were enrolled and fetal cells obtained from blood samples from the pregnant woman was positively detected, as shown by STR analysis.

TABLE 5
Shows a summary of the STR Analysis

| Patient | Gestestional | Fetal cells |
|---|---|---|
| ID | week | identified by STR |
| 1 | 17 + 2 | 4 |
| 2 | 12 + 2 | 1 |
| 3 | 13 + 3 | 1 |
| 4 | 14 | 1 |
| 5 | 13 + 4 | 1 |
| 6 | 13 + 3 | 1 |
| 7 | 13 + 4 | 1 |
| 8 | 12 | 1 |
| 9 | 17 | 1 |
| 10 | 13 + 4 | 1 |
| 11 | 13 + 1 | 1 |
| 12 | 12 | 3 |
| 13 | 12 | 1 |
| 14 | 12 | 2 |

To demonstrate that from single-cells recoveries of fetal cells from chorionic villus sampling we could detect chr21 trisomy sampling (VK), we performed a copy-number variant (CNV) analysis

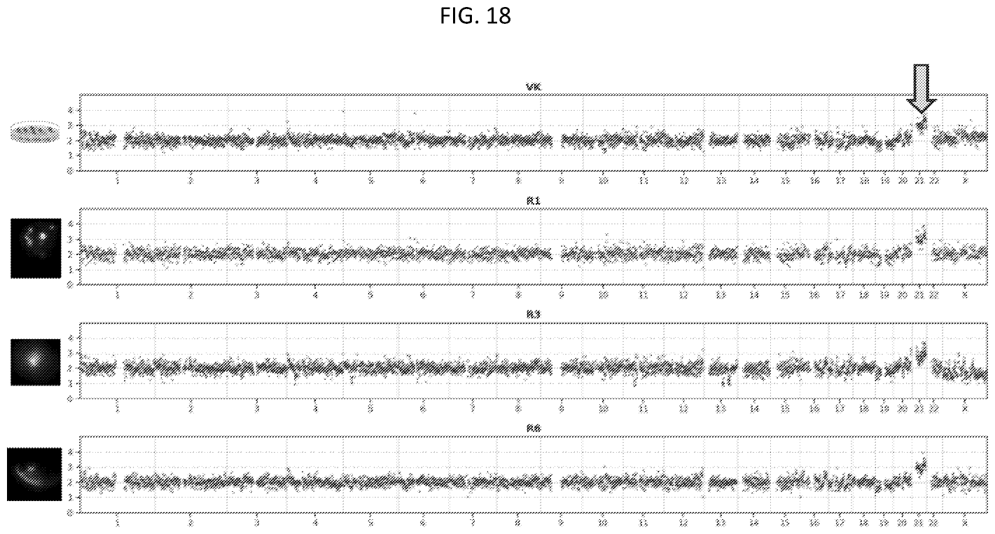

FIG. 18 shows the results of a CNV analysis of fetal cells. As shown in FIG. 18, single-cell recoveries (e.g., from recovery 1 (R1), recovery 3 (R3), and recovery 6 (R6)) from DEPArray confirm the chr21 trisomy present on the library from chorionic villus sampling (VK).

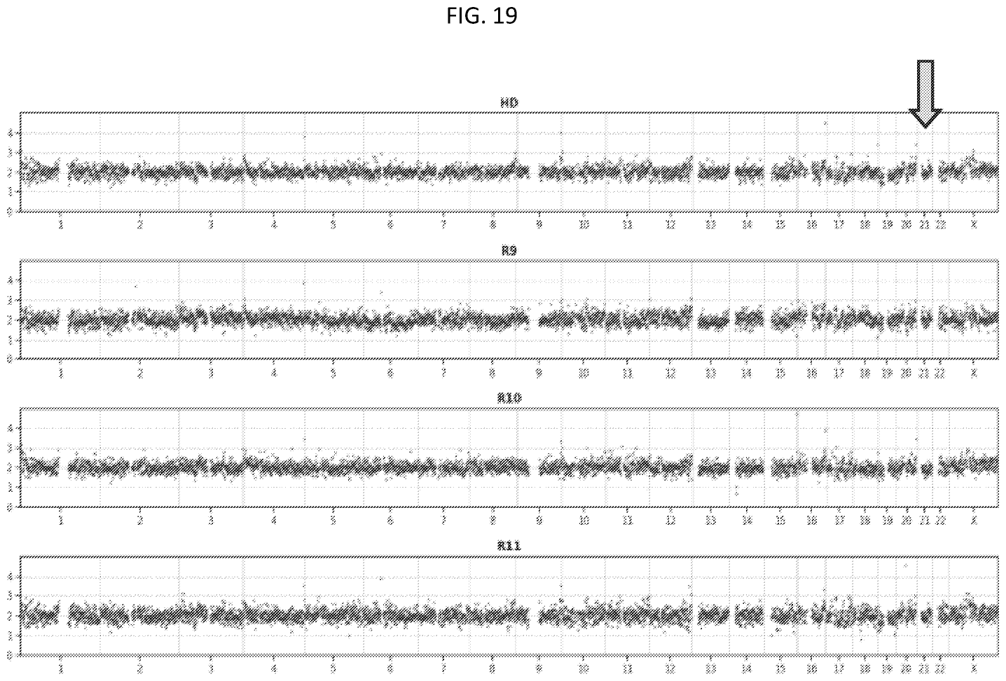

FIG. 19 shows the results of a CNV analysis for a healthy donor. As shown in FIG. 19, a healthy donor (HD) shows a flat copy-number profile, similar to those obtained by PBMC single-cells isolated from the DEPArray.

### Example 4

### Workflow Procedure for nRBC Selection From Maternal Blood

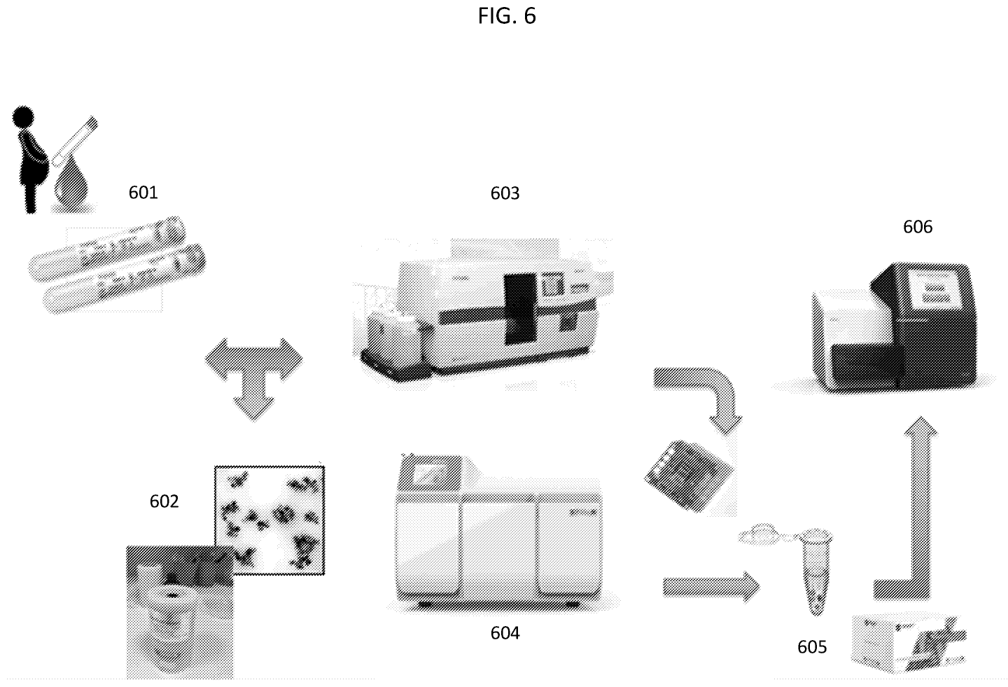

This example describes one method for selecting nucleated red blood cells (nRBC) from a blood sample from a pregnant subject. As shown in FIG. 6, a blood sample is collected from a pregnant subject (601). The blood sample is collected in CellSave tubes (601). The nRBCs can be enriched by magnetic separation (602, e.g., ferrofluid enrichment). Alternatively, or additionally, sample can be processed using a CELLTRACKS® AUTOPREP® System (603). Single cells can be visualized and isolated using a DEPArray™ NxT Control Unit Image-based technology (604). Once the cells are isolated, nucleic acids are purified from the isolated cells (605). Genomic and/or genetic analysis is performed (606). For instance, the nucleic acid molecules are sequenced to detect chromosomal abnormalities.

### Example 5

### RNA-sequencing Protocol

Nucleic acid molecules, such as RNA, may be isolated from rare cells (e.g., fetal cells). This example provides an exemplary method for sequencing RNA from fetal cells.

SMART-Seq V2

For RT-PCR, Smartseq version2 was adapted with some modifications.

For fetal erythroblasts (EB), 2 ng of total RNA input was used for Reverse Transcription reaction. For maternal EB, due to limited number of maternal EB can be sorted, all RNA was concentrated and used for Reverse Transcription reaction.

(1) Reverse Transcription

Add 1 ul oligo dT 30VN primer (10 uM) and lul of dNTP Mix (10 mM each) in the sample tubes.

Incubate the samples at 72° C. for 3 min, and immediately put on ice.

Prepare the Reverse Transcription mix on ice as follows, and add 5.7 ul into each sample.

| Components | Vol(ul)./rxn tube |
|---|---|
| Nuclease-free water | 0.29 |
| Superscript II First-Strand Buffer(5X) | 2.00 |
| DTT (0.1M) | 0.50 |
| Betaine 5M(Q Solution Qiagen) | 2.00 |
| MgCl2 (1M) | 0.06 |
| TSO (10 uM) | 0.10 |
| SuperScript II (200 U/uL) | 0.50 |
| RNase Inhibitor(40 U/uL) | 0.25 |
| Sample | 4.3 |

Incubate the reaction in a thermal cycle as follows:

| | | |
|---|---|---|
| Temperature | Time | Cycle |

| | | | |
|---|---|---|---|
| 42° C. | 90 | min | 1 cycle |
| 50° C. | 2 | min | 10 cycles |
| 42° C. | 2 | min | |
| 70° C. | 15 | min | 1 cycle |

| | |
|---|---|
| 4° C. | ∞ |

(2) PCR Pre-amplification

Prepare the PCR mix on ice as follows, and add 15 ul into each sample.

| Components | Vol. (ul) |
|---|---|
| Nuclease-free water | 2.25 |
| KAPA HiFi HotStart ReadyMix (2X) | 12.5 |
| IS PCR primers (10 uM) | 0.25 |
| Sample | 10 |

Put the samples into Thermocycler and run the following program.

| | | |
|---|---|---|
| Temperature | Time | Cycle |

| | | | |
|---|---|---|---|
| 98° C. | 3 | min | 1 cycle |
| 98° C. | 20 | sec | 15r |
| 67° C. | 15 | sec | 20 cycles |
| 72° C. | 6 | min | |
| 72° C. | 5 | min | 1 cycle |

| | |
|---|---|
| 4° C. | ∞ |

The number of PCR cycles depends on the cell type and can be increased (for cell with low RNA content) or decreased (for cells with more RNA).

(3) PCR Purification

Purify the amplified cDNA products twice using 0.5X reaction volume of AMPure XP Beads (Beckman Coulter). Quantify the purified cDNA using High Sensitivity DNA Kit on the Agilent 2100 Bioanalyzer.

(4) Illumina Nextera XT DNA Sample Preparation

Use Illumina NEXTERA XT DNA Kit to prepare the libraries, with modifications (Volume of cDNA sample, reagents and reaction volume were optimized to ¼ of the volume of manufacturer's instructions)

Dilute the cDNA accordingly to get 300 pg.

Aliquot 1.25 ul of cDNA (300 pg) into 0.2 PCR tube.

Add 2.5 ul of Tagment DNA Buffer and 1.25 ul of Amplicon Tagment Miz.

Incubate the tagmentation reaction on thermocycler at 55 degree for 5 min.

Add 1.25 ul of NT immediately and incubate at room temp for 5 min.

Add 1.25 ul of Index1, 1.25 ul of Index2, and 3.75 ul of Nextera PCR Master Mix (NPM) into tagmented DNA.

Perform amplification using the following program:

| | | |
|---|---|---|
| Temperature | Time | Cycle |

| | | | |
|---|---|---|---|
| 72° C. | 3 | min | 1 cycle |
| 95° C. | 30 | sec | 1 cycle |
| 95° C. | 10 | sec | 12 cycle |
| 55° C. | 30 | sec | |
| 72° C. | 60 | sec | |
| 72° C. | 5 | min | 1 cycle |

| | |
|---|---|
| 4° C. | ∞ |

(5) Library DNA clean up (Purification):

Add AMPure XP Beads (0.6X the reaction volume) into library DNA.

Discard the beads and keep the supernatant for the first clean up.

In the second clean up, add AMPure XP Beads (0.7X the reaction volume).

Keep the beads and elute the DNA fragments.

Successful libraries (average 400 bp) are quantified using High Sensitivity DNA Kit on Agilent 2100 Bioanalyzer.

To pooling libraries, adjust each of the library sample to 10 nM, and pool by volume.

6) Library DNA sequencing:

The libraries were submitted to sequencing facility and paired-end sequenced (2×101bp) with Illumina HiSeg™ High Output v3 system.

### Example 6

### Trophoblast Detection

This example describes a method for detecting trophoblasts. In this example, FerroFluid technology is used for cell capture and selection and DEPArray technology is used for cell sorting.

Trophoblasts are captured with FerroFluid technology and controlled aggregation. A blood sample from a pregnant subject is contacted with ferrofluid comprising colloidal magnetic particles conjugated to an anti-EpCAM antibody (EpCAM-FF) or colloidal magnetic particles conjugated to an anti-CD105 antibody (CD105-FF). The blood sample contains a plurality of cells (fetal cells and maternal cells). A first exogenous aggregation enhancing factor, such as desthiobiotin, is conjugated to the colloidal magnetic particle. A second exogenous aggregation enhancing factor, such as streptavidin, is added to the sample. Not wishing to be bound by theory, the addition of the second exogenous aggregation enhancing factor induces aggregation of the colloidal magnetic particles, thereby making it easier to isolate the fetal cells and reduce contamination with non-fetal cells. The sample is applied to a magnetic separator and EpCAM-FF or CD105-FF bound cells are isolated.

To help facilitate further analysis of the EpCAM-FF or CD105-FF bound cells, a third exogenous aggregation enhancing factor, such as biotin, is added to the isolated cells. Not wishing to be bound by theory, the addition of the third exogenous aggregation enhancing factor reverses the aggregation of the colloidal magnetic particles, which makes it easier to analyze single cells.

DEPArray technology for cell sorting: TLS1 (i.e., TREML2) is used as a candidate for staining of trophoblasts. The isolated cells are stained with fluorescently-labeled anti-TLS antibody (i.e., anti-TREML2 antibody), anti-HLA-G antibody, and cytokeratin. The isolated and stained cell sample is applied to a DEPArray catridge and analyzed using a DEPArray instrument. Cells are identified as with trophoblasts if they stain positive for TLS, HLA-G, and cytokeratin staining.

### Example 7

### Diagnosing Fetal Abnormalities

The fetal cells that are isolated or identified by any of the methods disclosed herein are further analyzed for diagnosis fetal abnormalities. Karyotype testing is performed on fetal cells to detect chromosomal abnormalities. If a chromosomal abnormality is detected, then the fetus is diagnosed with the corresponding disorder. For instance, if three copies of chromosome 21 is detected, the fetus is diagnosed with down syndrome. In another example, if three copies of chromosome 18 are detected, the fetus is diagnosed with Edwards syndrome.

| | SEQ ID | |
|---|---|---|
| Description | NO: | Sequence |
| TREML2/TLS1 (full length) | 1 | 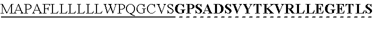 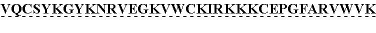 |
| | | 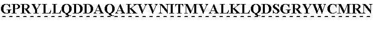 |
| | | 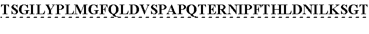 |
| | | 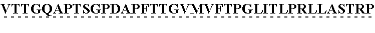 |
| | | 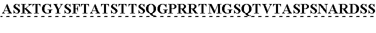 |
| | | 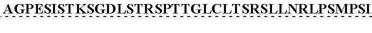 |
| | | 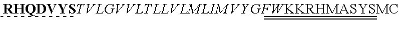 |
| | | SDPSTRDPPGRPEPYVEVYLI⁺ |
| TREML2/TLS1 (extra | 2 | GPSADSVYTKVRLLEGETLSVQCSYKGYKNRVEGKVWC |
| cellular domain) | | KIRKKKCEPGFARVWVKGPRYLLQDDAQAKVVNITMV |
| | | ALKLQDSGRYWCMRNTSGILYPLMGFQLDVSPAPQTER |
| | | NIPFTHLDNILKSGTVTTGQAPTSGPDAPFTTGVMVFTPG |
| | | LITLPRLLASTRPASKTGYSFTATSTTSQGPRRTMGSQTV |
| | | TASPSNARDSSAGPESISTKSGDLSTRSPTTGLCLTSRSLL |
| | | NRLPSMPSIRHQDVYS |
| TREML2/TLS1 | 3 | SADSVYTKVRLLEGETLSVQCSYKGYKNRVEGKVWCKI |
| fragment 1 | | RKKKCEPGFARVWVKGPRYLLQDDA |
| TREML2/TLS1 | 4 | GRYWCMRNTSGILYPLMGFQLDVSPAPQTE |
| fragment 2 | | RNIPFTEILDN ILKSGTVTTG |
| TREML2/TLS1 | 5 | TGYSFTATSTTSQGPRRTMGSQTVTASPSNARDSSAGPES |
| fragment 3 | | ISTKSGDLST |
| Anti-TREML2/TLS1 | 6 | GFSLSTSGMG |
| antibody HCVR CDR1 | | |
| Anti-TREML2/TLS1 | 7 | IWWYDDK |
| antibody HCVR CDR2 | | |
| Anti-TREML2/TLS1 | 8 | VRIESTMITGDY |
| antibody HCVR CDR3 | | |
| Anti-TREML2/TLS1 | 9 | QSVDYDGYSY |
| antibody LCVR CDR1 | | |
| Anti-TREML2/TLS1 | 10 | AAS |
| antibody LCVR CDR2 | | |
| Anti-TREML2/TLS1 | 11 | QQSIEDPWT |
| antibody LCVR CDR1 | | |

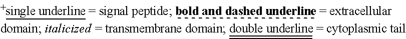

1-245. (canceled)

246. A method for isolating fetal cells from a sample from a pregnant subject, comprising:
(a) contacting the sample with a first antibody, wherein the sample comprises a plurality of cells;
(b) isolating cells bound to the first antibody to produce an enriched sample;
(c) contacting the enriched sample with a second antibody; and
(d) identifying a cell that is bound to the second antibody as a fetal cell,
wherein the first antibody or the second antibody:
(i) is an antibody that binds to a Triggering Receptor Expressed on Myeloid Cells Like 2 (TREML2) protein; or
(ii) comprises an antigen binding fragment that binds to a TREML2 protein.

247. The method of claim 246, wherein the fetal cell is a fetal nucleated red blood cell (fnRBC).

248. The method of claim 246, wherein the first antibody is conjugated to one or more magnetic particles.

249. The method of claim 248, wherein the magnetic particles are colloidal magnetic particles.

250. The method of claim 249, wherein the colloidal magnetic particles are ferrofluid magnetic particles.

251. The method of claim 248, wherein step (b) comprises subjecting the sample to a magnetic field.

252. The method of claim 251, wherein the magnetic particles are coupled to a first exogenous aggregation enhancing factor (EAEF), the first EAEF comprising one member of a specific binding pair selected from the group comprising biotin-streptavidin, antigen-antibody, receptor-hormone, receptor-ligand, agonist-antagonist, lectin-carbohydrate, Protein A-antibody Fc, and avidin-biotin, biotin analog-avidin, desthiobiotin-streptavidin,desthiobiotin-avidin, iminobiotin-streptavidin, and iminobiotin-avidin.

253. The method of claim 252, wherein step (a) comprises adding a second EAEF to induce aggregation of the magnetic particles, and wherein the second EAEF comprises the other member of the specific binding pair.

254. The method of claim 253, wherein step (b) comprises adding to the enriched sample a member of the specific binding pair in order to reverse aggregation of the magnetic particles in the enriched sample.

255. The method of claim 246, further comprising, prior to step (a), adding to the sample at least one aggregation inhibiting agent selected from the group consisting of a reducing agent, an immune-complex, a chelating agent, and a diamino butane.

256. The method of claim 255, wherein the aggregation inhibiting agent is a chelating agent, and wherein the chelating agent is EDTA 12.

257. The method of claim 246, wherein the second antibody is an antibody that binds to TREML2 protein or comprises an antigen binding fragment that binds to a TREMI,2 protein.

258. The method of claim 246, further comprising, prior to step (d), isolating single fetal cells.

259. The method of claim 258, wherein isolating single fetal cells is carried out by isolating single fetal cells that are bound to the second antibody.

260. The method of claim 259, wherein the second antibody which is conjugated to a label.

261. The method of claim 260, wherein the label is a fluorescent label.

262. The method of claim 261, wherein isolating single fetal cells is based on immunofluorescent technology.

263. The method of claim 262, wherein isolating single fetal.cells is carried out by fluorescence activated cell sorting (FACS).

264. The method of claim 262, wherein isolating single cells is carried out with a DEP Array.

265. The method of claim 246, wherein step d omprises performing a sequencing analysis.

266. The method of claim 265, wherein the sequencing analysis comprises short tandem repeat (STR) analysis.

267. The method of claim 246, further comprising performing a genomic or a genetic analysis of the fetal cell to detect the presence or absence of one or more genetic abnormalities in the fetal cell.

268. The method of claim 246, wherein the first antibody is an antibody that binds to a TREML2 protein or comprises an antigen binding fragment that binds to a TREML2 protein.

269. The method of claim 246, wherein the first antibody binds to a protein selected from EpCAM, CD105, and CD71, and the second antibody is an antibody that binds to a TREML2 protein or comprises an antigen binding fragment that binds to a TREML2 protein.

270. The method of claim 246, wherein the antibody that binds to a TREML2 protein or an antigen binding fragment that binds to a TREML2 protein comprises a heavy chain variable region (HCVR) comprising:
(i) a complementarity determining region (CDR) 1 comprising the amino acid sequence of SEQ ID NO: 6;
(ii) a HCVR CDR2 comprising the amino acid sequence of SEQ ID NO: 7;
(iii) a HCVR CDR3 comprising the amino acid sequence of SEQ ID NO: 8; and/or a light chain variable region (LCVR) comprising:
(iv) a CDR1 comprising the amino acid sequence of SEQ ID NO: 9;
(v) a CDR2 comprising the amino acid sequence of SEQ II) NO: 10; and
(vi) a CDR3 comprising the amino acid sequence of SEQ ID NO: 11.

271. The method of claim 246, wherein the antibody that binds to a TREML2 protein is selected from sc-109096, ARP49877_P050, OACA04996, AF3259, MA5-30973, PA5-47471, ABIN634968, ARIN928294, 30-552, ARIN2463297, ABIN19999041, 11655-r001, ABIN749888, bs-2737r, ABIN1999045, 11655-rp02, ABIN293207, ABIN2387613, t8282-40, ABIN4249314, nbp1-70737-20ul, and BD563661.

272. A method for isolating fetal cells in a sample from a pregnant subject, comprising:
(a) contacting the sample with a magnetic reagent, wherein the sample comprises a plurality of cells, wherein the magnetic reagent comprises a magnetic particle conjugated to a first antibody, and wherein the first antibody binds to a protein selected from EpCAM, CD105, and CD71;
(b) contacting the sample with an anti-TREML2 antibody or antigen binding fragment thereof; and
(c) identifying a cell that is bound to the anti-TREML2 antibody as a fetal cell, thereby isolating the fetal cells.

273. A kit comprising (a) an antibody that binds to a Triggering Receptor Expressed on Myeloid Cells Like 2 (TREML2) protein (anti-TREML2 antibody) or an antigen binding fragment thereof; and (b) a magnetic reagent comprising colloidal magnetic particles.

274. An anti-TREML2 antibody, or an antigen binding fragment thereof, comprising a heavy chain variable region (HCVR) comprising:
(a) a HCVR CDRI comprising the amino acid sequence of SEQ ID NO: 6;
(b) a HCVR CDR2 comprising the amino acid sequence of SEQ ID NO: 7; and
(c) a HCVR CDR3 comprising the amino acid sequence of SEQ ID NO: 8; and/or a light chain variable region comprising:
(d) a LCVR CDRI comprising the amino acid sequence of SEQ ID NO: 9;
(e) a LCVR CDR2 comprising the amino acid sequence of SEQ ID NO: 10; and
(f) a LCVR CDR3 comprising the amino acid sequence of SEQ ID NO: 11.

275. An anti-TREML2 antibody conjugate comprising (a) an anti-TREML2 antibody or antigen binding fragment thereof; and (b) a magnetic particle, wherein the magnetic particle is conjugated to the anti-TREML2 antibody.

276. A method of preparing a fetal cell sample from a maternal sample obtained from a pregnant subject, comprising:
(a) contacting the maternal sample that comprises fetal cells and maternal cells with a first antibody conjugate, wherein the first antibody conjugate comprises (i) a first antibody; and (ii) a colloidal magnetic particle, wherein the first antibody is conjugated to the colloidal magnetic particle; and
(b) isolating cells that are bound to the first antibody conjugate by subjecting the maternal sample to a magnetic field, thereby preparing a fetal cell sample.

277. A method of preparing a fetal cell sample from a maternal sample obtained from a pregnant subject, comprising contacting the maternal sample that comprises fetal cells and maternal cells with an anti-TREML2 antibody or antigen binding fragment thereof, wherein fetal cells are bound by the anti-TREML2 antibody or antigen binding fragment thereof, thereby preparing a fetal cell sample.
